(19) United States
(12) Patent Application Publication
(10) Pub. No.: US 2024/0360441 A1
(43) Pub. Date: Oct. 31, 2024

(54) METHODS AND COMPOSITIONS FOR PROTEIN EXPRESSION AND CELL DIFFERENTIATION

(71) Applicant: Uncommon Bio Limited, Cambridge (GB)

(72) Inventors: Ruth Helen Faram, Ely (GB); Rinat Nigmatullin, Cambridge (GB)

(21) Appl. No.: 18/669,954

(22) Filed: May 21, 2024

Related U.S. Application Data
(63) Continuation of application No. PCT/GB2022/052953, filed on Nov. 22, 2022.

(30) Foreign Application Priority Data
Nov. 23, 2021 (GB) 20116867.9

(51) Int. Cl. C12N 15/11; C08L 5/08; C12N 15/10

(52) U.S. Cl. CPC C12N 15/11; C08L 5/08; C12N 15/102

(57) ABSTRACT

Provided herein are methods and compositions for alteration of protein expression that can result in cell differentiation or production of proteins of biopharmaceutical relevance or engineered cells for use as therapeutic medicinal products. The compositions may comprise a saccharide, a nucleic acid molecule, and/or a polymeric material. The compositions and methods may promote or facilitate uptake of a nucleic acid by a cell. The compositions and methods may promote or facilitate differentiation of the cell.

### CROSS-REFERENCE

This application is a continuation application of International Patent Application No. PCT/GB/2022/052953, filed Nov. 22, 2022, which claims the benefit of United Kingdom Patent Application No. 2116867.9, filed Nov. 23, 2021, each of which is entirely incorporated herein by reference.

### BACKGROUND

Transfection can introduce nucleic acid molecules into eukaryotic cells. The nucleic acid molecule being transfected into the cells may promote changes in the structural and/or functional properties of the transfected cells or the progenies thereof. The cells with different structural and/or functional properties may be used in a wide variety of purposes, including but not limited to food production, diagnostics' production, biologic drug production, cell therapies, virus production, biosensors, tissue engineering, or in drug discovery. Currently available transfection methods can be limited due to a lack of effectiveness, scalability, or reliability. Currently available transfection methods may not be applicable at industrial or manufacturing scales. Currently available transfection methods can be classified as viral or non-viral methods. Non-viral transfection methods using non-viral vectors (e.g., chemical or lipid based) can result in high cytotoxicity and a high percentage of cell loss. Non-viral transfection methods can entail a high cost to practice. Non-viral vectors may suffer from low transfection efficiency and undesirable gene expression of the nucleic acids being introduced into the cells. Additionally, non-viral transfection methods may entail a narrow range of physiochemical conditions for transfection complex formation that limit the transfection efficiency, as opposed to animal-derived components. Non-viral transfection may also use carriers that are not biodegradable or biocompatible. Viral methods using viral vectors may generate immunogenic responses against the viral vectors. The viral vectors may be carcinogenic. The viral vectors may also only allow for small payloads (e.g., the size of the transfected nucleic acids) and/or entail a high cost to manufacture. Hence, current transfection methods, viral or non-viral based, are limited for their wide-spread or industrial-scale applications.

### SUMMARY

Wide-spread or industrial-scale application of cell for variety of purposes, including but not limited to food production, diagnostics' production, biologic drug production, cell therapies, virus production, biosensors, tissue engineering, or in drug discovery entails efficient transfection of nucleic acids into cells. The cells generated using the methods, compositions, or kits may have desirable expression level of the transfected nucleic acids. Provided herein are methods, compositions, and/or kits for transfection that is highly efficient and able to produce high and stable gene expression. The methods, compositions, or kits disclosed herein can be broadly applicable for a wide variety of purposes, cell types, and/or conditions without the need of using of animal derived materials or genetic modifications. The methods, compositions, or kits disclosed herein may also avoid causing immunogenic reaction and/or cancer. The methods, compositions, or kits disclosed herein may have a low cytotoxicity. The methods, compositions, or kits disclosed herein may not use animal-derived products but still provide a high transfection efficiency. The methods, compositions, or kits disclosed herein may use carriers that are biodegradable or biocompatible. The methods, compositions, or kits disclosed herein may allow for large payloads (e.g., the size of the transfected nucleic acids) and/or entail a low cost to practice/manufacture. These methods, compositions, or kits may thus overcome the limitations imposed by currently available viral or non-viral transfection and facilitate wide-spread or industrial-scale applications of transfections of cells.

Provided herein, are compositions. In an aspect, a composition comprises a saccharide and a nucleic acid molecule, which the nucleic acid molecule is configured to facilitate a change in protein expression within a cell; and a polymeric material that is configured to encapsulate or adhere to the cell. In some embodiments, the polymeric material comprises a polymer. In some embodiments, the polymer comprises a polysaccharide-based polymer, a polypeptide-based polymer, a lipid-based polymer, or a combination thereof. In some embodiments, the polysaccharide-based polymer comprises an alginate-based polymer, a gellan gum-based polymer, a cassava-based polymer, a maize-based polymer, a corn starch-based polymer, a xanthan gum-based polymer, a locust bean-based polymer, a pullulan-based polymer, a dextran-based polymer, a cellulose-based polymer, or a combination thereof. In some embodiments, the polymeric material is configured to encapsulate the cell. In some embodiments, the polymeric material comprises a hydrogel. In some embodiments, the polymeric material comprises a 2-dimensional polymer. In some embodiments, the polymer comprises a 3-dimensional polymer. In some embodiments, the polymeric material is configured to be biodegradable.

Provided herein, are compositions. In an aspect, a composition comprises a nucleic acid molecule comprising a ribonucleic acid (RNA) and a saccharide which are configured to collectively facilitate a change in protein expression within a cell. In some embodiments, the change in protein expression within the cell facilitates differentiation of the cell into a mesodermal lineage, an endodermal lineage, or an ectodermal lineage. In some embodiments, the change in protein expression within the cell facilitates adipogenic, angiogenic, cardiogenic, chondrogenic, endothelial, epithelial, hematopoietic, hepatogenic, myogenic, neurogenic, osteogenic, parenchymal, renal, or retinal differentiation of the cell. In some embodiments, the differentiation of the cell comprises transdifferentiation of the cell or directed differentiation of the cell. In some embodiments, the cell comprises a somatic cell or a naive cell. In some embodiments, the cell comprises a fat cell, a blood vessel cell, a cardiac cell, a chondrocyte, an endothelial cell, an epithelial cell, a hematopoietic cell, a hepatocyte, a muscle cell, a neuron, or an osteogenic cell. In some embodiments, the cell comprises the muscle cell. In some embodiments, the muscle cell comprises a myofiber, a myotube, a myocyte, a myoblast, a myogenic spheroid, or a muscle cell progenitor.

Provided herein, are compositions. In an aspect, a composition comprises: a saccharide and a nucleic acid molecule which are configured to collectively facilitate a change in protein expression within a cell, wherein the cell is a stem cell comprising an embryonic stem cell, an immortalized stem cell, a mesenchymal stem cell, a muscle progenitor cell, or an induced pluripotent stem cell (iPSC).

In some embodiments, the cell comprises the iPSC. In some embodiments, the nucleic acid molecule comprises a ribonucleic acid (RNA). In some embodiments, the RNA comprises a messenger ribonucleic acid (mRNA), a micro ribonucleic acid (miRNA), a transfer ribonucleic acid (tRNA), a silencing ribonucleic acid (siRNA), a self-amplifying RNA (saRNA), an endless/circular RNA (eRNA), a trans-amplifying RNA (ta-RNA), or a combination thereof. In some embodiments, the RNA comprises the mRNA, the saRNA, the eRNA, the ta-RNA, or a combination thereof. In some embodiments, the RNA encodes MYOD1, MYOG, MYF5, MYF6, PAX3, PAX7, PPARY, adiponectin, FATP1-6, FABP4, GLUT4, Leptin, AdipoR1-2, CD137, a fragment thereof, or a variant thereof. In some embodiments, the RNA is monocistronic. In some embodiments, the RNA is polycistronic. In some embodiments, the nucleic acid molecule comprises the mRNA. In some embodiments, the nucleic acid molecule comprises the saRNA In some embodiments, the nucleic acid molecule comprises the miRNA or the siRNA. In some embodiments, the miRNA or the siRNA comprises a polynucleotide sequence that facilitates a reduction of pluripotency of the cell. In some embodiments, the reduction of pluripotency of the cell facilitates the differentiation of the cell. In some embodiments, the polynucleotide sequence comprises a polynucleotide sequence that comprises POUF51 (OCT3/4), KLF4, or SOX2, or a complementary sequence thereof. In some embodiments, the nucleic acid molecule comprises a first nucleic acid molecule and a second nucleic molecule, wherein the first nucleic acid molecule is the mRNA or the saRNA, and wherein the second nucleic acid is the miRNA or the siRNA.

Provided herein, are compositions. In an aspect, a composition comprises a nucleic acid molecule and a modified saccharide, wherein the nucleic acid molecule comprises a messenger ribonucleic acid (mRNA), a self-amplifying RNA (saRNA), an endless/circular RNA (eRNA), a trans-amplifying RNA (ta-RNA), or a combination thereof; wherein the modified saccharide comprises an aliphatic aldehyde modified saccharide, an aromatic aldehyde modified saccharide, a quaternary ammonium modified saccharide, a polyamine derivatized saccharide, a saccharide modified saccharide, a saccharide with a lipid additive, or a combination thereof; and wherein the nucleic acid or the modified saccharide is configured to facilitate a change in protein expression within a cell.

In some embodiments, the modified saccharide comprises a chitosan, a hyaluronic acid, a pullulan, a heparin, an alginate, or a combination or derivative thereof. In some embodiments, the modified saccharide comprises a modified chitosan. In some embodiments, the modified saccharide comprises an aliphatic aldehyde modified saccharide, an aromatic aldehyde modified saccharide, a quaternary ammonium modified saccharide, a polyamine derivatized saccharide, a saccharide modified saccharide, saccharide a phenol modified saccharide, or a combination thereof. In some embodiments, the aliphatic aldehyde modified saccharide comprises a hexanal aldehyde modified saccharide, a heptanal aldehyde modified saccharide, an octanal aldehyde modified saccharide, a nonanal aldehyde modified saccharide, or a decanal aldehyde modified saccharide. In some embodiments, the aliphatic aldehyde modified saccharide comprises a decanal aldehyde modified saccharide. In some embodiments, the aromatic aldehyde modified saccharide comprises a benzaldehyde modified saccharide or a cinnamaldehyde modified saccharide. In some embodiments, the polyamine derivatized saccharide comprises a spermine modified saccharide, a spermidine modified saccharide, a putrescine modified saccharide, diethylethylamine modified saccharide, dimethylethylamine modified saccharide, quaternary ammonium modified saccharide, or an arginine modified saccharide. In some embodiments, the saccharide with the lipid additive comprises a saccharide with a lecithin or a saccharide with a phosphatidylcholine. In some embodiments, the saccharide modified saccharide comprises a monosaccharide modified saccharide. In some embodiments, the monosaccharide modified saccharide comprises a lactose modified saccharide, a mannose modified saccharide, a glucose modified saccharide, a galactose modified saccharide, a glucosamine modified saccharide, a sucrose modified saccharide, a xylose modified saccharide, a ribose modified saccharide, a fructose modified saccharide, or a glyceraldehyde modified saccharide. In some embodiments, the saccharide modified saccharide comprises a polysaccharide modified saccharide. In some embodiments, the polysaccharide modified saccharide comprises an anionic polysaccharide modified saccharide, a maltose modified saccharide, a reducing polysaccharide modified saccharide, or a combination thereof. In some embodiments, the anionic polysaccharide modified saccharide comprises an alginate modified saccharide, a carboxymethylated cellulose modified saccharide, a hyaluronic acid modified saccharide, a pectin modified saccharide, a pullulan modified saccharide, a starch modified saccharide, or a xanthan gum modified saccharide. In some embodiments, the reducing polysaccharide modified saccharide comprises a maltodextrin modified saccharide or a cellobiose modified saccharide. In some embodiments, the reducing polysaccharide modified saccharide comprises a cationic lipid additive. In some embodiments, the phenol modified saccharide comprises a chlorogenic acid modified saccharide, a ferulic acid modified saccharide, a caffeic acid modified saccharide, a gallic acid modified saccharide, or a combination thereof. In some embodiments, the nucleic acid molecule encodes MYOD1, MYOG, MYF5, MYF6, PAX3, PAX7, PPARY, adiponectin, FATP1-6, FABP4, GLUT4, Leptin, AdipoR1-2, CD137, a fragment thereof, or a variant thereof. In some embodiments, the nucleic acid molecule is monocistronic. In some embodiments, the nucleic acid molecule is polycistronic. In some embodiments, the composition further comprises a micro-ribonucleic acid (miRNA) or a silencing ribonucleic acid (siRNA). In some embodiments, the miRNA or the siRNA comprises a polynucleotide sequence that facilitates a reduction of pluripotency of the cell. In some embodiments, the reduction in pluripotency of the cell facilitates the differentiation of the cell. In some embodiments, the polynucleotide sequence comprises a polynucleotide sequence that comprises POUF51 (OCT3/4), KLF4, or SOX2, or a complementary sequence thereof. In some embodiments, the nucleic acid molecule comprises a first nucleic acid molecule and a second nucleic molecule, wherein the first nucleic acid molecule is the mRNA or the saRNA, and wherein the second nucleic acid is the miRNA or the siRNA.

In some embodiments, the change in the protein expression within the cell facilitates differentiation of the cell. In some embodiments, the composition further comprises a lipid, an anionic polymer, or a combination thereof. In some embodiments, the saccharide is cationic in an aqueous solution or in a neutral solution. In some embodiments, a cationic charge of cationic moieties of the saccharide comprises at least about 0.5 mequivalent of basic group per gram of the saccharide (mequiv/g). In some embodiments, the cationic charge of the cationic moieties of the saccharide comprises at most about 20 mequiv/g. In some embodiments, the saccharide comprises an average molecular mass of at least about 20 kilodaltons (kDa). In some embodiments, the saccharide comprises an average molecular mass of at most about 2000 kDa. In some embodiments, the saccharide comprises a polysaccharide. In some embodiments, the saccharide and the nucleic acid molecule are configured to self-assemble with each other. In some embodiments, the saccharide and the nucleic acid molecule are configured to form a polyplex. In some embodiments, the nucleic acid molecule is at or near a surface of the polyplex. In some embodiments, the nucleic acid molecule is encapsulated within the polyplex. In some embodiments, the polyplex is regularly shaped, irregularly shaped, or branched. In some embodiments, the polyplex is spherical or linear. In some embodiments, an apparent diameter of the polyplex is at least about 5 nanometer (nm), as measured by dynamic light scattering (DLS) or microscopy. In some embodiments, an apparent diameter of the polyplex is at most about 5000 nm, as measured by the DLS or the microscopy. In some embodiments, the polyplex further comprises a nanoparticle. In some embodiments, the nucleic acid molecule comprises an RNA-regulatory element. In some embodiments, the RNA-regulatory element comprises a transcriptional regulatory element, a co-transcriptional regulatory element, a post-transcriptional regulatory element, a translational regulatory element, or any combination thereof. In some embodiments, the RNA-regulatory element comprises a 5′-cap, 5′-untranslated region (UTR), a 3′-UTR, a poly-A tail modification, or any combination thereof. In some embodiments, the saccharide stabilizes the nucleic acid molecule. In some embodiments, the saccharide inhibits or reduces degradation of the nucleic acid molecule. In some embodiments, the saccharide inhibits or reduces nuclease degradation of the nucleic acid molecule. In some embodiments, the nucleic acid molecule comprises a chemical modification. In some embodiments, the nucleic acid molecule comprises an unlocked nucleic acid. In some embodiments, the nucleic acid molecule comprises at least two types of nucleic acids. In some embodiments, the cell comprises a mammalian cell, a bird cell, a fish cell, a mollusks cell, or an amphibian cell. In some embodiments, the mammalian cell comprises a porcine cell, a bovine cell, a bubaline cell, an ovine cell, a caprine cell, a cervine cell, a bisontine cell, a cameline cell, an elaphine cell, or a lapine cell. In some embodiments, the mammalian cell comprises the porcine cell. In some embodiments, a molar ratio of polycation amino groups of the polyplex to nucleic acid phosphate groups of the polyplex is at least about 1. In some embodiments, a molar ratio of polycation amino groups of the polyplex to nucleic acid phosphate groups of the polyplex is at most about 60. In some embodiments, the composition comprises the nucleic acid, the polymeric material, and the lipid, wherein In some embodiments, the first mass ratio is no less than 2:1; and wherein the second mass ratio is no less than 2:1. In some embodiments, the first mass ratio is no less than 100:1.

Provided herein, are methods for facilitating a change in protein expression within a cell. In an aspect, a method for facilitating a change in protein expression within a cell comprises: (a) contacting a cell with a composition comprising: i. a saccharide, and ii. a nucleic acid molecule; (b) encapsulating or adhering the cell and the composition using a polymeric material, thereby facilitating a change in protein expression within the cell.

In some embodiments, the polymeric material comprises a polymer. In some embodiments, the polymer comprises a polysaccharide-based polymer, a polypeptide-based polymer, a lipid-based polymer, or a combination thereof. In some embodiments, the polysaccharide-based polymer comprises an alginate-based polymer, a gellan gum-based polymer, a cassava-based polymer, a maize-based polymer, a corn starch-based polymer, a xanthan gum-based polymer, a locust bean-based polymer, a pullulan-based polymer, a dextran-based polymer, a cellulose-based polymer, or a combination thereof. In some embodiments, the polymeric material is configured to encapsulate the cell. In some embodiments, the polymeric material comprises a hydrogel. In some embodiments, the polymeric material comprises a 2-dimensional polymer. In some embodiments, the polymer comprises a 3-dimensional polymer. In some embodiments, the polymeric material is configured to be biodegradable.

Provided herein, are methods for facilitating a change in protein expression within a cell. In an aspect, a method for facilitating a change in protein expression within a cell comprises: contacting the cell with a composition comprising: i. a saccharide, and ii. a nucleic acid molecule comprising a ribonucleic acid (RNA); under conditions sufficient for the cell to uptake the composition, thereby facilitating the change in protein expression within the cell.

In some embodiments, the change in protein expression within the cell facilitates differentiation of the cell into a mesodermal lineage, an endodermal lineage, or an ectodermal lineage. In some embodiments, the change in protein expression within the cell facilitates adipogenic, angiogenic, cardiogenic, chondrogenic, endothelial, epithelial, hematopoietic, hepatogenic, myogenic, neurogenic, osteogenic, parenchymal, renal, or retinal differentiation of the cell. In some embodiments, the differentiation of the cell comprises transdifferentiation of the cell or directed differentiation of the cell. In some embodiments, the cell comprises a somatic cell or a naive cell. In some embodiments, the cell comprises a fat cell, a blood vessel cell, a cardiac cell, a chondrocyte, an endothelial cell, an epithelial cell, a hematopoietic cell, a hepatocyte, a muscle cell, a neuron, or an osteogenic cell. In some embodiments, the cell comprises the muscle cell. In some embodiments, the muscle cell comprises a myofiber, a myotube, a myocyte, a myoblast, a myogenic spheroid, or a muscle cell progenitor.

Provided herein, are methods for facilitating a change in protein expression within a cell. In an aspect, a method for facilitating a change in protein expression within a cell comprises: contacting the cell with a composition comprising: i. a saccharide, and ii. a nucleic acid molecule; under conditions sufficient for the stem to uptake the composition, wherein the cell comprises a stem cell comprising an embryonic stem cell, an immortalized stem cell, a mesenchymal stem cell, a muscle progenitor cell, or an induced pluripotent stem cell (iPSC), thereby facilitating the change in the protein expression in the stem cell.

In some embodiments, the cell comprises the iPSC. In some embodiments, the nucleic acid molecule comprises an ribonucleic acid (RNA). In some embodiments, the RNA comprises a messenger ribonucleic acid (mRNA), a micro ribonucleic acid (miRNA), a transfer ribonucleic acid (tRNA), a silencing ribonucleic acid (siRNA), a self-amplifying RNA (saRNA), an endless/circular RNA (eRNA), a trans-amplifying RNA (ta-RNA), or a combination thereof. In some embodiments, the RNA comprises the mRNA, the saRNA, the eRNA, the ta-RNA, or a combination thereof. In some embodiments, the RNA encodes MYOD1, MYOG, MYF5, MYF6, PAX3, PAX7, PPARY, adiponectin, FATP1-6, FABP4, GLUT4, Leptin, AdipoR1-2, CD137, a fragment thereof, or a variant thereof. In some embodiments, the RNA is monocistronic. In some embodiments, the RNA is polycistronic. In some embodiments, the nucleic acid molecule comprises the mRNA. In some embodiments, the nucleic acid molecule comprises the saRNA In some embodiments, the nucleic acid molecule comprises the miRNA or the siRNA. In some embodiments, the miRNA or the siRNA comprises a polynucleotide sequence that facilitates a reduction of pluripotency of the cell. In some embodiments, the reduction in pluripotency of the cell facilitates the differentiation of the cell. In some embodiments, the polynucleotide sequence comprises a polynucleotide sequence that comprises POUF51 (OCT3/4), KLF4, or SOX2, or a complementary sequence thereof. In some embodiments, the nucleic acid molecule comprises a first nucleic acid molecule and a second nucleic molecule, wherein the first nucleic acid molecule is the mRNA or the saRNA, and wherein the second nucleic acid is the miRNA or the siRNA.

Provided herein, are methods for facilitating a change in protein expression within a cell. In an aspect, a method for facilitating a change in protein expression within a cell comprises: contacting the cell with a composition comprising: contacting the cell with a composition comprising: i. a modified saccharide comprising an aliphatic aldehyde modified saccharide, an aromatic aldehyde modified saccharide, a quaternary ammonium modified saccharide, a polyamine derivatized saccharide, a saccharide modified saccharide, a saccharide with a lipid additive, or a combination thereof, and ii. a nucleic acid molecule comprising a messenger ribonucleic acid (mRNA), a self-amplifying RNA (saRNA), an endless/circular RNA (eRNA), a trans-amplifying RNA (ta-RNA), and a combination thereof; under conditions sufficient for the cell to uptake the composition, thereby facilitating the change in protein expression within the cell.

In some embodiments, the modified saccharide comprises a chitosan, a hyaluronic acid, a pullulan, a heparin, an alginate, or a combination or derivative thereof. In some embodiments, the modified saccharide comprises a modified chitosan. In some embodiments, the modified saccharide comprises an aliphatic aldehyde modified saccharide, an aromatic aldehyde modified saccharide, a quaternary ammonium modified saccharide, a polyamine derivatized saccharide, a saccharide modified saccharide, saccharide a phenol modified saccharide, or a combination thereof. In some embodiments, the aliphatic aldehyde modified saccharide comprises a hexanal aldehyde modified saccharide, a heptanal aldehyde modified saccharide, an octanal aldehyde modified saccharide, a nonanal aldehyde modified saccharide, or a decanal aldehyde modified saccharide. In some embodiments, the aliphatic aldehyde modified saccharide comprises a decanal aldehyde modified saccharide. In some embodiments, the aromatic aldehyde modified saccharide comprises a benzaldehyde modified saccharide or a cinnamaldehyde modified saccharide. In some embodiments, the polyamine derivatized saccharide comprises a spermine modified saccharide, a spermidine modified saccharide, a putrescine modified saccharide, diethylethylamine modified saccharide, dimethylethylamine modified saccharide, quaternary ammonium modified saccharide, or an arginine modified saccharide. In some embodiments, the saccharide with the lipid additive comprises a saccharide with a lecithin or a saccharide with a phosphatidylcholine. In some embodiments, the saccharide modified saccharide comprises a monosaccharide modified saccharide. In some embodiments, the monosaccharide modified saccharide comprises a lactose modified saccharide, a mannose modified saccharide, a glucose modified saccharide, a galactose modified saccharide, a glucosamine modified saccharide, a sucrose modified saccharide, a xylose modified saccharide, a ribose modified saccharide, a fructose modified saccharide, or a glyceraldehyde modified saccharide. In some embodiments, the saccharide modified saccharide comprises a polysaccharide modified saccharide. In some embodiments, the polysaccharide modified saccharide comprises an anionic polysaccharide modified saccharide, a maltose modified saccharide, a reducing polysaccharide modified saccharide, or a combination thereof. In some embodiments, the anionic polysaccharide modified saccharide comprises an alginate modified saccharide, a carboxymethylated cellulose modified saccharide, a hyaluronic acid modified saccharide, a pectin modified saccharide, a pullulan modified saccharide, a starch modified saccharide, or a xanthan gum modified saccharide. In some embodiments, the reducing polysaccharide modified saccharide comprises a maltodextrin modified saccharide or a cellobiose modified saccharide. In some embodiments, the reducing polysaccharide modified saccharide comprises a cationic lipid additive. In some embodiments, the phenol modified saccharide comprises a chlorogenic acid modified saccharide, a ferulic acid modified saccharide, a caffeic acid modified saccharide, a gallic acid modified saccharide, or a combination thereof. In some embodiments, the nucleic acid molecule encodes MYOD1, MYOG, MYF5, MYF6, PAX3, PAX7, PPARY, adiponectin, FATP1-6, FABP4, GLUT4, Leptin, AdipoR1-2, CD137, a fragment thereof, or a variant thereof. In some embodiments, the nucleic acid molecule is monocistronic. In some embodiments, the nucleic acid molecule is polycistronic. In some embodiments, the composition further comprises a micro-ribonucleic acid (miRNA) or a silencing ribonucleic acid (siRNA). In some embodiments, the miRNA or the siRNA comprises a polynucleotide sequence that facilitates a reduction of pluripotency of the cell. In some embodiments, the reduction in pluripotency of the cell facilitates the differentiation of the cell. In some embodiments, the polynucleotide sequence comprises a polynucleotide sequence that comprises POUF51 (OCT3/4), KLF4, or SOX2, or a complementary sequence thereof. In some embodiments, the nucleic acid molecule comprises a first nucleic acid molecule and a second nucleic molecule, wherein the first nucleic acid molecule is the mRNA or the saRNA, and wherein the second nucleic acid is the miRNA or the siRNA.

In some embodiments, the change in the protein expression within the cell facilitates differentiation of the cell. In some embodiments, the composition further comprises a lipid, an anionic polymer, or a combination thereof. In some embodiments, the saccharide is configured to be cationic in an aqueous solution or in a neutral solution. In some embodiments, a cationic charge of cationic moieties of the saccharide comprises at least about 0.5 mequivalent of basic group per gram of the saccharide (mequiv/g). In some embodiments, the cationic charge of the cationic moieties of the saccharide comprises at most about 20 mequiv/g. In some embodiments, the saccharide comprises an average molecular mass of at least about 20 kilodaltons (kDa). In some embodiments, the saccharide comprises an average molecular mass of at most about 2000 kDa. In some embodiments, the saccharide comprises a polysaccharide. In some embodiments, the saccharide and the nucleic acid molecule are configured to self-assemble with each other. In some embodiments, the saccharide and the nucleic acid molecule are configured to form a polyplex. In some embodiments, the nucleic acid molecule is at or near a surface of the polyplex. In some embodiments, the nucleic acid molecule is encapsulated within the polyplex. In some embodiments, the polyplex is regularly shaped, irregularly shaped, or branched. In some embodiments, the polyplex is spherical or linear. In some embodiments, an apparent diameter of the polyplex is at least about 5 nanometer (nm), as measured by dynamic light scattering (DLS) or microscopy. In some embodiments, an apparent diameter of the polyplex is at most about 5000 nm, as measured by the DLS or the microscopy. In some embodiments, the polyplex further comprises a nanoparticle. In some embodiments, the nucleic acid molecule comprises an RNA-regulatory element. In some embodiments, the RNA-regulatory element comprises a transcriptional regulatory element, a co-transcriptional regulatory element, a post-transcriptional regulatory element, a translational regulatory element, or any combination thereof. In some embodiments, the RNA-regulatory element comprises a 5′-cap, 5′-untranslated region (UTR), a 3′-UTR, a poly-A tail modification, or any combination thereof. In some embodiments, the saccharide is configured to stabilize the nucleic acid molecule. In some embodiments, the saccharide is configured to inhibit or reduce degradation of the nucleic acid molecule. In some embodiments, the saccharide is configured to inhibit a or reduce nuclease degradation of the nucleic acid molecule. In some embodiments, the nucleic acid molecule comprises a chemical modification. In some embodiments, the nucleic acid molecule comprises an unlocked nucleic acid. In some embodiments, the nucleic acid molecule comprises at least two types of nucleic acids. In some embodiments, the cell comprises a mammalian cell, a bird cell, a fish cell, a mollusks cell, or an amphibian cell. In some embodiments, the mammalian cell comprises a porcine cell, a bovine cell, a bubaline cell, an ovine cell, a caprine cell, a cervine cell, a bisontine cell, a cameline cell, an elaphine cell, or a lapine cell. In some embodiments, the mammalian cell comprises the porcine cell. In some embodiments, a molar ratio of polycation amino groups of the polyplex to nucleic acid phosphate groups of the polyplex is at least about 1. In some embodiments, a molar ratio of polycation amino groups of the polyplex to nucleic acid phosphate groups of the polyplex is at most about 60. In some embodiments, the composition comprises the nucleic acid, the polymeric material, and the lipid, wherein In some embodiments, the first mass ratio is no less than 2:1; and wherein the second mass ratio is no less than 2:1. In some embodiments, the first mass ratio is no less than 100:1. In some embodiments, a ratio of the nucleic acid molecule and the cell in the contacting is at least about 0.001 ng per 10000 cells. In some embodiments, a ratio of the nucleic acid molecule and the cell in the contacting is at least about 0.01 ng per 10000 cells. In some embodiments, a ratio of the nucleic acid molecule and the cell in the contacting is at least about 0.1 ng per 10000 cells. In some embodiments, the change of protein expression within the cell comprises an increased expression of a transcript encoded by the nucleic acid molecule within the cell. In some embodiments, the transcript encoded by the nucleic acid molecule within the cell is at least about 1%, 5%, 10%, 50%, 100%, 150%, 2-fold, 5-fold, 10-fold, 100-fold, or 1000-fold higher than (1) a level of the transcript within a cell that has not contacted with the saccharide; (2) a level of the transcript within a cell that has not contacted with the nucleic acid molecule; or (3) a combination thereof, as measured by quantitative real-time polymerase chain reaction (QRT-PCR). In some embodiments, the change of protein expression within the cell comprises an increased expression of a transcript not encoded by the nucleic acid molecule within the cell. In some embodiments, the transcript not encoded by the nucleic acid molecule within the cell is at least about 1%, 5%, 10%, 50%, 100%, 150%, 2-fold, 5-fold, 10-fold, 100-fold, or 1000-fold higher than (1) a level of the transcript within a cell that has not contacted with the saccharide; (2) a level of the transcript within a cell that has not contacted with the nucleic acid molecule; or (3) a combination thereof, as measured by quantitative real-time polymerase chain reaction (QRT-PCR). In some embodiments, the change of protein expression within the cell comprises a decreased expression of a transcript not encoded by the nucleic acid molecule within the cell. In some embodiments, the transcript not encoded by the nucleic acid molecule within the cell is at least about 1%, 5%, 10%, 20%, 30%, 40% 50%, 60%, 70%, 80%, 90%, or 99% lower than (1) a level of the transcript within a cell that has not contacted with the saccharide; (2) a level of the transcript within a cell that has not contacted with the nucleic acid molecule; or (3) a combination thereof, as measured by quantitative real-time polymerase chain reaction (QRT-PCR).

Provided herein, are editable products prepared by any of the methods disclosed herein for facilitating a change in protein expression within a cell.

Provided herein, are pharmaceutically active agents prepared by any of the methods disclosed herein for facilitating a change in protein expression within a cell.

Provided herein, are tissues prepared by any of the methods disclosed herein for facilitating a change in protein expression within a cell.

Provided herein, are compositions. In an aspect, a composition comprises a saccharide and a nucleic acid molecule, which the saccharide or the nucleic acid molecule is configured to facilitate a change in protein expression in a cell, and a polymeric material that is configured to encapsulate, self-assemble, or adhere to the cell.

Provided herein, are compositions. In an aspect, a composition comprises a saccharide and a nucleic acid molecule, which the saccharide or the nucleic acid molecule is configured to promote differentiation of a cell, and a polymeric material that is configured to encapsulate, self-assemble, or adhere to the cell.

In some embodiments, the polymeric material comprises a polymer. In some embodiments, the polymer comprises a polysaccharide-based polymer, a polypeptide-based polymer, a lipid-based polymer, or a combination thereof. In some embodiments, the polysaccharide-based polymer comprises an alginate-based polymer, a gellan gum-based polymer, a cassava-based polymer, a maize-based polymer, a corn starch-based polymer, a xanthan gum-based polymer, a locust bean-based polymer, a pullulan-based polymer, a dextran-based polymer, a cellulose-based polymer, or a combination thereof. In some embodiments, the polymeric material is configured to encapsulate the cell. In some embodiments, the polymer comprises a hydrogel. In some embodiments, the polymer comprises a 2-dimensional polymer. In some embodiments, the polymer comprises a 3-dimensional polymer. In some embodiments, the polymer is configured to be biodegradable.

In some embodiments, the change in the protein expression in the cell facilitates differentiation of the cell. In some embodiments, the composition further comprises a lipid, an anionic polymer, or a combination thereof. In some embodiments, the saccharide is configured to be cationic in an aqueous solution. In some embodiments, a cationic charge of cationic moieties of the saccharide comprises at least about 0.5 mequivalent of basic group per gram of polymer (mequiv/g). In some embodiments, a cationic charge of cationic moieties of the saccharide comprises at most about 20 mequiv/g. In some embodiments, the saccharide comprises an average molecular mass of at least about 20 kilodaltons (kDa). In some embodiments, the saccharide comprises an average molecular mass of at most about 2000 kDa. In some embodiments, the saccharide comprises a polysaccharide.

In some embodiments, the saccharide and the nucleic acid molecule are configured to self-assemble with each other. In some embodiments, the saccharide and the nucleic acid molecule are configured to form a polyplex. In some embodiments, the nucleic acid molecule is configured to be adsorbed to a surface of the polyplex. In some embodiments, the nucleic acid molecule is configured to be encapsulated within the polyplex. In some embodiments, the polyplex is regularly shaped, irregularly shaped, or branched. In some embodiments, the polyplex is spherical or linear. In some embodiments, an apparent diameter of the polyplex is at least about 5 nanometer (nm), as measured by dynamic light scattering (DLS) or microscopy. In some embodiments, an apparent diameter of the polyplex is at most about 5000 nm, as measured by DLS or microscopy. In some embodiments, the polyplex comprises a nanoparticle. In some embodiments, the saccharide is configured to stabilize the nucleic acid molecule. In some embodiments, the saccharide is configured to inhibit or reduce degradation of the nucleic acid molecule. In some embodiments, the polymeric material that is configured to encapsulate the cell.

In some embodiments, the nucleic acid molecule comprises an RNA. In some embodiments, the RNA comprises messenger ribonucleic acid (mRNA), micro ribonucleic acid (miRNA), transfer ribonucleic acid (tRNA), silencing ribonucleic acid (siRNA), self-amplifying RNA (saRNA), endless/circular RNA (eRNA), trans-amplifying RNA (ta-RNA), or a combination thereof. In some embodiments, the RNA is monocistronic or polycistronic. In some embodiments, the RNA comprises an RNA-regulatory element. In some embodiments, the RNA-regulatory element comprises a transcriptional regulatory element, a co-transcriptional regulatory element, a post-transcriptional regulatory element, a translational regulatory element, or any combination thereof. In some embodiments, the RNA-regulatory element comprises a 5′-cap, a 5′-untranslated region (UTR), a 3′-UTR, a poly-A tail modification, or any combination thereof. In some embodiments, the nucleic acid molecule comprises the mRNA, the miRNA or the siRNA. In some embodiments, the miRNA or the siRNA comprises a polynucleotide sequence that facilitates a reduction of pluripotency of the cell. In some embodiments, the nucleic acid molecule is chemically modified. In some embodiments, the nucleic acid molecule comprises an unlocked nucleic acid. In some embodiments, the nucleic acid molecule comprises at least two types of nucleic acids. In some embodiments, a molar ratio of polycation amino groups of the polyplex to nucleic acid phosphate groups of the polyplex is at least about 1. In some embodiments, a molar ratio of polycation amino groups of the polyplex to nucleic acid phosphate groups of the polyplex is at most about 60.

Provided herein, are compositions. In an aspect, a composition comprises a nucleic acid molecule comprising an RNA and a saccharide which are configured to collectively facilitate a change in protein expression in a cell.

Provided herein, are compositions. In an aspect, a composition comprises a nucleic acid molecule comprising an mRNA and a saccharide which are configured to promote mesodermal, endodermal and ectodermal lineages as follows: adipogenic, angiogenic, cardiogenic, chondrogenic, endothelial, epithelial, hematopoietic, hepatogenic, myogenic, neurogenic, osteogenic, parenchymal, renal, or retinal differentiation of a cell.

In some embodiments, the change in the protein expression in the cell facilitates mesodermal, endodermal and ectodermal lineages as follows: adipogenic, angiogenic, cardiogenic, chondrogenic, endothelial, epithelial, hematopoietic, hepatogenic, myogenic, neurogenic, osteogenic, parenchymal, renal, or retinal differentiation of the cell. In some embodiments, the differentiation comprises transdifferentiation of the cell or directed differentiation of the cell. In some embodiments, the cell comprises a somatic cell or a naive cell. In some embodiments, the cell comprises a fat cell, a blood vessel cell, a cardiac cell, a chondrocyte, an endothelial cell, an epithelial cell, a hematopoietic cell, a hepatocyte, a muscle cell, a neuron, or an osteogenic cell. In some embodiments, the muscle cell comprises a myofiber, a myotube, a myocyte, a myoblast, a myogenic spheroid, or a muscle cell progenitor. In some embodiments, the cell comprises an animal cell. In some embodiments, the animal cell comprises a mammalian cell, a bird cell, a fish cell, a mollusks cell, or an amphibian cell. In some embodiments, the mammalian cell comprises a porcine cell, a bovine cell, a bubaline cell, an ovine cell, a caprine cell, a cervine cell, a bisontine cell, a cameline cell, an elaphine cell, or a lapine cell. In some embodiments, the bird cell comprises an anatine cell, a galline cell, an anserine cell, a meleagrine cell, a struthionine cell, or a phasianine cell.

In some embodiments, the mRNA encodes MYOD1, MYOG, MYF5, MYF6, PAX3, PAX7, PPARY, adiponectin, FATP1-6, FABP4, GLUT4, Leptin, AdipoR1-2, CD137, a fragment thereof, or a variant thereof. In some embodiments, the composition further comprises a lipid, an anionic polymer, or a combination thereof. In some embodiments, the RNA is monocistronic or polycistronic. In some embodiments, the RNA comprises an RNA-regulatory element. In some embodiments, the RNA-regulatory element comprises a transcriptional regulatory element, a co-transcriptional regulatory element, a post-transcriptional regulatory element, a translational regulatory element, or any combination thereof. In some embodiments, the RNA-regulatory element comprises a 5′-cap, a 5′-untranslated region (UTR), a 3′-UTR, a poly-A tail modification, or any combination thereof. In some embodiments, the RNA comprises messenger RNA (mRNA), micro ribonucleic acid (miRNA), transfer ribonucleic acid (tRNA), silencing ribonucleic acid (siRNA), self-amplifying RNA (saRNA), endless/circular RNA (eRNA), trans-amplifying RNA (ta-RNA), or a combination thereof. In some embodiments, the RNA comprises the mRNA. In some embodiments, the miRNA or the siRNA comprises a polynucleotide sequence that facilitates a reduction of pluripotency of the cell. In some embodiments, the reduction in pluripotency of the cell facilitates the differentiation of the cell. In some embodiments, the polynucleotide sequence comprises a polynucleotide sequence that comprises POUF51 (OCT3/4), KLF4, or SOX2, or a complementary sequence thereof.

In some embodiments, the saccharide is configured to be cationic in an aqueous solution. In some embodiments, the wherein the saccharide is configured to be cationic in a neutral aqueous solution. In some embodiments, a cationic charge of cationic moieties of the saccharide comprises at least about 0.5 mequivalent of basic group per gram of polymer (mequiv/g). In some embodiments, a cationic charge of cationic moieties of the saccharide comprises at most about 20 mequiv/g. In some embodiments, the saccharide comprises an average molecular mass of at least about 20 kilodaltons (kDa). In some embodiments, the saccharide comprises an average molecular mass of at most about 2000 kDa. In some embodiments, the saccharide comprises a polysaccharide.

In some embodiments, the saccharide and the RNA are configured to self-assemble with each other. In some embodiments, the saccharide and the RNA are configured to form a polyplex. In some embodiments, the RNA is configured to be adsorbed to a surface of the polyplex. In some embodiments, the RNA is configured to be encapsulated within the polyplex. In some embodiments, the polyplex is regularly shaped, irregularly shaped, or branched. In some embodiments, the polyplex is spherical or linear. In some embodiments, an apparent diameter of the polyplex is at least about 5 nanometer (nm), as measured by dynamic light scattering (DLS) or microscopy. In some embodiments, an apparent diameter of the polyplex is at most about 5000 nm, as measured by DLS or microscopy. In some embodiments, the polyplex comprises a nanoparticle.

In some embodiments, the saccharide is configured to stabilize the RNA. In some embodiments, the saccharide is configured to inhibit or reduce degradation of the RNA. In some embodiments, the saccharide is configured to inhibit a or reduce nuclease degradation of the RNA. In some embodiments, the RNA is chemically modified. In some embodiments, the RNA comprises an unlocked nucleic acid. In some embodiments, the RNA comprises at least two types of nucleic acids. In some embodiments, a molar ratio of the saccharide to the RNA is at least about 1. In some embodiments, a molar ratio of the saccharide to the RNA is at most about 60.

Provided herein, are compositions. In an aspect, a composition comprises a saccharide and a nucleic acid molecule which are configured to collectively facilitate a change in protein expression in a stem cell, wherein the stem cell comprises an embryonic stem cell, an immortalized stem cell, a mesenchymal stem cell, a muscle progenitor cell, or an induced pluripotent stem cell (iPSC), thereby facilitating the change in the protein expression in the stem cell.

Provided herein, are compositions. In an aspect, a composition comprises a saccharide and a nucleic acid molecule which are configured to collectively promote differentiation an induced pluripotent stem cell (iPSC), thereby facilitating the change in the protein expression in the iPSC.

In some embodiments, the stem cell comprises the iPSC. In some embodiments, the change in the protein expression in the stem cell facilitates differentiation of the stem cell. In some embodiments, the animal stem cell comprises a mammalian stem cell, a bird stem cell, a fish stem cell, a mollusk stem cell, or an amphibian stem cell. In some embodiments, the mammalian stem cell comprises a porcine stem cell, a bovine stem cell, a bubaline stem cell, an ovine stem cell, a caprine stem cell, a cervine stem cell, a bisontine stem cell, a cameline stem cell, an elaphine stem cell, or a lapine stem cell. In some embodiments, the mammalian stem cell comprises the porcine stem cell. In some embodiments, the bird stem cell comprises an anatine stem cell, a galline stem cell, an anserine stem cell, a meleagrine stem cell, a struthionine stem cell, or a phasianine stem cell. In some embodiments, the composition further comprises a lipid, an anionic polymer, or a combination thereof.

In some embodiments, the saccharide is configured to be cationic in an aqueous solution. In some embodiments, a cationic charge of cationic moieties of the saccharide comprises at least about 0.5 mequivalent of basic group per gram of polymer (mequiv/g). In some embodiments, a cationic charge of cationic moieties of the saccharide comprises at most about 20 mequiv/g. In some embodiments, the saccharide comprises an average molecular mass of at least about 20 kilodaltons (kDa). In some embodiments, the saccharide comprises an average molecular mass of at most about 2000 kDa. In some embodiments, the saccharide comprises a polysaccharide.

In some embodiments, the saccharide and the nucleic acid molecule are configured to self-assemble with each other. In some embodiments, the saccharide and the nucleic acid molecule are configured to form a polyplex. In some embodiments, the nucleic acid molecule is configured to be adsorbed to a surface of the polyplex. In some embodiments, the nucleic acid molecule is configured to be encapsulated within the polyplex. In some embodiments, the polyplex is regularly shaped, irregularly shaped, or branched. In some embodiments, the polyplex is spherical or linear. In some embodiments, an apparent diameter of the polyplex is at least about 5 nanometer (nm), as measured by dynamic light scattering (DLS) or microscopy. In some embodiments, an apparent diameter of the polyplex is at most about 5000 nm, as measured by DLS or microscopy. In some embodiments, the polyplex comprises a nanoparticle. In some embodiments, the saccharide is configured to stabilize the nucleic acid molecule. In some embodiments, the saccharide is configured to inhibit or reduce degradation of the nucleic acid molecule. In some embodiments, the saccharide is configured to inhibit a or reduce nuclease degradation of the nucleic acid molecule.

In some embodiments, the nucleic acid molecule an RNA. In some embodiments, the RNA comprises, micro ribonucleic acid (miRNA), transfer ribonucleic acid (tRNA), silencing ribonucleic acid (siRNA), self-amplifying RNA (saRNA), endless/circular RNA (eRNA), trans-amplifying RNA (ta-RNA), or a combination thereof. In some embodiments, the RNA is monocistronic or polycistronic. In some embodiments, the RNA comprises an RNA-regulatory element. In some embodiments, the RNA-regulatory element comprises a transcriptional regulatory element, a co-transcriptional regulatory element, a post-transcriptional regulatory element, a translational regulatory element, or any combination thereof. In some embodiments, the RNA-regulatory element comprises a 5′-cap, 5′-untranslated region (UTR), a 3′-UTR, a poly-A tail modification, or any combination thereof. In some embodiments, the nucleic acid molecule comprises the miRNA or the siRNA. In some embodiments, the miRNA or the siRNA comprises a polynucleotide sequence that facilitates a reduction of pluripotency of the stem cell.

In some embodiments, the nucleic acid molecule is chemically modified. In some embodiments, the nucleic acid molecule comprises an unlocked nucleic acid. In some embodiments, the nucleic acid molecule comprises at least two types of nucleic acids. In some embodiments, a molar ratio of polycation amino groups of the polyplex to nucleic acid phosphate groups of the polyplex is at least about 1 or about at most 60. In some embodiments, the nucleic acid molecule comprises messenger ribonucleic acid (mRNA).

Provided herein, are compositions. In an aspect, a composition comprises a nucleic acid molecule comprising a messenger ribonucleic acid (mRNA), and a modified saccharide comprising an aliphatic aldehyde modified saccharide, an aromatic aldehyde modified saccharide, a quaternary ammonium modified saccharide, a polyamine derivatized saccharide, a saccharide modified saccharide, a saccharide with a lipid additive, or a combination thereof; wherein the nucleic acid or the modified saccharide is configured to facilitate a change in protein expression in a cell.

Provided herein, are compositions. In an aspect, a composition comprises a nucleic acid molecule comprising a messenger ribonucleic acid (mRNA), and a modified saccharide comprising an aliphatic aldehyde modified saccharide, an aromatic aldehyde modified saccharide, a quaternary ammonium modified saccharide, a polyamine derivatized saccharide, a saccharide modified saccharide, a saccharide with a lipid additive, or a combination thereof; wherein the nucleic acid or the modified saccharide is configured to promote differentiation of a cell.

In some embodiments, the modified saccharide comprises a chitosan, a hyaluronic acid, a pullulan, a heparin, an alginate, or a combination or derivative thereof. In some embodiments, wherein the modified saccharide comprises a modified chitosan. In some embodiments, the modified saccharide comprises an aliphatic aldehyde modified saccharide, an aromatic aldehyde modified saccharide, a quaternary ammonium modified saccharide, a polyamine derivatized saccharide, a saccharide modified saccharide, saccharide a phenol modified saccharide, or a combination thereof. In some embodiments, the aliphatic aldehyde modified saccharide comprises a hexanal aldehyde modified saccharide, a heptanal aldehyde modified saccharide, an octanal aldehyde modified saccharide, a nonanal aldehyde modified saccharide, or a decanal aldehyde modified saccharide. In some embodiments, the aliphatic aldehyde modified saccharide comprises a decanal aldehyde modified saccharide. In some embodiments, the aromatic aldehyde modified saccharide comprises a benzaldehyde modified saccharide or a cinnamaldehyde modified saccharide. In some embodiments, the polyamine derivatized saccharide comprises a spermine modified saccharide, a spermidine modified saccharide, a putrescine modified saccharide, diethylethylamine modified saccharide, dimethylethylamine modified saccharide, quaternary ammonium modified saccharide, or an arginine modified saccharide. In some embodiments, the saccharide with the lipid additive comprises a saccharide with a lecithin or a saccharide with a phosphatidylcholine. In some embodiments, the saccharide modified saccharide comprises a monosaccharide modified saccharide or a polysaccharide modified saccharide. In some embodiments, the monosaccharide modified saccharide comprises a lactose modified saccharide, a mannose modified saccharide, a glucose modified saccharide, a galactose modified saccharide, a glucosamine modified saccharide, a sucrose modified saccharide, a maltose modified saccharide, a xylose modified saccharide, a ribose modified saccharide, a fructose modified saccharide, or a glyceraldehyde modified saccharide. In some embodiments, the polysaccharide modified saccharide comprises an anionic polysaccharide modified saccharide or a reducing polysaccharide modified saccharide. In some embodiments, the anionic polysaccharide modified saccharide comprises an alginate modified saccharide, a carboxymethylated cellulose modified saccharide, a hyaluronic acid modified saccharide, a pectin modified saccharide, a pullulan modified saccharide, a starch modified saccharide, or a xanthan gum modified saccharide. In some embodiments, the reducing polysaccharide modified saccharide comprises a maltodextrin modified saccharide or a cellobiose modified saccharide. In some embodiments, the reducing polysaccharide modified saccharide comprises a cationic lipid additive. In some embodiments, the phenol modified saccharide comprises a chlorogenic acid modified saccharide, a ferulic acid modified saccharide, a caffeic acid modified saccharide, a gallic acid modified saccharide, or a combination thereof.

In some embodiments, the change in the protein expression in the cell facilitates differentiation of the cell. In some embodiments, the saccharide is configured to be cationic in an aqueous solution. In some embodiments, the saccharide is configured to be cationic in a neutral aqueous solution. In some embodiments, a cationic charge of cationic moieties of the saccharide comprises at least about 0.5 mequivalent of basic group per gram of polymer (mequiv/g). In some embodiments, a cationic charge of cationic moieties of the saccharide comprises at most about 20 mequiv/g. In some embodiments, the saccharide comprises an average molecular mass of at least about 20 kilodaltons (kDa). In some embodiments, the saccharide comprises an average molecular mass of at most about 2000 kDa. In some embodiments, the saccharide comprises a polysaccharide.

In some embodiments, the saccharide and the mRNA are configured to self-assemble with each other. In some embodiments, the saccharide and the mRNA are configured to form a polyplex. In some embodiments, the mRNA is configured to be adsorbed to a surface of the polyplex. In some embodiments, the mRNA is configured to be encapsulated within the polyplex. In some embodiments, the polyplex is regularly shaped, irregularly shaped, or branched. In some embodiments, the polyplex is spherical or linear. In some embodiments, an apparent diameter of the polyplex is at least about 5 nanometer (nm), as measured by dynamic light scattering (DLS) or microscopy. In some embodiments, an apparent diameter of the polyplex is at most about 5000 nm, as measured by DLS or microscopy. In some embodiments, the polyplex comprises a nanoparticle. In some embodiments, the saccharide is configured to stabilize the mRNA. In some embodiments, the saccharide is configured to inhibit or reduce degradation of the mRNA.

In some embodiments, the saccharide is configured to inhibit a or reduce nuclease degradation of the mRNA. In some embodiments, the mRNA is monocistronic or polycistronic. In some embodiments, the mRNA comprises a non-coding sequence. In some embodiments, the mRNA comprises an RNA-regulatory element. In some embodiments, the RNA-regulatory element comprises a transcriptional regulatory element, a co-transcriptional regulatory element, a post-transcriptional regulatory element, a translational regulatory element, or any combination thereof. In some embodiments, the RNA-regulatory element comprises a 5′-cap, 5′-untranslated region (UTR), a 3′-UTR, a poly-A tail modification, or any combination thereof. In some embodiments, composition further comprises micro ribonucleic acid (miRNA), transfer ribonucleic acid (tRNA), silencing ribonucleic acid (siRNA), self-amplifying RNA (saRNA), endless/circular RNA (eRNA), trans-amplifying RNA (ta-RNA), or a combination thereof. In some embodiments, composition further comprises the miRNA or the siRNA. In some embodiments, the miRNA or the siRNA comprises a polynucleotide sequence that facilitates a reduction of pluripotency of the cell.

In some embodiments, the mRNA is chemically modified. In some embodiments, the mRNA comprises an unlocked nucleic acid. In some embodiments, the mRNA comprises at least two types of nucleic acids. In some embodiments, a molar ratio of the saccharide to the mRNA is at least about 1. In some embodiments, a molar ratio of the saccharide to the mRNA is at most about 60.

Provided herein, are methods for facilitating a change in protein expression in a cell. In an aspect, a method for facilitating a change in protein expression in a cell comprises (a) contacting a cell with a composition comprising: (i) a saccharide, and (ii) a nucleic acid molecule; (b) encapsulating, self-assembling, or adhering to the cell and the composition using a polymeric material, thereby facilitating the change in the protein expression in the cell.

Provided herein, are methods for differentiating a cell. In an aspect, a method for differentiating a cell comprises (a) contacting a cell with a composition comprising: (i) a saccharide, and (ii) a nucleic acid molecule; (b) encapsulating, self-assembling, or adhering to the cell and the composition using a polymeric material, thereby facilitating differentiation of the cell.

In some embodiments, the polymeric material comprises a polymer. In some embodiments, the polymer comprises a polysaccharide-based polymer, a polypeptide-based polymer, a lipid-based polymer, or a combination thereof. In some embodiments, the polysaccharide-based polymer comprises an alginate-based polymer, a gellan gum-based polymer, a cassava-based polymer, a maize-based polymer, a corn starch-based polymer, a xanthan gum-based polymer, a locust bean-based polymer, a pullulan-based polymer, a dextran-based polymer, a cellulose-based polymer, or a combination thereof. In some embodiments, (b) comprises encapsulating the cell using the polymeric material. In some embodiments, the polymer comprises a hydrogel. In some embodiments, the polymer comprises a 2-dimensional polymer. In some embodiments, the polymer comprises a 3-dimensional polymer. In some embodiments, the polymer is configured to be biodegradable. In some embodiments, the change in protein expression in the cell facilitates differentiation of the cell. In some embodiments, the composition further comprises a lipid, an anionic polymer, or a combination thereof.

In some embodiments, the saccharide is configured to be cationic in an aqueous solution. In some embodiments, a cationic charge of cationic moieties of the saccharide comprises at least about 0.5 mequivalent of basic group per gram of polymer (mequiv/g). In some embodiments, a cationic charge of cationic moieties of the saccharide comprises at most about 20 mequiv/g. In some embodiments, the saccharide comprises an average molecular mass of at least about 20 kilodaltons (kDa). In some embodiments, the saccharide comprises an average molecular mass of at most about 2000 kDa. In some embodiments, the saccharide comprises a polysaccharide. In some embodiments, the saccharide and the nucleic acid molecule are configured to self-assemble with each other. In some embodiments, the saccharide and the nucleic acid molecule are configured to form a polyplex. In some embodiments, the nucleic acid molecule is configured to be adsorbed to a surface of the polyplex. In some embodiments, the nucleic acid molecule is configured to be encapsulated within the polyplex. In some embodiments, the polyplex is regularly shaped, irregularly shaped, or branched. In some embodiments, the polyplex is spherical or linear. In some embodiments, an apparent diameter of the polyplex is at least about 5 nanometer (nm), as measured by dynamic light scattering (DLS) or microscopy. In some embodiments, an apparent diameter of the polyplex is at most about 5000 nm, as measured by DLS or microscopy. In some embodiments, the polyplex comprises a nanoparticle. In some embodiments, the saccharide is configured to stabilize the nucleic acid molecule. In some embodiments, the saccharide is configured to inhibit or reduce degradation of the nucleic acid molecule. In some embodiments, (b) comprises encapsulating the cell and the composition using the polymeric material.

In some embodiments, the nucleic acid molecule comprises an RNA. In some embodiments, the RNA comprises messenger ribonucleic acid (mRNA), micro ribonucleic acid (miRNA), transfer ribonucleic acid (tRNA), silencing ribonucleic acid (siRNA), self-amplifying RNA (saRNA), endless/circular RNA (eRNA), trans-amplifying RNA (ta-RNA), or a combination thereof. In some embodiments, the RNA is monocistronic or polycistronic. In some embodiments, the RNA comprises an RNA-regulatory element. In some embodiments, the RNA-regulatory element comprises a transcriptional regulatory element, a co-transcriptional regulatory element, a post-transcriptional regulatory element, a translational regulatory element, or any combination thereof. In some embodiments, the RNA-regulatory element comprises a 5′-cap, 5′ untranslated region (UTR), a 3′-UTR, a poly-A tail modification, or any combination thereof. In some embodiments, the nucleic acid molecule comprises the mRNA, the miRNA, or the siRNA. In some embodiments, the miRNA or the siRNA comprises a polynucleotide sequence that facilitates a reduction of pluripotency of the cell.

In some embodiments, the nucleic acid molecule is chemically modified. In some embodiments, the nucleic acid molecule comprises an unlocked nucleic acid. In some embodiments, the nucleic acid molecule comprises at least two types of nucleic acids. In some embodiments, a molar ratio of polycation amino groups of the polyplex to nucleic acid phosphate groups of the polyplex is at least about 1. In some embodiments, a molar ratio of polycation amino groups of the polyplex to nucleic acid phosphate groups of the polyplex is at most about 60.

Provided herein, are methods for facilitating a change in protein expression in a cell. In an aspect, a method for facilitating a change in protein expression in a cell comprises contacting the cell with a composition comprising: (i) a saccharide, and (ii) a nucleic acid molecule comprising an RNA; under conditions sufficient for the cell to uptake the composition, thereby facilitating the change in the protein expression in the cell.

Provided herein, are methods for facilitating mesodermal, endodermal and ectodermal lineages as follows: adipogenic, angiogenic, cardiogenic, chondrogenic, endothelial, epithelial, hematopoietic, hepatogenic, myogenic, neurogenic, osteogenic, parenchymal, renal, or retinal differentiation of a cell comprises contacting the cell with a composition comprising: (i) a saccharide, and (ii) a nucleic acid molecule comprising an mRNA; under conditions sufficient for the cell to uptake the composition, thereby facilitating the adipogenic, angiogenic, cardiac, cementogenic, chondrogenic, endothelial, epithelial, hematopoietic, hepatogenic, myogenic, neurogenic, osteogenic, or retinal differentiation of the cell.

In some embodiments, change in protein expression facilitates mesodermal, endodermal and ectodermal lineages as follows: adipogenic, angiogenic, cardiogenic, chondrogenic, endothelial, epithelial, hematopoietic, hepatogenic, myogenic, neurogenic, osteogenic, parenchymal, renal, or retinal differentiation of the cell. In some embodiments, the differentiation comprises transdifferentiation of the cell or directed differentiation of the cell. In some embodiments, the cell comprises a somatic cell or a naive cell. In some embodiments, the cell comprises a fat cell, a blood vessel cell, a cardiac cell, a chondrocyte, an endothelial cell, an epithelial cell, a hematopoietic cell, a hepatocyte, a muscle cell, a neuron, or an osteogenic cell. In some embodiments, the cell comprises the muscle cell. In some embodiments, the muscle cell comprises a myofiber, a myotube, a myocyte, a myoblast, a myogenic spheroid, or a muscle cell progenitor. In some embodiments, the cell comprises an animal cell. In some embodiments, the animal cell comprises a mammalian cell, a bird cell, a fish cell, a mollusk cell, or an amphibian cell. In some embodiments, the mammalian cell comprises a porcine cell, a bovine cell, a bubaline cell, an ovine cell, a caprine cell, a cervine cell, a bisontine cell, a cameline cell, an elaphine cell, or a lapine cell. In some embodiments, the bird cell comprises an anatine cell, a galline cell, an anserine cell, a meleagrine cell, a struthionine cell, or a phasianine cell. In some embodiments, the RNA comprises the mRNA. In some embodiments, the RNA encodes MYOD1, MYOG, MYF5, MYF6, PAX3, PAX7, PPARY, adiponectin, FATP1-6, FABP4, GLUT4, Leptin, AdipoR1-2, CD137, a fragment thereof, or a variant thereof.

In some embodiments, the composition further comprises a lipid, an anionic polymer, or a combination thereof. In some embodiments, the RNA is monocistronic or polycistronic. In some embodiments, the RNA comprises an RNA-regulatory element. In some embodiments, the RNA-regulatory element comprises a transcriptional regulatory element, a co-transcriptional regulatory element, a post-transcriptional regulatory element, a translational regulatory element, or any combination thereof. In some embodiments, the RNA-regulatory element comprises a 5′-cap, a 5′-untranslated region (UTR), a 3′-UTR, a poly-A tail modification, or any combination thereof. In some embodiments, the RNA comprises ribonucleic acid (miRNA), transfer ribonucleic acid (tRNA), silencing ribonucleic acid (siRNA), self-amplifying RNA (saRNA), endless/circular RNA (eRNA), trans-amplifying RNA (ta-RNA), or a combination thereof. In some embodiments, the miRNA or the siRNA comprises a polynucleotide sequence that facilitates a reduction of pluripotency of the cell. In some embodiments, the polynucleotide sequence comprises a polynucleotide sequence that comprises POUF51 (OCT3/4), KLF4, or SOX2, or a complementary sequence thereof. In some embodiments, the saccharide is configured to be cationic in an aqueous solution. In some embodiments, the saccharide is configured to be cationic in a neutral aqueous solution. In some embodiments, a cationic charge of cationic moieties of the saccharide comprises at least about 0.5 mequivalent of basic group per gram of polymer (mequiv/g). In some embodiments, a cationic charge of cationic moieties of the saccharide comprises at most about 20 mequiv/g. In some embodiments, the saccharide comprises an average molecular mass of at least about 20 kilodaltons (kDa). In some embodiments, the saccharide comprises an average molecular mass of at most about 2000 kDa. In some embodiments, the saccharide comprises a polysaccharide. In some embodiments, the saccharide and the RNA are configured to self-assemble with each other. In some embodiments, the saccharide and the RNA are configured to form a polyplex. In some embodiments, the RNA is configured to be adsorbed to a surface of the polyplex. In some embodiments, the RNA is configured to be encapsulated within the polyplex. In some embodiments, the polyplex is regularly shaped, irregularly shaped, or branched. In some embodiments, the polyplex is spherical or linear. In some embodiments, an apparent diameter of the polyplex is at least about 5 nanometer (nm), as measured by dynamic light scattering (DLS) or microscopy. In some embodiments, an apparent diameter of the polyplex is at most about 5000 nm, as measured by DLS or microscopy. In some embodiments, the polyplex comprises a nanoparticle. In some embodiments, the saccharide is configured to stabilize the RNA. In some embodiments, the saccharide is configured to inhibit or reduce degradation of the RNA. In some embodiments, the saccharide is configured to inhibit a or reduce nuclease degradation of the RNA.

In some embodiments, the RNA is chemically modified. In some embodiments, the RNA comprises an unlocked nucleic acid. In some embodiments, the RNA comprises at least two types of nucleic acids. In some embodiments, a molar ratio of the saccharide to the RNA is at least about 1. In some embodiments, a molar ratio of the saccharide to the RNA is at most about 60.

Provided herein, are methods for facilitating a change in protein expression in a cell. In an aspect, a method for facilitating a change in protein expression in a cell comprises contacting the stem cell with a composition comprising: (i) a saccharide, and (ii) a nucleic acid molecule; under conditions sufficient for the stem to uptake the composition, wherein the stem cell comprises an embryonic stem cell, an immortalized stem cell, a mesenchymal stem cell, a muscle progenitor cell, or an induced pluripotent stem cell (iPSC), thereby facilitating the change in the protein expression in the stem cell.

Provided herein, are methods for differentiating an induced pluripotent stem cell (iPSC). In an aspect, a method for facilitating a change in protein expression in an iPSC comprises contacting the iPSC with a composition comprising: (i) a saccharide, and (ii) a nucleic acid molecule; under conditions sufficient for the iPSC to uptake the composition, thereby facilitating differentiation of the iPSC.

In some embodiments, the saccharide and the nucleic acid molecule collectively facilitate the change in the protein expression in the stem cell. In some embodiments, the change in the protein expression in the stem cell facilitates differentiation of the stem cell. In some embodiments, the stem cell comprises an animal stem cell. In some embodiments, the animal stem cell comprises a mammalian stem cell, a bird stem cell, a fish stem cell, a mollusk stem cell, or an amphibian stem cell. In some embodiments, the mammalian stem cell comprises a porcine stem cell, a bovine stem cell, a bubaline stem cell, an ovine stem cell, a caprine stem cell, a cervine stem cell, a bisontine stem cell, a cameline stem cell, an elaphine stem cell, or a lapine stem cell. In some embodiments, the bird stem cell comprises an anatine stem cell, a galline stem cell, an anserine stem cell, a meleagrine stem cell, a struthionine stem cell, or a phasianine stem cell. In some embodiments, the composition further comprises a lipid, an anionic polymer, or a combination thereof.

In some embodiments, the saccharide is configured to be cationic in an aqueous solution. In some embodiments, a cationic charge of cationic moieties of the saccharide comprises at least about 0.5 mequivalent of basic group per gram of polymer (mequiv/g). In some embodiments, a cationic charge of cationic moieties of the saccharide comprises at most about 20 mequiv/g. In some embodiments, the saccharide comprises an average molecular mass of at least about 20 kilodaltons (kDa). In some embodiments, the saccharide comprises an average molecular mass of at most about 2000 kDa. In some embodiments, the saccharide comprises a polysaccharide. In some embodiments, the saccharide and the nucleic acid molecule are configured to self-assemble with each other. In some embodiments, the saccharide and the nucleic acid molecule are configured to form a polyplex. In some embodiments, the nucleic acid molecule is configured to be adsorbed to a surface of the polyplex. In some embodiments, the nucleic acid molecule is configured to be encapsulated within the polyplex. In some embodiments, the polyplex is regularly shaped, irregularly shaped, or branched. In some embodiments, the polyplex is spherical or linear. In some embodiments, an apparent diameter of the polyplex is at least about 5 nanometer (nm), as measured by dynamic light scattering (DLS) or microscopy. In some embodiments, an apparent diameter of the polyplex is at most about 5000 nm, as measured by DLS or microscopy. In some embodiments, the polyplex comprises a nanoparticle. In some embodiments, the saccharide is configured to stabilize the nucleic acid molecule. In some embodiments, the saccharide is configured to inhibit or reduce degradation of the nucleic acid molecule. In some embodiments, the saccharide is configured to inhibit a or reduce nuclease degradation of the nucleic acid molecule.

In some embodiments, the nucleic acid molecule comprises an RNA. In some embodiments, the RNA comprises, micro ribonucleic acid (miRNA), transfer ribonucleic acid (tRNA), silencing ribonucleic acid (siRNA), self-amplifying RNA (saRNA), endless/circular RNA (eRNA), trans-amplifying RNA (ta-RNA), or a combination thereof. In some embodiments, the RNA is monocistronic or polycistronic. In some embodiments, the RNA comprises an RNA-regulatory element. In some embodiments, the RNA-regulatory element comprises a transcriptional regulatory element, a co-transcriptional regulatory element, a post-transcriptional regulatory element, a translational regulatory element, or any combination thereof. In some embodiments, the RNA-regulatory element comprises a 5′-cap, 5′ UTRs, a 3′-untranslated region (UTR), a poly-A tail modification, or any combination thereof. In some embodiments, the nucleic acid molecule comprises miRNA or the siRNA. In some embodiments, the miRNA or the siRNA comprises a polynucleotide sequence that facilitates a reduction of pluripotency of the stem cell.

In some embodiments, the nucleic acid molecule is chemically modified. In some embodiments, the nucleic acid molecule comprises an unlocked nucleic acid. In some embodiments, the nucleic acid molecule comprises at least two types of nucleic acids. In some embodiments, a molar ratio of polycation amino groups of the polyplex to nucleic acid phosphate groups of the polyplex is at least about 1 or at most about 60. In some embodiments, the nucleic acid molecule comprises messenger ribonucleic acid (mRNA).

Provided herein, are methods for facilitating a change in protein expression in a cell. In an aspect, a method for facilitating a change in protein expression in a cell comprises contacting the cell with a composition comprising: (i) a modified saccharide comprising an aliphatic aldehyde modified saccharide, an aromatic aldehyde modified saccharide, a quaternary ammonium modified saccharide, a polyamine derivatized saccharide, a saccharide modified saccharide, a saccharide with a lipid additive, or a combination thereof, and (ii) a nucleic acid molecule comprising an mRNA; under conditions sufficient for the cell to uptake the composition, thereby facilitating the change in the protein expression in the cell.

Provided herein, are methods for facilitating differentiation of a cell. In an aspect, a method for facilitating differentiation of a cell comprises contacting the cell with a composition comprising: (i) a modified saccharide comprising an aliphatic aldehyde modified saccharide, an aromatic aldehyde modified saccharide, a quaternary ammonium modified saccharide, a polyamine derivatized saccharide, a saccharide modified saccharide, a saccharide with a lipid additive, or a combination thereof, and (ii) a nucleic acid molecule comprising an mRNA; under conditions sufficient for the cell to uptake the composition, thereby facilitating the differentiation of the cell.

In some embodiments, the modified saccharide comprises a chitosan, a hyaluronic acid, a pullulan, a heparin, an alginate, or a combination or derivative thereof. In some embodiments, the modified saccharide comprises a modified chitosan. In some embodiments, the modified saccharide comprises an aliphatic aldehyde modified saccharide, an aromatic aldehyde modified saccharide, a quaternary ammonium modified saccharide, a polyamine derivatized saccharide, a saccharide modified saccharide, a lipid modified saccharide, a phenol modified saccharide, or a combination thereof. In some embodiments, the aliphatic aldehyde modified saccharide comprises a hexanal aldehyde modified saccharide, a heptanal aldehyde modified saccharide, an octanal aldehyde modified saccharide, a nonanal aldehyde modified saccharide, or a decanal aldehyde modified saccharide. In some embodiments, the aliphatic aldehyde modified saccharide comprises a decanal aldehyde modified saccharide. In some embodiments, the aromatic aldehyde modified saccharide comprises a benzaldehyde modified saccharide or a cinnamaldehyde modified saccharide. In some embodiments, the polyamine derivatized saccharide comprises a spermine modified saccharide, a spermidine modified saccharide, a putrescine modified saccharide, a diethylethylamine modified saccharide, a dimethylethylamine modified saccharide, a quaternary ammonium modified saccharide, or an arginine modified saccharide. In some embodiments, the saccharide with the lipid additive comprises a saccharide with a lecithin or a saccharide with a phosphatidylcholine. In some embodiments, the saccharide modified saccharide comprises a monosaccharide modified saccharide or a polysaccharide modified saccharide. In some embodiments, the monosaccharide modified saccharide comprises a lactose modified saccharide, a mannose modified saccharide, a glucose modified saccharide, a galactose modified saccharide, a glucosamine modified saccharide, a sucrose modified saccharide, a maltose modified saccharide, a xylose modified saccharide, a ribose modified saccharide, a fructose modified saccharide, or a glyceraldehyde modified saccharide. In some embodiments, the polysaccharide modified saccharide comprises an anionic polysaccharide modified saccharide or a reducing polysaccharide modified saccharide. In some embodiments, the anionic polysaccharide modified saccharide comprises an alginate modified saccharide, a carboxymethylated cellulose modified saccharide, a hyaluronic acid modified saccharide, a pectin modified saccharide, a pullulan modified saccharide, a starch modified saccharide, or a xanthan gum modified saccharide. In some embodiments, the reducing polysaccharide modified saccharide comprises a maltodextrin modified saccharide or a cellobiose modified saccharide. In some embodiments, the reducing polysaccharide modified saccharide comprises a cationic lipid additive. In some embodiments, the phenol modified saccharide comprises a chlorogenic acid modified saccharide, a ferulic acid modified saccharide, a caffeic acid modified saccharide, a gallic acid modified saccharide, or a combination thereof.

In some embodiments, the change in the protein expression in the cell facilitates differentiation of the cell. In some embodiments, the modified saccharide is configured to be cationic in an aqueous solution. In some embodiments, the modified saccharide is configured to be cationic in a neutral aqueous solution. In some embodiments, a cationic charge of cationic moieties of the saccharide comprises at least about 0.5 mequivalent of basic group per gram of polymer (mequiv/g). In some embodiments, a cationic charge of cationic moieties of the saccharide comprises at most about 20 mequiv/g. In some embodiments, the modified saccharide comprises an average molecular mass of at least about 20 kilodaltons (kDa). In some embodiments, the modified saccharide comprises an average molecular mass of at most about 2000 kDa. In some embodiments, the modified saccharide comprises a modified polysaccharide.

In some embodiments, the modified saccharide and the mRNA are configured to self-assemble with each other. In some embodiments, the modified saccharide and the mRNA are configured to form a polyplex. In some embodiments, the mRNA is configured to be adsorbed to a surface of the polyplex. In some embodiments, the mRNA is configured to be encapsulated within the polyplex. In some embodiments, the polyplex is regularly shaped, irregularly shaped, or branched. In some embodiments, the polyplex is spherical or linear. In some embodiments, an apparent diameter of the polyplex is at least about 5 nanometer (nm), as measured by dynamic light scattering (DLS) or microscopy. In some embodiments, an apparent diameter of the polyplex is at most about 5000 nm, as measured by DLS or microscopy. In some embodiments, the polyplex comprises a nanoparticle. In some embodiments, the modified saccharide is configured to stabilize the mRNA. In some embodiments, the modified saccharide is configured to inhibit or reduce degradation of the mRNA. In some embodiments, the modified saccharide is configured to inhibit a or reduce nuclease degradation of the mRNA.

In some embodiments, the mRNA is monocistronic or polycistronic. In some embodiments, the mRNA comprises a non-coding sequence. In some embodiments, the mRNA comprises an RNA-regulatory element. In some embodiments, the RNA-regulatory element comprises a transcriptional regulatory element, a co-transcriptional regulatory element, a post-transcriptional regulatory element, a translational regulatory element, or any combination thereof. In some embodiments, the RNA-regulatory element comprises a 5′-cap, a 5′-untranslated region (UTR), a 3′-UTR, a poly-A tail modification, or any combination thereof. In some embodiments, the composition further comprises micro ribonucleic acid (miRNA), transfer ribonucleic acid (tRNA), silencing ribonucleic acid (siRNA), self-amplifying RNA (saRNA), endless/circular RNA (eRNA), trans-amplifying RNA (ta-RNA), or a combination thereof. In some embodiments, the composition further comprises the miRNA or the siRNA. In some embodiments, the miRNA or the siRNA comprises a polynucleotide sequence that facilitates a reduction of pluripotency of the cell.

In some embodiments, the mRNA is chemically modified. In some embodiments, the mRNA comprises an unlocked nucleic acid. In some embodiments, the mRNA comprises at least two types of nucleic acids. In some embodiments, a molar ratio of the saccharide to the mRNA is at least about 1. In some embodiments, a molar ratio of the saccharide to the mRNA is at most about 60.

Provided herein, are edible products prepared by processes. In an aspect, an edible product prepared by a process comprises contacting a cell with a composition comprising a saccharide or a nucleic acid molecule, which the saccharide or the nucleic acid molecule are configured to facilitate a change in protein expression in the cell.

In another aspect, an edible product prepared by a process comprises contacting a cell with a composition comprising a saccharide or a nucleic acid molecule, which the saccharide or the nucleic acid molecule are configured to facilitate differentiation of the cell.

In some embodiments, the change in the protein expression in the cell facilitates differentiation of the cell. In some embodiments, the saccharide is configured to be cationic in an aqueous solution. In some embodiments, the composition further comprises a lipid, an anionic polymer, or a combination thereof. In some embodiments, a cationic charge of cationic moieties of the saccharide comprises at least about 0.5 mequivalent of basic group per gram of polymer (mequiv/g). In some embodiments, a cationic charge of cationic moieties of the saccharide comprises at most about 20 mequiv/g. In some embodiments, the saccharide comprises an average molecular mass of at least about 20 kilodaltons (kDa). In some embodiments, the saccharide comprises an average molecular mass of at most about 2000 kDa. In some embodiments, the saccharide comprises a polysaccharide. In some embodiments, the saccharide and the nucleic acid molecule are configured to self-assemble with each other. In some embodiments, the saccharide and the nucleic acid molecule are configured to form a polyplex. In some embodiments, the nucleic acid molecule is configured to be adsorbed to a surface of the polyplex. In some embodiments, the nucleic acid molecule is configured to be encapsulated within the polyplex. In some embodiments, the polyplex is regularly shaped, irregularly shaped, or branched. In some embodiments, the polyplex is spherical or linear. In some embodiments, an apparent diameter of the polyplex is at least about 5 nanometer (nm), as measured by dynamic light scattering (DLS) or microscopy. In some embodiments, an apparent diameter of the polyplex is at most about 5000 nm, as measured by DLS or microscopy. In some embodiments, the polyplex comprises a nanoparticle. In some embodiments, the saccharide is configured to stabilize the nucleic acid molecule. In some embodiments, the saccharide is configured to inhibit or reduce degradation of the nucleic acid molecule. In some embodiments, the saccharide is configured to inhibit or reduce nuclease degradation of the nucleic acid molecule.

In some embodiments, the nucleic acid molecule comprises an RNA. In some embodiments, the RNA comprises messenger ribonucleic acid (mRNA), micro ribonucleic acid (miRNA), transfer ribonucleic acid (tRNA), silencing ribonucleic acid (siRNA), self-amplifying RNA (saRNA), endless/circular RNA (eRNA), trans-amplifying RNA (ta-RNA), or a combination thereof. In some embodiments, the RNA is monocistronic or polycistronic. In some embodiments, the RNA comprises an RNA-regulatory element. In some embodiments, the RNA-regulatory element comprises a transcriptional regulatory element, a co-transcriptional regulatory element, a post-transcriptional regulatory element, a translational regulatory element, or any combination thereof. In some embodiments, the RNA-regulatory element comprises a 5′-cap, a 5′-untranslated region (UTR), a 3′-UTR, a poly-A tail modification, or any combination thereof. In some embodiments, the RNA comprises the mRNA. In some embodiments, the miRNA or the siRNA comprises a polynucleotide sequence that facilitates a reduction of pluripotency of the cell.

In some embodiments, the nucleic acid molecule is chemically modified. In some embodiments, the nucleic acid molecule comprises an unlocked nucleic acid. In some embodiments, the nucleic acid molecule comprises at least two types of nucleic acids. In some embodiments, a molar ratio of polycation amino groups of the polyplex to nucleic acid phosphate groups of the polyplex is at least about 1 or at most about 60. In some embodiments, the saccharide and the nucleic acid molecule are configured to collectively facilitate a change in protein expression in the cell.

Provided herein, are pharmaceutical active ingredients prepared by processes. In an aspect, a pharmaceutical active ingredient prepared by a process comprises using a saccharide and a nucleic acid molecule, which the saccharide or the nucleic acid molecule are configured to collectively facilitate a change in protein expression in a cell.

In another aspect, a pharmaceutical active ingredient prepared by a process comprises using a saccharide and a nucleic acid molecule, which the saccharide or the nucleic acid molecule are configured to collectively facilitate differentiation of a cell.

In some embodiments, the pharmaceutical active ingredient is formulated as a drug substance, a drug product, or a medicine. In some embodiments, the change in the protein expression in the cell facilitates differentiation of the cell. In some embodiments, the saccharide is configured to be cationic in an aqueous solution. In some embodiments, the process further comprises using a lipid, an anionic polymer, or a combination thereof. In some embodiments, a cationic charge of cationic moieties of the saccharide comprises at least about 0.5 mequivalent of basic group per gram of polymer (mequiv/g). In some embodiments, a cationic charge of cationic moieties of the saccharide comprises at most about 20 mequiv/g. In some embodiments, the saccharide comprises an average molecular mass of at least about 20 kilodaltons (kDa). In some embodiments, the saccharide comprises an average molecular mass of at most about 2000 kDa. In some embodiments, the saccharide comprises a polysaccharide. In some embodiments, the saccharide and the nucleic acid molecule are configured to self-assemble with each other. In some embodiments, the saccharide and the nucleic acid molecule are configured to form a polyplex. In some embodiments, the nucleic acid molecule is configured to be adsorbed to a surface of the polyplex. In some embodiments, the nucleic acid molecule is configured to be encapsulated within the polyplex. In some embodiments, the polyplex is regularly shaped, irregularly shaped, or branched. In some embodiments, the polyplex is spherical or linear. In some embodiments, an apparent diameter of the polyplex is at least about 5 nanometer (nm), as measured by dynamic light scattering (DLS) or microscopy. In some embodiments, an apparent diameter of the polyplex is at most about 5000 nm, as measured by DLS or microscopy. In some embodiments, the polyplex comprises a nanoparticle. In some embodiments, the saccharide is configured to stabilize the nucleic acid molecule. In some embodiments, the saccharide is configured to inhibit or reduce degradation of the nucleic acid molecule. In some embodiments, the saccharide is configured to inhibit or reduce nuclease degradation of the nucleic acid molecule.

In some embodiments, the nucleic acid molecule comprises an RNA. In some embodiments, the RNA comprises messenger ribonucleic acid (mRNA), micro ribonucleic acid (miRNA), transfer ribonucleic acid (tRNA), silencing ribonucleic acid (siRNA), self-amplifying RNA (saRNA), endless/circular RNA (eRNA), trans-amplifying RNA (ta-RNA), or a combination thereof. In some embodiments, the RNA is monocistronic or polycistronic. In some embodiments, the RNA comprises an RNA-regulatory element. In some embodiments, the RNA-regulatory element comprises a transcriptional regulatory element, a co-transcriptional regulatory element, a post-transcriptional regulatory element, a translational regulatory element, or any combination thereof. In some embodiments, the RNA-regulatory element comprises a 5′-cap, a 5′-untranslated region (UTR), a 3′-UTR, a poly-A tail modification, or any combination thereof. In some embodiments, the RNA comprises the mRNA. In some embodiments, the miRNA or the siRNA comprises a polynucleotide sequence that facilitates a reduction of pluripotency of the cell. In some embodiments, the nucleic acid molecule is chemically modified. In some embodiments, the nucleic acid molecule comprises an unlocked nucleic acid. In some embodiments, the nucleic acid molecule comprises at least two types of nucleic acids. In some embodiments, a molar ratio of polycation amino groups of the polyplex to nucleic acid phosphate groups of the polyplex is at least about 1 or at most about 60. In some embodiments, the saccharide and the nucleic acid molecule are configured to collectively facilitate the change in protein expression in the cell.

Provided herein, are pharmaceutical active ingredients prepared by processes. In an aspect, a pharmaceutical active ingredient prepared by a process comprises using a saccharide and a nucleic acid molecule, wherein the saccharide comprising an aliphatic aldehyde modified saccharide, an aromatic aldehyde modified saccharide, a quaternary ammonium modified saccharide, a polyamine derivatized saccharide, a saccharide modified saccharide, a saccharide with a lipid additive, or a combination thereof; wherein the nucleic acid or the modified saccharide is configured to facilitate a change in protein expression in a cell.

Provided herein, are pharmaceutical active ingredients prepared by processes. In an aspect, a pharmaceutical active ingredient prepared by a process comprises using a saccharide and a nucleic acid molecule, wherein the saccharide comprising an aliphatic aldehyde modified saccharide, an aromatic aldehyde modified saccharide, a quaternary ammonium modified saccharide, a polyamine derivatized saccharide, a saccharide modified saccharide, a saccharide with a lipid additive, or a combination thereof; wherein the nucleic acid or the modified saccharide is configured to facilitate differentiation of a cell.

In some embodiments, the pharmaceutical active ingredient is formulated as a drug substance, a drug product, or a medicine. In some embodiments, the aliphatic aldehyde modified saccharide comprises a hexanal aldehyde modified saccharide, a heptanal aldehyde modified saccharide, an octanal aldehyde modified saccharide, a nonanal aldehyde modified saccharide, or a decanal aldehyde modified saccharide. In some embodiments, the aliphatic aldehyde modified saccharide comprises a decanal aldehyde modified saccharide. In some embodiments, the aromatic aldehyde modified saccharide comprises a benzaldehyde modified saccharide or a cinnamaldehyde modified saccharide. In some embodiments, the polyamine derivatized saccharide comprises a spermine modified saccharide, a spermidine modified saccharide, a putrescine modified saccharide, or an arginine modified saccharide. In some embodiments, the saccharide with the lipid additive comprises a saccharide with a lecithin or a saccharide with a phosphatidylcholine. In some embodiments, the saccharide modified saccharide comprises a monosaccharide modified saccharide or a polysaccharide modified saccharide. In some embodiments, the monosaccharide modified saccharide comprises a lactose modified saccharide, a mannose modified saccharide, a glucose modified saccharide, a galactose modified saccharide, a glucosamine modified saccharide, a sucrose modified saccharide, a maltose modified saccharide, a xylose modified saccharide, a ribose modified saccharide, a fructose modified saccharide, or a glyceraldehyde modified saccharide. In some embodiments, the polysaccharide modified saccharide comprises an anionic polysaccharide modified saccharide or a reducing polysaccharide modified saccharide. In some embodiments, the anionic polysaccharide modified saccharide comprises an alginate modified saccharide, a carboxymethylated cellulose modified saccharide, a hyaluronic acid modified saccharide, a pectin modified saccharide, a pullulan modified saccharide, a starch modified saccharide, or a xanthan gum modified saccharide. In some embodiments, the reducing polysaccharide modified saccharide comprises a maltodextrin modified saccharide or a cellobiose modified saccharide. In some embodiments, the reducing polysaccharide modified saccharide comprises a cationic lipid additive. In some embodiments, the phenol modified saccharide comprises a chlorogenic acid modified saccharide, a ferulic acid modified saccharide, a caffeic acid modified saccharide, a gallic acid modified saccharide, or a combination thereof.

In some embodiments, the saccharide or the nucleic acid molecule are configured to collectively facilitate the change in the protein expression in the cell. In some embodiments, the saccharide is configured to be cationic in an aqueous solution. In some embodiments, a cationic charge of cationic moieties of the saccharide comprises at least about 0.5 mequivalent of basic group per gram of polymer (mequiv/g). In some embodiments, a cationic charge of cationic moieties of the saccharide comprises at most about 20 mequiv/g. In some embodiments, the saccharide comprises an average molecular mass of at least about 20 kilodaltons (kDa). In some embodiments, the saccharide comprises an average molecular mass of at most about 2000 kDa. In some embodiments, the saccharide comprises a polysaccharide. In some embodiments, the saccharide and the nucleic acid molecule are configured to self-assemble with each other. In some embodiments, the saccharide and the nucleic acid molecule are configured to form a polyplex. In some embodiments, the nucleic acid molecule is configured to be adsorbed to a surface of the polyplex. In some embodiments, the nucleic acid molecule is configured to be encapsulated within the polyplex. In some embodiments, the polyplex is regularly shaped, irregularly shaped, or branched. In some embodiments, the polyplex is spherical or linear. In some embodiments, an apparent diameter of the polyplex is at least about 5 nanometer (nm), as measured by dynamic light scattering (DLS) or microscopy. In some embodiments, an apparent diameter of the polyplex is at most about 5000 nm, as measured by DLS or microscopy. In some embodiments, the polyplex comprises a nanoparticle. In some embodiments, the saccharide is configured to stabilize the nucleic acid molecule. In some embodiments, the saccharide is configured to inhibit or reduce degradation of the nucleic acid molecule. In some embodiments, the saccharide is configured to inhibit or reduce nuclease degradation of the nucleic acid molecule.

In some embodiments, the nucleic acid molecule comprises an RNA. In some embodiments, the RNA comprises messenger ribonucleic acid (mRNA), micro ribonucleic acid (miRNA), transfer ribonucleic acid (tRNA), silencing ribonucleic acid (siRNA), self-amplifying RNA (saRNA), endless/circular RNA (eRNA), trans-amplifying RNA (ta-RNA), or a combination thereof. In some embodiments, the RNA is monocistronic or polycistronic. In some embodiments, the RNA comprises an RNA-regulatory element. In some embodiments, the RNA-regulatory element comprises a transcriptional regulatory element, a co-transcriptional regulatory element, a post-transcriptional regulatory element, a translational regulatory element, or any combination thereof. In some embodiments, the RNA-regulatory element comprises a 5′-cap, a 5′-untranslated region (UTR), a 3′-UTR, a poly-A tail modification, or any combination thereof. In some embodiments, the RNA comprises the mRNA. In some embodiments, the miRNA or the siRNA comprises a polynucleotide sequence that facilitates a reduction of pluripotency of the cell. In some embodiments, the nucleic acid molecule is chemically modified. In some embodiments, the nucleic acid molecule comprises an unlocked nucleic acid. In some embodiments, the nucleic acid molecule comprises at least two types of nucleic acids. In some embodiments, a molar ratio of polycation amino groups of the polyplex to nucleic acid phosphate groups of the polyplex is at least about 1. In some embodiments, a molar ratio of polycation amino groups of the polyplex to nucleic acid phosphate groups of the polyplex is at most about 60.

Provided herein, are tissues prepared by processes. In an aspect, a tissue prepared by a process comprises using a saccharide and a nucleic acid molecule, which the saccharide or the nucleic acid molecule is configured to facilitate a change in protein expression in a cell.

In another aspect, a tissue prepared by a process comprises using a saccharide and a nucleic acid molecule, which the saccharide or the nucleic acid molecule is configured to facilitate differentiation of a cell.

In some embodiments, the tissue comprises an organ, an organoid, or a model thereof. In some embodiments, the model comprises a biomimetic model of the tissue, the organ, or the organoid.

In some embodiments, the change in the protein expression in the cell facilitates differentiation of the cell. In some embodiments, the process further comprises using a lipid, an anionic polymer, or a combination thereof. In some embodiments, a cationic charge of cationic moieties of the saccharide comprises at least about 0.5 mequivalent of basic group per gram of polymer (mequiv/g). In some embodiments, a cationic charge of cationic moieties of the saccharide comprises at most about 20 mequiv/g. In some embodiments, the saccharide comprises an average molecular mass of at least about 20 kilodaltons (kDa). In some embodiments, the saccharide comprises an average molecular mass of at most about 2000 kDa. In some embodiments, the saccharide comprises a polysaccharide. In some embodiments, the saccharide and the nucleic acid molecule are configured to self-assemble with each other. In some embodiments, the saccharide and the nucleic acid molecule are configured to form a polyplex. In some embodiments, the nucleic acid molecule is configured to be adsorbed to a surface of the polyplex. In some embodiments, the nucleic acid molecule is configured to be encapsulated within the polyplex. In some embodiments, the polyplex is regularly shaped, irregularly shaped, or branched. In some embodiments, the polyplex is spherical or linear. In some embodiments, an apparent diameter of the polyplex is at least about 5 nanometer (nm), as measured by dynamic light scattering (DLS) or microscopy. In some embodiments, an apparent diameter of the polyplex is at most about 5000 nm, as measured by DLS or microscopy. In some embodiments, the polyplex comprises a nanoparticle. In some embodiments, the saccharide is configured to stabilize the nucleic acid molecule. In some embodiments, the saccharide is configured to inhibit or reduce degradation of the nucleic acid molecule. In some embodiments, the saccharide is configured to inhibit or reduce nuclease degradation of the nucleic acid molecule.

In some embodiments, the nucleic acid molecule comprises an RNA. In some embodiments, the RNA comprises messenger ribonucleic acid (mRNA), micro ribonucleic acid (miRNA), transfer ribonucleic acid (tRNA), silencing ribonucleic acid (siRNA), self-amplifying RNA (saRNA), endless/circular RNA (eRNA), trans-amplifying RNA (ta-RNA), or a combination thereof. In some embodiments, the RNA is monocistronic or polycistronic. In some embodiments, the RNA comprises an RNA-regulatory element. In some embodiments, the RNA-regulatory element comprises a transcriptional regulatory element, a co-transcriptional regulatory element, a post-transcriptional regulatory element, a translational regulatory element, or any combination thereof. In some embodiments, the RNA-regulatory element comprises a 5′-cap, a 5′-untranslated region (UTR), a 3′-UTR, a poly-A tail modification, or any combination thereof. In some embodiments, the RNA comprises the mRNA. In some embodiments, the miRNA or the siRNA comprises a polynucleotide sequence that facilitates a reduction of pluripotency of the cell. In some embodiments, the nucleic acid molecule is chemically modified. In some embodiments, the nucleic acid molecule comprises an unlocked nucleic acid. In some embodiments, the nucleic acid molecule comprises at least two types of nucleic acids. In some embodiments, a molar ratio of polycation amino groups of the polyplex to nucleic acid phosphate groups of the polyplex is at least about 1 or at most about 60. In some embodiments, the saccharide and the nucleic acid molecule are configured to collectively facilitate the change in protein expression in the cell.

Provided herein, are pharmaceutical active ingredients prepared by processes. In an aspect, a pharmaceutical active ingredient prepared by a process comprises using a saccharide and a nucleic acid molecule, wherein the saccharide comprising an aliphatic aldehyde modified saccharide, an aromatic aldehyde modified saccharide, a quaternary ammonium modified saccharide, a polyamine derivatized saccharide, a saccharide modified saccharide, a saccharide with a lipid additive, or a combination thereof; wherein the nucleic acid or the modified saccharide is configured to facilitate a change in protein expression in a cell.

In another aspect, a pharmaceutical active ingredient prepared by a process comprises using a saccharide and a nucleic acid molecule, wherein the saccharide comprising an aliphatic aldehyde modified saccharide, an aromatic aldehyde modified saccharide, a quaternary ammonium modified saccharide, a polyamine derivatized saccharide, a saccharide modified saccharide, a saccharide with a lipid additive, or a combination thereof; wherein the nucleic acid or the modified saccharide is configured to facilitate differentiation of a cell.

In some embodiments, the tissue comprises an organ, an organoid, or a model thereof. In some embodiments, the aliphatic aldehyde modified saccharide comprises a hexanal aldehyde modified saccharide, a heptanal aldehyde modified saccharide, an octanal aldehyde modified saccharide, a nonanal aldehyde modified saccharide, or a decanal aldehyde modified saccharide. In some embodiments, the aliphatic aldehyde modified saccharide comprises a decanal aldehyde modified saccharide. In some embodiments, the aromatic aldehyde modified saccharide comprises a benzaldehyde modified saccharide or a cinnamaldehyde modified saccharide. In some embodiments, the polyamine derivatized saccharide comprises a spermine modified saccharide, a spermidine modified saccharide, a putrescine modified saccharide, or an arginine modified saccharide. In some embodiments, the saccharide with the lipid additive comprises a saccharide with a lecithin or a saccharide with a phosphatidylcholine. In some embodiments, the saccharide modified saccharide comprises a monosaccharide modified saccharide or a polysaccharide modified saccharide. In some embodiments, the monosaccharide modified saccharide comprises a lactose modified saccharide, a mannose modified saccharide, a glucose modified saccharide, a galactose modified saccharide, a glucosamine modified saccharide, a sucrose modified saccharide, a maltose modified saccharide, a xylose modified saccharide, a ribose modified saccharide, a fructose modified saccharide, or a glyceraldehyde modified saccharide. In some embodiments, the polysaccharide modified saccharide comprises an anionic polysaccharide modified saccharide or a reducing polysaccharide modified saccharide. In some embodiments, the anionic polysaccharide modified saccharide comprises an alginate modified saccharide, a carboxymethylated cellulose modified saccharide, a hyaluronic acid modified saccharide, a pectin modified saccharide, a pullulan modified saccharide, a starch modified saccharide, or a xanthan gum modified saccharide. In some embodiments, the reducing polysaccharide modified saccharide comprises a maltodextrin modified saccharide or a cellobiose modified saccharide. In some embodiments, the reducing polysaccharide modified saccharide comprises a cationic lipid additive. In some embodiments, the phenol modified saccharide comprises a chlorogenic acid modified saccharide, a ferulic acid modified saccharide, a caffeic acid modified saccharide, a gallic acid modified saccharide, or a combination thereof. In some embodiments, the model comprises a biomimetic model of the tissue, the organ, or the organoid.

In some embodiments, the saccharide and the nucleic acid molecule are configured to collectively facilitate the change in the protein expression in a cell. In some embodiments, a cationic charge of cationic moieties of the saccharide comprises at least about 0.5 mequivalent of basic group per gram of polymer (mequiv/g). In some embodiments, a cationic charge of cationic moieties of the saccharide comprises at most about 20 mequiv/g. In some embodiments, the saccharide comprises an average molecular mass of at least about 20 kilodaltons (kDa). In some embodiments, the saccharide comprises an average molecular mass of at most about 2000 kDa. In some embodiments, the saccharide comprises a polysaccharide. In some embodiments, the saccharide and the nucleic acid molecule are configured to self-assemble with each other. In some embodiments, the saccharide and the nucleic acid molecule are configured to form a polyplex. In some embodiments, the nucleic acid molecule is configured to be adsorbed to a surface of the polyplex. In some embodiments, the nucleic acid molecule is configured to be encapsulated within the polyplex. In some embodiments, the polyplex is regularly shaped, irregularly shaped, or branched. In some embodiments, the polyplex is spherical or linear. In some embodiments, an apparent diameter of the polyplex is at least about 5 nanometer (nm), as measured by dynamic light scattering (DLS) or microscopy. In some embodiments, an apparent diameter of the polyplex is at most about 5000 nm, as measured by DLS or microscopy. In some embodiments, the polyplex comprises a nanoparticle. In some embodiments, the saccharide is configured to stabilize the nucleic acid molecule. In some embodiments, the saccharide is configured to inhibit or reduce degradation of the nucleic acid molecule. In some embodiments, the saccharide is configured to inhibit or reduce nuclease degradation of the nucleic acid molecule.

In some embodiments, the nucleic acid molecule comprises an RNA. In some embodiments, the RNA comprises messenger ribonucleic acid (mRNA), micro ribonucleic acid (miRNA), transfer ribonucleic acid (tRNA), silencing ribonucleic acid (siRNA), self-amplifying RNA (saRNA), endless/circular RNA (eRNA), trans-amplifying RNA (ta-RNA), or a combination thereof. In some embodiments, the RNA is monocistronic or polycistronic. In some embodiments, the RNA comprises an RNA-regulatory element. In some embodiments, the RNA-regulatory element comprises a transcriptional regulatory element, a co-transcriptional regulatory element, a post-transcriptional regulatory element, a translational regulatory element, or any combination thereof. In some embodiments, the RNA-regulatory element comprises a 5′-cap, a 5′-untranslated region (UTR), a 3′-UTR, a poly-A tail modification, or any combination thereof. In some embodiments, the RNA comprises the mRNA. In some embodiments, the miRNA or the siRNA comprises a polynucleotide sequence that facilitates a reduction of pluripotency of the cell. In some embodiments, the nucleic acid molecule is chemically modified. In some embodiments, the nucleic acid molecule comprises an unlocked nucleic acid. In some embodiments, the nucleic acid molecule comprises at least two types of nucleic acids. In some embodiments, a molar ratio of polycation amino groups of the polyplex to nucleic acid phosphate groups of the polyplex is at least about 1 or at most about 60. In some embodiments, the saccharide is configured to be cationic in an aqueous solution.

Additional aspects and advantages of the present disclosure will become readily apparent to those skilled in this art from the following detailed description, wherein only illustrative embodiments of the present disclosure are shown and described. As will be realized, the present disclosure is capable of other and different embodiments, and its several details are capable of modifications in various obvious respects, all without departing from the disclosure. Accordingly, the drawings and description are to be regarded as illustrative in nature, and not as restrictive.

### INCORPORATION BY REFERENCE

All publications, patents, and patent applications mentioned in this specification are herein incorporated by reference to the same extent as if each individual publication, patent, or patent application was specifically and individually indicated to be incorporated by reference. To the extent publications and patents or patent applications incorporated by reference contradict the disclosure contained in the specification, the specification is intended to supersede and/or take precedence over any such contradictory material.

### BRIEF DESCRIPTION OF THE DRAWINGS

The novel features of the disclosure are set forth with particularity in the appended claims. A better understanding of the features and advantages of the present disclosure will be obtained by reference to the following detailed description that sets forth illustrative embodiments, in which the principles of the disclosure are utilized, and the accompanying drawings (also “Figure” and “FIG.” herein), of which:

FIGS. 1A-C show cells transfected with nucleic acid molecules encoding green fluorescent protein (GFP). Selected cells show GFP expression.

FIG. 2 shows cells transfected with exemplary compositions comprising saccharides and nucleic acid molecules.

FIG. 3 shows cells transfected with other exemplary compositions comprising saccharides and nucleic acid molecules.

FIG. 4 depicts a schematic workflow for transfecting cells with compositions comprising saccharides and nucleic acid molecules.

### DETAILED DESCRIPTION

While various embodiments of the disclosure have been shown and described herein, it will be obvious to those skilled in the art that such embodiments are provided by way of example only. Numerous variations, changes, and substitutions may occur to those skilled in the art without departing from the disclosure. It should be understood that various alternatives to the embodiments of the disclosure described herein may be employed.

Provided herein, are methods and compositions for altering the protein expression of a cell. Provided herein, are methods and compositions for differentiation of a cell. Provided herein, are methods and compositions for or facilitating the cell to produce a protein or proteins of pharmaceutical and economic importance. The methods may comprise contacting a cell with a composition. In some cases, the composition may comprise a saccharide and a nucleic acid molecule. A cell taking up the nucleic acid may undergo alternations in protein expression that result in differentiation. The efficiency of a cell taking up a nucleic acid molecule using traditional methods may be low. The low efficiency of the cell taking up the nucleic acid may limit the applicability of the methods and compositions for differentiating the cell. The lack of biodegradability of non-viral vectors used in traditional methods may limit the applicability of the methods and compositions for altering and/or changing the protein expression and may limit cell differentiation. The instant disclosure provides methods and compositions for increasing the transfection efficiency, thereby reducing the amount of nucleic acid molecules, cells, or other reagents to transfect the cells with the nucleic acid molecules.

A transfected cell may altered protein or gene expression. A transfected cell may undergo differentiation. A differentiated cell may have a different structural and/or functional characteristic relative to a cell that is not differentiated. In some cases, a cell with a different structural and/or functional characteristic may be used in a wide variety of purposes. In some cases, a cell with a different structural and/or functional characteristic may be edible. In some cases, a cell with a different structural and/or functional characteristic may be used to produce an edible meat product. The compositions provided herein may further comprise a polymeric material. The polymeric materials provided herein may facilitate differentiation of the cell that has contacted with any of the nucleic acid molecules and saccharides provided herein.

A saccharide or lipid of a composition described herein may be biodegradable. Biodegradable saccharides or lipids can be metabolized by various enzymes (e.g., esterase, peptidases) to minimize any toxicity, immunogenicity, and/or carcinogenicity.

### Definitions

The term “about” or “approximately” as used herein when referring to a measurable value such as an amount or concentration and the like, is meant to encompass variations of 20%, 10%, 5%, 1%, 0.5%, or even 0.1% of the specified amount. For example, “about” can mean plus or minus 10%, per the practice in the art. Alternatively, “about” can mean a range of plus or minus 20%, plus or minus 10%, plus or minus 5%, or plus or minus 1% of a given value. Alternatively, particularly with respect to biological systems or processes, the term can mean within an order of magnitude, up to 5-fold, or up to 2-fold, of a value. Where particular values can be described in the application and claims, unless otherwise stated the term “about” may be assumed to encompass the acceptable error range for the particular value. Also, where ranges, subranges, or both, of values can be provided, the ranges or subranges can include the endpoints of the ranges or subranges. The terms “substantially”, “substantially no”, “substantially free”, and “approximately” can be used when describing a magnitude, a position or both to indicate that the value described can be up to a reasonable expected range of values. For example, a numeric value can have a value that can be +/−0.1% of the stated value (or range of values), +/−1% of the stated value (or range of values), +/−2% of the stated value (or range of values), +/−5% of the stated value (or range of values), +/−10% of the stated value (or range of values), etc. Any numerical range recited herein can be intended to include all sub-ranges subsumed therein.

Where values are described as ranges, it may be understood that such disclosure includes the disclosure of all possible sub-ranges within such ranges, as well as specific numerical values that fall within such ranges irrespective of whether a specific numerical value or specific sub-range is expressly stated.

The terms “comprise,” “have,” and “include” are open-ended linking verbs. Any forms or tenses of one or more of these verbs, such as “comprises,” “comprising,” “has,” “having,” “includes,” and “including,” are also open-ended. For example, any method that “comprises,” “has,” or “includes” one or more steps is not limited to possessing only those one or more steps and also covers other unlisted steps.

### Compositions and Methods for Transfection

The present disclosure provides compositions for altering the protein expression of a cell and which may result in differentiation of a cell. In some cases, a composition can be used to contact a cell. In some cases, a composition can be used to transfect a cell with a nucleic acid molecule. In some instances, a composition may comprise a saccharide. In some cases, a composition may comprise a nucleic acid molecule. In some cases, the composition may comprise a polymeric material. In some cases, a composition may comprise a saccharide, a nucleic acid molecule, a polymeric material, or any combination thereof. In some instances, the composition may comprise a saccharide and a nucleic acid molecule. In some instances, the composition may comprise a saccharide, a nucleic acid molecule, and a polymeric material. The compositions disclosed herein may further comprise a lipid, an anionic polymer, a nanoparticle, or a combination thereof. The saccharides provided herein may be modified. The saccharides provided herein may be functionalized or derivatized. The functionalization or derivatization may couple a chemical moiety described herein to the saccharide. The modified saccharide may have different properties relative to the unmodified saccharide. The modification of the saccharide may facilitate a nucleic acid molecule to be taken up by a cell. The modification may increase the net positive or cationic charge of a saccharide, relative to a unmodified saccharide.

The present disclosure also provides methods for using the compositions described herein. The method may facilitate a change in protein expression within a cell. In some cases, the method may comprise a method for differentiating a cell. In some cases, the method may comprise contacting a cell with a composition comprising a saccharide. In some cases, the method may comprise contacting a cell with a composition comprising a nucleic acid molecule. In some cases, the method may comprise encapsulating said cell with the polymeric material. In some cases, a method for differentiating a cell may comprise contacting a cell with a composition under a condition. In some cases, the condition may be sufficient to uptake said composition. In some cases, an uptake of the composition by a cell may facilitate a differentiation of a cell.

The compositions described herein may comprise any saccharides, nucleic acid molecules, lipids, nanoparticles, polyplex, or a combination thereof that is described herein. The methods described herein may use any compositions described herein.

### Saccharide

In some instances, a saccharide may comprise a carbohydrate. In some instances, a saccharide may comprise a carbohydrate moiety. A saccharide may be a sugar. A saccharide may comprise a sugar moiety. A saccharide may comprise a carbohydrate monomer or a monosaccharide. In some cases, a saccharide may comprise a polymeric form of monosaccharides. The monosaccharides of a polymeric form of a saccharide may be linked by glycosidic bonds. In some cases, a polymeric form of saccharide may comprise a disaccharide, an oligosaccharide, a polysaccharide, or a combination thereof. In some cases, a saccharide may comprise at least about 1, 2, 3, 4, 5, 6, 7, 8, 9, 10, 50, 100, 150, 200, 250, 300, 350, 400, 450, 500, 1000, 2000, 3000, 4000, 5000, 6000, 7000, 8000, 9000, 10000 or more carbohydrate moieties. In some cases, a saccharide may comprise at most about 1, 2, 3, 4, 5, 6, 7, 8, 9, 10, 50, 100, 150, 200, 250, 300, 350, 400, 450, 500, 1000, 2000, 3000, 4000, 5000, 6000, 7000, 8000, 9000, 10000, or 100000 carbohydrate moieties. In some cases, a saccharide may comprise at least about 1, 2, 3, 4, 5, 6, 7, 8, 9, 10, 50, 100, 150, 200, 250, 300, 350, 400, 450, 500, 1000, 2000, 3000, 4000, 5000, 6000, 7000, 8000, 9000, 10000 or more monosaccharides. In some cases, a saccharide may comprise at most about 1, 2, 3, 4, 5, 6, 7, 8, 9, 10, 50, 100, 150, 200, 250, 300, 350, 400, 450, 500, 1000, 2000, 3000, 4000, 5000, 6000, 7000, 8000, 9000, 10000, or 100000 monosaccharides.

In some instances, a saccharide may be functionalized or derivatized with various moieties. A saccharide or a derivative thereof may have cationic moieties. The term “derivative” or a grammatically equivalent term, when referring to a saccharide, refers to a modified saccharide modified via functionalization or derivatization of the saccharide with various chemical moieties. A corresponding cationic charge density of cationic moieties may be measured in mequivalent of basic group per gram of the saccharide molecule (mequiv/g). In some cases, the cationic charge density of cationic moieties may be determined by the polyelectrolyte titration method, conductometric titration, and/or acid/base titration. In some cases, the corresponding cationic charge density of cationic moieties of a saccharide or a derivative thereof may be at least about 0.5 mequiv/g, at least about 1 mequiv/g, at least about 1.5 mequiv/g, at least about 2 mequiv/g, at least about 2.5 mequiv/g, at least about 3 mequiv/g, at least about 3.5 mequiv/g, at least about 4 mequiv/g, at least about 4.5 mequiv/g, at least about 5 mequiv/g, at least about 5.5 mequiv/g, at least about 6 mequiv/g, at least about 6.5 mequiv/g, at least about 7 mequiv/g, at least about 7.5 mequiv/g, at least about 8 mequiv/g, at least about 8.5 mequiv/g, at least about 9 mequiv/g, at least about 9.5 mequiv/g, at least about 10 mequiv/g, at least about 10.5 mequiv/g, at least about 11 mequiv/g, at least about 11.5 mequiv/g, at least about 12 mequiv/g, at least about 12.5 mequiv/g, at least about 13 mequiv/g, at least about 13.5 mequiv/g, at least about 14 mequiv/g, at least about 14.5 mequiv/g, at least about 15 mequiv/g, at least about 15.5 mequiv/g, at least about 16 mequiv/g, at least about 16.5 mequiv/g, at least about 17 mequiv/g, at least about 17.5 mequiv/g, at least about 18 mequiv/g, at least about 18.5 mequiv/g, at least about 19 mequiv/g, at least about 19.5 mequiv/g, or at least about 20 mequiv/g. In some cases, the corresponding cationic charge density of cationic moieties of a saccharide or a derivative thereof may be at most about 0.5 mequiv/g, at most about 1 mequiv/g, at most about 1.5 mequiv/g, at most about 2 mequiv/g, at most about 2.5 mequiv/g, at most about 3 mequiv/g, at most about 3.5 mequiv/g, at most about 4 mequiv/g, at most about 4.5 mequiv/g, at most about 5 mequiv/g, at most about 5.5 mequiv/g, at most about 6 mequiv/g, at most about 6.5 mequiv/g, at most about 7 mequiv/g, at most about 7.5 mequiv/g, at most about 8 mequiv/g, at most about 8.5 mequiv/g, at most about 9 mequiv/g, at most about 9.5 mequiv/g, at most about 10 mequiv/g, at most about 10.5 mequiv/g, at most about 11 mequiv/g, at most about 11.5 mequiv/g, at most about 12 mequiv/g, at most about 12.5 mequiv/g, at most about 13 mequiv/g, at most about 13.5 mequiv/g, at most about 14 mequiv/g, at most about 14.5 mequiv/g, at most about 15 mequiv/g, at most about 15.5 mequiv/g, at most about 16 mequiv/g, at most about 16.5 mequiv/g, at most about 17 mequiv/g, at most about 17.5 mequiv/g, at most about 18 mequiv/g, at most about 18.5 mequiv/g, at most about 19 mequiv/g, at most about 19.5 mequiv/g, or at most about 20 mequiv/g. In some cases, the corresponding cationic charge density of cationic moieties of a saccharide or a derivative thereof may be from 0.005 to 2000 mequiv/g. In some cases, the corresponding cationic charge density of cationic moieties of a saccharide or a derivative thereof may be from 0.05 to 200 mequiv/g. In some cases, the corresponding cationic charge density of cationic moieties of a saccharide or a derivative thereof may be from 0.5 to 20 mequiv/g. In some cases, the corresponding cationic charge density of cationic moieties of a chitosan or derivative thereof may be at least about 0.5 mequiv/g, at least about 1 mequiv/g, at least about 1.5 mequiv/g, at least about 2 mequiv/g, at least about 2.5 mequiv/g, at least about 3 mequiv/g, at least about 3.5 mequiv/g, at least about 4 mequiv/g, at least about 4.5 mequiv/g, at least about 5 mequiv/g, at least about 5.5 mequiv/g, at least about 6 mequiv/g, at least about 6.5 mequiv/g, at least about 7 mequiv/g, at least about 7.5 mequiv/g, at least about 8 mequiv/g, at least about 8.5 mequiv/g, at least about 9 mequiv/g, at least about 9.5 mequiv/g, at least about 10 mequiv/g, at least about 10.5 mequiv/g, at least about 11 mequiv/g, at least about 11.5 mequiv/g, at least about 12 mequiv/g, at least about 12.5 mequiv/g, at least about 13 mequiv/g, at least about 13.5 mequiv/g, at least about 14 mequiv/g, at least about 14.5 mequiv/g, at least about 15 mequiv/g, at least about 15.5 mequiv/g, at least about 16 mequiv/g, at least about 16.5 mequiv/g, at least about 17 mequiv/g, at least about 17.5 mequiv/g, at least about 18 mequiv/g, at least about 18.5 mequiv/g, at least about 19 mequiv/g, at least about 19.5 mequiv/g, or at least about 20 mequiv/g. In some cases, the corresponding cationic charge density of cationic moieties of a chitosan or a derivative thereof may be at most about 0.5 mequiv/g, at most about 1 mequiv/g, at most about 1.5 mequiv/g, at most about 2 mequiv/g, at most about 2.5 mequiv/g, at most about 3 mequiv/g, at most about 3.5 mequiv/g, at most about 4 mequiv/g, at most about 4.5 mequiv/g, at most about 5 mequiv/g, at most about 5.5 mequiv/g, at most about 6 mequiv/g, at most about 6.5 mequiv/g, at most about 7 mequiv/g, at most about 7.5 mequiv/g, at most about 8 mequiv/g, at most about 8.5 mequiv/g, at most about 9 mequiv/g, at most about 9.5 mequiv/g, at most about 10 mequiv/g, at most about 10.5 mequiv/g, at most about 11 mequiv/g, at most about 11.5 mequiv/g, at most about 12 mequiv/g, at most about 12.5 mequiv/g, at most about 13 mequiv/g, at most about 13.5 mequiv/g, at most about 14 mequiv/g, at most about 14.5 mequiv/g, at most about 15 mequiv/g, at most about 15.5 mequiv/g, at most about 16 mequiv/g, at most about 16.5 mequiv/g, at most about 17 mequiv/g, at most about 17.5 mequiv/g, at most about 18 mequiv/g, at most about 18.5 mequiv/g, at most about 19 mequiv/g, at most about 19.5 mequiv/g, or at most about 20 mequiv/g. In some cases, the corresponding cationic charge density of cationic moieties of a chitosan or a derivative thereof may be from 0.005 to 2000 mequiv/g. In some cases, the corresponding cationic charge density of cationic moieties of a chitosan or a derivative thereof may be from 0.05 to 200 mequiv/g. In some cases, the corresponding cationic charge density of cationic moieties of a chitosan or a derivative thereof may be from 0.5 to 20 mequiv/g.

In some instances, a saccharide or a derivative thereof may comprise deacetylation of at least about 50%, at least about 55%, at least about 60%, at least about 65%, at least about 70%, at least about 75%, at least about 80%, at least about 85%, at least about 90%, or 100%, relative to a fully acetylated saccharide or a derivative thereof. In some instances, a saccharide or a derivative thereof may comprise deacetylation of at most about 95%, at most about 90%, at most about 85%, at most about 80%, at most about 75%, at most about 70%, at most about 65%, at most about 60%, at most about 55%, or at most about 50%, relative to a fully acetylated saccharide or a derivative thereof. The percentage of deacetylation may be measured by 1H NMR spectroscopy. The percentage of deacetylation may be measured by conductometric or potentiometric titration. The percentage of deacetylation of a saccharide (or other molecular entity described herein) may comprise a ratio of a saccharide (or other molecular entity described herein) that is fully acetylated relative to the saccharide (or other molecular entity described herein) in which the deacetylation is being measured. For example, the percentage of deacetylation of a saccharide may comprise a ratio of the number of acetylated groups that a saccharide contains when it is fully acetylated, relative to that of the saccharide, in which the deacetylation is being measured, contains.

In some instances, a saccharide or a derivative thereof may comprise an average molecular mass of at least about 20 kilodaltons (kDa). In some instances, a saccharide or a derivative thereof may comprise an average molecular mass of at least about 100 kDa. In some instances, a saccharide or a derivative thereof may comprise an average molecular mass of at least about 300 kDa. In some instances, a saccharide or a derivative thereof may comprise an average molecular mass of at least about 1000 kDa. In some instances, a saccharide or a derivative thereof may comprise an average molecular mass of at least about 3000 kDa. In some instances, a saccharide or a derivative thereof may comprise an average molecular mass of at least about 20 kDa, at least about 30 kDa, at least about 40 kDa, at least about 50 kDa, at least about 60 kDa, at least about 80 kDa, at least about 100 kDa, at least about 120 kDa, at least about 140 kDa, at least about 160 kDa, at least about 180 kDa, at least about 200 kDa, at least about 220 kDa, at least about 240 kDa, at least about 260 kDa, at least about 280 kDa, at least about 300 kDa, at least about 350 kDa, at least about 400 kDa, at least about 450 kDa, at least about 500 kDa, at least about 550 kDa, at least about 600 kDa, at least about 650 kDa, at least about 700 kDa, at least about 750 kDa, at least about 800 kDa, at least about 850 kDa, at least about 900 kDa, at least about 950 kDa, at least about 1000 kDa, at least about 1100 kDa, at least about 1200 kDa, at least about 1300 kDa, at least about 1400 kDa, at least about 1500 kDa, at least about 1600 kDa, at least about 1700 kDa, at least about 1800 kDa, at least about 1900 kDa, or at least about 2000 kDa. In some instances, a saccharide or a derivative thereof may comprise an average molecular mass of at most about 2000 kDa. In some instances, a saccharide or a derivative thereof may comprise an average molecular mass of at most about 1500 kDa. In some instances, a saccharide or a derivative thereof may comprise an average molecular mass of at most about 500 kDa. In some instances, a saccharide or a derivative thereof may comprise an average molecular mass of at most about 200 kDa. In some instances, a saccharide or a derivative thereof may comprise an average molecular mass of at most about 2000 kDa, at most about 1900 kDa, at most about 1800 kDa, at most about 1700 kDa, at most about 1600 kDa, at most about 1500 kDa, at most about 1400 kDa, at most about 1300 kDa, at most about 1200 kDa, at most about 1100 kDa, at most about 1000 kDa, at most about 900 kDa, at most about 800 kDa, at most about 700 kDa, at most about 600 kDa, at most about 500 kDa, at most about 400 kDa, at most about 300 kDa, at most about 200 kDa, at most about 100 kDa, at most about 80 kDa, at most about 60 kDa, at most about 40 kDa, or at most about 20 kDa. In some cases, a saccharide or a derivative thereof may comprise an average molecular mass from 0.2 kDa to 200000 kDa. In some cases, a saccharide or a derivative thereof may comprise an average molecular mass from 2 kDa to 20000 kDa. In some cases, a saccharide or a derivative thereof may comprise an average molecular mass from 20 kDa to 2000 kDa. In some instances, a saccharide or a derivative thereof may comprise an oligosaccharide or a polysaccharide.

A saccharide described herein may be present at a concentration at least about 1 picomolar (pM), 2 pM, 5 pM, 10 pM, 20 pM, 50 pM, 100 pM, 200 pM, 500 pM, 1 nanomolar (nM), 2 nM, 5 nM, 10 nM, 20 nM, 50 nM, 100 nM, 200 nM, 500 nM, 1 micromolar (μM), 2 μM, 5 μM, 10 μM, 20 μM, 50 μM, 100 μM, 200 μM, 500 μM, 1 millimolar (mM), 2 mM, 5 mM, 10 mM, 20 mM, 50 mM, 100 mM, 200 mM, 500 mM, 1 molar (M) or more within the composition. A saccharide described herein may be present at a concentration at most about 1 pM, 2 pM, 5 pM, 10 pM, 20 pM, 50 pM, 100 pM, 200 pM, 500 pM, 1 nM, 2 nM, 5 nM, 10 nM, 20 nM, 50 nM, 100 nM, 200 nM, 500 nM, 1 μM, 2 μM, 5 M, 10 μM, 20 μM, 50 μM, 100 μM, 200 μM, 500 μM, 1 mM, 2 mM, 5 mM, 10 mM, 20 mM, 50 mM, 100 mM, 200 mM, 500 mM, or 1 M within the composition.

In some instances, a reducing oligosaccharide modified saccharide may comprise a maltodextrin modified saccharide or a cellobiose modified saccharide. In some instances, a modified saccharide may comprise a mixture with cationic lipid, hydrophilized lipids, or a combination thereof. In some cases, a cationic lipid may be a hydrophilic lipid anchor, a linker group, a positively charged headgroup, or a combination thereof. In some cases, a cationic lipid may be a hydrophilic lipid anchor, a linker group, a positively charged headgroup, or a combination thereof. In some cases, a cationic lipid may be a hydrophilic lipid anchor, a linker group, a positively charged headgroup, or a combination thereof. The lipid anchor may comprise a fatty chain (e.g., derived from oleic or myristic acid) or a cholesterol group, which may determine the physical properties of the lipid bilayer (e.g., flexibility and the rate of lipid exchange).

Biodegradable lipids can be metabolized by various enzymes (e.g., esterase, peptidases) to minimize any toxicity. The linker can also provide sites for the introduction of novel side chains to enhance targeting, uptake, and trafficking. The positively charged head group on the cationic lipid may interact with the negatively charged nucleic acid (or a negatively charged moiety of a nucleic acid). The headgroups may be singly- or multiplicatively-charged as primary, secondary, tertiary, and/or quaternary amines. Multivalent headgroups, such as spermine, in a “T-shape” configuration may be efficient for promoting the uptake of nucleic acid molecules by a cell. In some case, an increase in the linker length may increase the uptake of nucleic acid molecules by a cell.

In some instances, a saccharide may comprise a modified saccharide. In some instances, a modified saccharide may comprise an aliphatic aldehyde modified saccharide. In some instances, an aliphatic aldehyde modified saccharide may comprise a hexanal aldehyde modified saccharide, a heptanal aldehyde modified saccharide, an octanal aldehyde modified saccharide, a nonanal aldehyde modified saccharide, or a decanal aldehyde modified saccharide. In some instances, an aliphatic aldehyde modified saccharide may comprise a decanal aldehyde modified saccharide. In some instances, an aliphatic aldehyde modified saccharide may comprise a hexanal aldehyde modified saccharide. In some instances, an aliphatic aldehyde modified saccharide may comprise a heptanal aldehyde modified saccharide. In some instances, an aliphatic aldehyde modified saccharide may comprise an octanal aldehyde modified saccharide. In some instances, an aliphatic aldehyde modified saccharide may comprise a nonanal aldehyde modified saccharide.

In some instances, a modified saccharide may comprise an aromatic aldehyde modified saccharide. In some instances, an aromatic aldehyde modified saccharide may comprise a benzaldehyde modified saccharide or a cinnamaldehyde modified saccharide. In some instances, an aromatic aldehyde modified saccharide may comprise a benzaldehyde modified saccharide. In some instances, an aromatic aldehyde modified saccharide may comprise a cinnamaldehyde modified saccharide.

In some instances, a modified saccharide may comprise a polyamine derivatized saccharide. In some instances, a polyamine derivatized saccharide may comprise a spermine modified saccharide, a spermidine modified saccharide, a putrescine modified saccharide, a diethylethylamine modified saccharide, a dimethylethylamine modified saccharide or an arginine modified saccharide. In some instances, a polyamine derivatized saccharide may comprise a spermine modified saccharide. In some instances, a polyamine derivatized saccharide may comprise a spermidine modified saccharide. In some instances, a polyamine derivatized saccharide may comprise a putrescine modified saccharide. In some instances, a polyamine derivatized saccharide may comprise a diethylethylamine modified saccharide. In some instances, a polyamine derivatized saccharide may comprise a dimethylethylamine modified saccharide. In some instances, a polyamine derivatized saccharide may comprise an arginine modified saccharide.

In some instances, a saccharide may comprise a lipid additive. In some cases, a saccharide may comprise an anion polymer additive. In some cases, a saccharide may comprise a lipid and/or anion polymer additive. In some instances, the lipid and/or anion polymer additive may comprise a phosphatidylcholine or a lecithin.

In some cases, a saccharide may comprise a chitosan, starch, amylose, amylopectin, dextran, dextrin, cellulose, hemicellulose, galactomannans, a hyaluronic acid, a pullulan, a heparin, alginates, carrageenan, xanthan gum, gellan gum, agarose, or a combination or derivative thereof. A derivative of a chemical compound may comprise a different chemical compound formed by a chemical reaction of the chemical compound. A derivate of a chemical may have a similar chemical, physical, or functional characteristic of the chemical. In some instances, a saccharide may comprise a chitosan or a derivative thereof. In some cases, a saccharide may also comprise a fructose, a galactose, a glucosamine, a glucose, a glyceraldehyde, a lactose, a maltose, a mannose, a ribose, a sucrose, a xylose, a cellulose, a pectin, a starch, a xanthan, a maltodextrin, a cellobiose, or a combination or derivative thereof.

In some instances, a chitosan may comprise a glucosamine. In some cases, a chitosan may comprise a polymer of pyranose monomers of glucosamine. In some cases, the chitosan may be linked by β-1,4 linkages of the pyranose monomers of glucosamine. In some cases, a chitosan may comprise at least about 10, 50, 100, 500, 1000, 1500, 2000, 2500, 3000, 3500, 4000, 4500, 5000, 5500, 6000, 6500, 7000, 7500, 8000, 8500, 9000, 9500, 10000 or more monomers of glucosamine. In some cases, a chitosan may comprise at most about 10, 50, 100, 500, 1000, 1500, 2000, 2500, 3000, 3500, 4000, 4500, 5000, 5500, 6000, 6500, 7000, 7500, 8000, 8500, 9000, 9500, 10000, or 100000 monomers of glucosamine. In some cases, a chitosan may comprise a chitin or derivative thereof. In some cases, a chitosan may comprise a poly-N-acetyl-D-glucosamine. In some cases, a chitosan may comprise poly-glucosamines. In some cases, a chitosan may comprise oligomers of glucosamines. A chitosan may be deacetylated. For example, a N-acetyl group of a chitosan may be removed through hydrolysis. In some cases, a hydrolytic enzyme may be used to modify a chitosan oligosaccharide. In other cases, a chitosanases may be to modify chitosan oligosaccharides. A chitosan may also be acetylated. In some cases, a chitosan may be binary hetero-polysaccharides composed of (1→4)-linked 2-acetamido-2-deoxy-β-D-glucose (GlcNAc, A-unit) and 2-amino-2-deoxy-β-D-glucose, (GlcN; D-unit).

In some cases, a chitosan may comprise a derivative of a chitosan. A derivative of a chitosan may comprise a nitrate, phosphate, sulphate, hydrochloride, glutamate, lactate or acetate salt of chitosan. In some cases, a chitosan may comprise an ester group. A chitosan may comprise an ether group. A chitosan may be formed by bonding of acyl and/or alkyl groups with OH groups. A chitosan may not be formed by bonding of acyl and/or alkyl groups the NH₂ groups. For example, a chitosan may comprise an O-alkyl ether of chitosan and O-acyl ester of chitosan.

In some instances, a chitosan may be isolated from a crustacean shell or from fungi or otherwise. In other cases, a chitosan may have a similar molecular structure of a chitosan isolated from a crustacean shell or fungi. In some cases, a chitosan may be antimicrobial. A chitosan may be an antibacterial, an antifungal, an antiparasitic, or any combination thereof.

In some instances, a chitosan may comprise a chitin. In some cases, a chitin may be isolated from a crustacean shell or fungi. In other cases, a chitin may have the same molecular structural of a chitin isolated from a crustacean shell or fungi. A chitin may comprise β-1,4-linked N-acetyl-glucosamine. A chitin may comprise a linear homopolymer of β-1,4-linked N-acetyl-glucosamine. In some cases, a chitin may be derived from alkaline deacetylation of a chitosan. In some cases, a chitin resulted from β-1,4-linked N-acetyl-glucosamine may comprise a polysaccharide composed of a glucosamine and/or a N-acetyl-glucosamine monomer. The glucosamine and N-acetyl-glucosamine monomers may be linked by β-1,4 glycosidic bonds.

In some instances, a saccharide may be cationic. In some cases, a saccharide may have a positive charge. In some cases, the amine group of a saccharide may be protonated in acidic conditions. In some instances, a saccharide may be a cation. In some cases, a saccharide may be a polycation. In some cases, a cationic saccharide is prepared by chemical modification to introduce primary, or secondary, or tertiary, or quaternary amine groups, or a combination thereof. In some instances, a cationic polysaccharide may be a chitosan. In some cases, a chitosan may have a positive charge. In some cases, the amine group of a chitosan may be protonated in acidic conditions. In some instances, a chitosan may be a cation. In some cases, a chitosan may be a polycation.

In some instances, a chitosan is configured to be cationic in an aqueous solution. In some instances, a chitosan is configured to be cationic in a neutral aqueous solution. In some instances, a saccharide is configured to be cationic in an aqueous solution. In some instances, a saccharide is configured to be cationic in a neutral aqueous solution.

In some instances, a chitosan or a derivative thereof may comprise deacetylation of at least about 50%, at least about 55%, at least about 60%, at least about 65%, at least about 70%, at least about 75%, at least about 80%, at least about 85%, at least about 90%, or 100%, relative to a fully acetylated chitosan or a derivative thereof. In some instances, a chitosan or a derivative thereof may comprise deacetylation of at most about 95%, at most about 90%, at most about 85%, at most about 80%, at most about 75%, at most about 70%, at most about 65%, at most about 60%, at most about 55%, or at most about 50%, relative to a fully acetylated chitosan or a derivative thereof.

In some instances, a chitosan or a derivative thereof may comprise an average molecular mass of at least about 200 kilodaltons (kDa). In some instances, a chitosan or a derivative thereof may comprise an average molecular mass of at least about 300 kDa. In some instances, a chitosan or a derivative thereof may comprise an average molecular mass of at least about 1000 kDa. In some instances, a chitosan or a derivative thereof may comprise an average molecular mass of at least about 3000 kDa. In some instances, a chitosan or a derivative thereof may comprise an average molecular mass of at least about 60 kDa, at least about 80 kDa, at least about 100 kDa, at least about 120 kDa, at least about 140 kDa, at least about 160 kDa, at least about 180 kDa, at least about 200 kDa, at least about 220 kDa, at least about 240 kDa, at least about 260 kDa, at least about 280 kDa, at least about 300 kDa, at least about 350 kDa, at least about 400 kDa, at least about 450 kDa, at least about 500 kDa, at least about 550 kDa, at least about 600 kDa, at least about 650 kDa, at least about 700 kDa, at least about 750 kDa, at least about 800 kDa, at least about 850 kDa, at least about 900 kDa, at least about 950 kDa, at least about 1000 kDa, at least about 1100 kDa, at least about 1200 kDa, at least about 1300 kDa, at least about 1400 kDa, at least about 1500 kDa, at least about 1600 kDa, at least about 1700 kDa, at least about 1800 kDa, at least about 1900 kDa, at least about 2000 kDa, at least about 2100 kDa, at least about 2200 kDa, at least about 2300 kDa, at least about 2400 kDa, at least about 2500 kDa, at least about 2600 kDa, at least about 2700 kDa, at least about 2800 kDa, at least about 2900 kDa, at least about 3000 kDa, at least about 3100 kDa, at least about 3200 kDa, at least about 3300 kDa, at least about 3400 kDa, or at least about 3500 kDa. In some instances, a chitosan or a derivative thereof may comprise an average molecular mass of at most about 2000 kDa. In some instances, a chitosan or a derivative thereof may comprise an average molecular mass of at most about 1500 kDa. In some instances, a chitosan or a derivative thereof may comprise an average molecular mass of at most about 500 kDa. In some instances, a chitosan or a derivative thereof may comprise an average molecular mass of at most about 200 kDa. In some instances, a chitosan or a derivative thereof may comprise an average molecular mass of at most about 3500 kDa, at most about 3400 kDa, at most about 3300 kDa, at most about 3200 kDa, at most about 3100 kDa, at most about 3000 kDa, at most about 2900 kDa, at most about 2800 kDa, at most about 2700 kDa, at most about 2600 kDa, at most about 2500 kDa, at most about 2400 kDa, at most about 2300 kDa, at most about 2200 kDa, at most about 2100 kDa, at most about 2000 kDa, at most about 1900 kDa, at most about 1800 kDa, at most about 1700 kDa, at most about 1600 kDa, at most about 1500 kDa, at most about 1400 kDa, at most about 1300 kDa, at most about 1200 kDa, at most about 1100 kDa, at most about 1000 kDa, at most about 900 kDa, at most about 800 kDa, at most about 700 kDa, at most about 600 kDa, at most about 500 kDa, at most about 400 kDa, at most about 300 kDa, at most about 200 kDa, or at most about 100 kDa. In some instances, a chitosan or a derivative thereof may comprise an oligomeric chitosan or a polymeric chitosan.

In some instances, a chitosan may comprise a modified chitosan. In some instances, a modified chitosan may comprise an aliphatic aldehyde modified chitosan. In some instances, an aliphatic aldehyde modified chitosan may comprise a hexanal aldehyde modified chitosan, a heptanal aldehyde modified chitosan, an octanal aldehyde modified chitosan, a nonanal aldehyde modified chitosan, or a decanal aldehyde modified chitosan. In some instances, an aliphatic aldehyde modified chitosan may comprise a decanal aldehyde modified chitosan. In some instances, an aliphatic aldehyde modified chitosan may comprise a hexanal aldehyde modified chitosan. In some instances, an aliphatic aldehyde modified chitosan may comprise a heptanal aldehyde modified chitosan. In some instances, an aliphatic aldehyde modified chitosan may comprise an octanal aldehyde modified chitosan. In some instances, an aliphatic aldehyde modified chitosan may comprise a nonanal aldehyde modified chitosan.

In some instances, a modified chitosan may comprise an aromatic aldehyde modified chitosan. In some instances, an aromatic aldehyde modified chitosan may comprise a benzaldehyde modified chitosan or a cinnamaldehyde modified chitosan. In some instances, an aromatic aldehyde modified chitosan may comprise a benzaldehyde modified chitosan. In some instances, an aromatic aldehyde modified chitosan may comprise a cinnamaldehyde modified chitosan.

In some instances, a modified chitosan may comprise a polyamine derivatized chitosan. In some instances, a polyamine derivatized chitosan may comprise a spermine modified chitosan, a spermidine modified chitosan, a putrescine modified chitosan, a diethylethylamine modified chitosan, a dimethylethylamine modified chitosan or an arginine modified chitosan. In some instances, a polyamine derivatized chitosan may comprise a spermine modified chitosan. In some instances, a polyamine derivatized chitosan may comprise a spermidine modified chitosan. In some instances, a polyamine derivatized chitosan may comprise a putrescine modified chitosan. In some instances, a polyamine derivatized chitosan may comprise a diethylethylamine modified chitosan. In some instances, a polyamine derivatized chitosan may comprise a dimethylethylamine modified chitosan. In some instances, a polyamine derivatized chitosan may comprise an arginine modified chitosan.

In some instances, a chitosan may comprise a lipid additive. In some cases, a chitosan may comprise an anionic polymer additive. In some cases, a chitosan may comprise a lipid and/or anion polymer additive. In some instances, the lipid and/or anion polymer additive a phosphatidylcholine or a lecithin.

In some instances, a modified chitosan may comprise a saccharide modified chitosan. In some instances, a saccharide modified chitosan may comprise a monosaccharide modified chitosan or a polysaccharide modified chitosan. In some instances, a monosaccharide modified chitosan may comprise a lactose modified chitosan, a mannose modified chitosan, a glucose modified chitosan, a galactose modified chitosan, a glucosamine modified chitosan, a sucrose modified chitosan, a maltose modified chitosan, a xylose modified chitosan, a ribose modified chitosan, a fructose modified chitosan, or a glyceraldehyde modified chitosan. In some instances, a monosaccharide modified chitosan may comprise a lactose modified chitosan. In some instances, a monosaccharide modified chitosan may comprise a mannose modified chitosan. In some instances, a monosaccharide modified chitosan may comprise a glucose modified chitosan. In some instances, a monosaccharide modified chitosan may comprise a galactose modified chitosan. In some instances, a monosaccharide modified chitosan may comprise a glucosamine modified chitosan. In some instances, a monosaccharide modified chitosan may comprise a sucrose modified chitosan. In some instances, a monosaccharide modified chitosan may comprise a maltose modified chitosan. In some instances, a monosaccharide modified chitosan may comprise a xylose modified chitosan. In some instances, a monosaccharide modified chitosan may comprise a ribose modified chitosan. In some instances, a monosaccharide modified chitosan may comprise a fructose modified chitosan. In some instances, a monosaccharide modified chitosan may comprise a glyceraldehyde modified chitosan . . .

In some instances, a polysaccharide modified chitosan may comprise an anionic polysaccharide modified chitosan or a reducing polysaccharide modified chitosan. In some instances, a polysaccharide modified chitosan may comprise an anionic polysaccharide modified chitosan. In some instances, a polysaccharide modified chitosan may comprise a reducing polysaccharide modified chitosan. In some instances, a modified saccharide may comprise a mixture with an anionic polysaccharide. In some instances, a modified saccharide may comprise a mixture with an anionic polysaccharide modified chitosan. In some instances, modified saccharide/anionic polysaccharide mixture may comprise a modified saccharide and an alginate, a carboxymethylated cellulose, a hyaluronic acid, a pectin, a carboxymethylated chitosan. In some instances, an anionic polysaccharide modified chitosan may comprise an alginate modified chitosan, a carboxymethylated cellulose modified chitosan, a hyaluronic acid modified chitosan, a pectin modified chitosan, a pullulan modified chitosan, a starch modified chitosan, or a xanthan gum modified chitosan. In some instances, an anionic polysaccharide modified chitosan may comprise an alginate modified chitosan. In some instances, an anionic polysaccharide modified chitosan may comprise a carboxymethylated cellulose modified chitosan. In some instances, an anionic polysaccharide modified chitosan may comprise a hyaluronic acid modified chitosan. In some instances, an anionic polysaccharide modified chitosan may comprise a pectin modified chitosan. In some instances, an anionic polysaccharide modified chitosan may comprise a pullulan modified chitosan. In some instances, an anionic polysaccharide modified chitosan may comprise a starch modified chitosan. In some instances, an anionic polysaccharide modified chitosan may comprise a xanthan gum modified chitosan. In some instances, a modified chitosan may comprise a phenol modified chitosan. A phenol may comprise a natural phenol. A natural phenol may comprise a chlorogenic acid, a ferulic acid, a caffeic acid, a gallic acid, or a combination thereof.

### Nucleic Acid

In some instances, a nucleic acid may be a nucleic acid molecule. In some cases, a nucleic acid may be a species/type of nucleic acids. A species or type of nucleic acid may share a common biological function, a mechanism of action, a structural characteristic, or a combination thereof. A species or type of nucleic acid may be a messenger ribonucleic acid (mRNA), a DNA, a micro ribonucleic acid (miRNA), a transfer ribonucleic acid (tRNA), a long non-coding RNA (lncRNA), a ribosomal ribonucleic acid (rRNA), a small nuclear RNA (snRNA), a piwi-interacting RNA (piRNA), a small nucleolar RNA (snoRNA), an extracellular RNA (exRNA), a small cajal body-specific RNA (scaRNA), a silencing ribonucleic acid (siRNA), self-amplifying RNA (saRNA), a YRNA (small noncoding RNA), a heterogeneous nuclear RNA (HnRNA), endless/circular RNA (eRNA), trans-amplifying RNA (ta-RNA), complementary DNA (cDNA), a transfer RNA (tRNA), a ribosomal RNA, a short-hairpin RNA (shRNA), or a ribozyme. A nucleic acid molecule may comprise one or more modified nucleotides, such as methylated nucleotides and nucleotide analogs. In some cases, a nucleic acid molecule may comprise a polymeric form of nucleotides. In some cases, a nucleic acid molecule may comprise a polynucleotide. In some cases, a nucleic acid molecule may comprise a modified polynucleotide. In some cases, a nucleic acid molecule may comprise a canonical or non-canonical nucleotide. A canonical nucleotide may comprise adenosine (A), cytosine (C), guanine (G), thymine (T), uracil (U), or variants thereof. In some cases, a nucleic acid may be single-stranded, double-stranded or triple stranded. In some cases, a nucleic acid may be single-stranded. In some cases, a nucleic acid may be double-stranded. In some cases, a nucleic acid may be single-stranded and double-stranded.

In some cases, a nucleic acid molecule may be linear or closed linear double-stranded (e.g., a doggybone DNA). In some cases, a nucleic acid molecule may be circular. In some cases, a nucleic acid molecule may be branched. A nucleic acid molecule may comprise a deoxyribonucleic acid (DNA) or ribonucleic acid (RNA). A nucleic acid molecule may comprise a peptide nucleic acid (PNA), an unlocked nucleic acid (UNA), a locked nucleic acid (LNA), an endless/circular RNA. LNA may comprise a structurally rigid modification (e.g., a 2′-O, 4′-C methylene bridge). UNA may comprise a flexible modification. The modification may restrict the flexibility of the ribofuranose ring and lock the structure into a rigid bicyclic formation. LNA may have increased thermal stability and hybridization specificity relative to an unmodified nucleic acid. UNA may have a structurally flexible modification (e.g., an acyclic analogue of RNA in which the bond between the C2′ and C3′ atoms of the ribose ring has been cleaved). UNA may lack the C2′—C4′ bond. PNA may comprise synthetic mimics of nucleic acid in which the deoxyribose phosphate backbone or ribose phosphate backbone is replaced by a pseudo-peptide polymer to which the nucleobases are linked. A nucleic acid molecule may comprise a coding sequence. A nucleic acid molecule may comprise a non-coding sequence. A nucleic acid molecule may comprise a coding or non-coding region of a gene or gene fragment, a locus defined from linkage analysis, an exons, an intron, an intein, or any combination thereof.

In some instances, a nucleic acid molecule may be monocistronic. In some cases, a nucleic acid molecule may be polycistronic. A monocistronic nucleic acid molecule may comprise one coding sequence, the coding sequence is configured to be recognized by a ribosome for the translation. A polycistronic nucleic acid molecule may comprise at least two coding sequences, each coding sequence is configured to be recognized by a ribosome for the translation of the coding sequence. In some cases, a nucleic acid molecule may comprise a sequence of a human gene. In some cases, a nucleic acid molecule may comprise a sequence of a non-human gene.

In some instances, a nucleic acid molecule may comprise a messenger ribonucleic acid (mRNA), a DNA, a micro ribonucleic acid (miRNA), a transfer ribonucleic acid (tRNA), a long non-coding RNA (lncRNA), a ribosomal ribonucleic acid (rRNA), a small nuclear RNA (snRNA), a piwi-interacting RNA (piRNA), a small nucleolar RNA (snoRNA), an extracellular RNA (exRNA), a small cajal body-specific RNA (scaRNA), a silencing ribonucleic acid (siRNA), self-amplifying RNA (saRNA), a YRNA (small noncoding RNA), a heterogeneous nuclear RNA (HnRNA), endless/circular RNA (eRNA), trans-amplifying RNA (ta-RNA), complementary DNA (cDNA) or a combination thereof. In some instances, a nucleic acid molecule may also comprise a transfer RNA (tRNA), a ribosomal RNA, a short-hairpin RNA (shRNA), a ribozyme, a recombinant nucleic acid, a branched nucleic acid, a plasmid, a vector, an isolated DNA, an isolated RNA, or any combination thereof. In some instances, a nucleic acid molecule may comprise an mRNA, a ta-RNA, a saRNA, a eRNA, or a combination thereof. A nucleic acid molecule may comprise a siRNA, a miRNA, a shRNA, a YRNA, or a combination thereof. A nucleic acid molecule may comprise a siRNA, a miRNA, or a combination thereof.

In some instances, a nucleic acid molecule may comprise an mRNA. The mRNA may be single-stranded. In some cases, the mRNA, when transfected into a cell, may allow a ribosome of the cell to translate the coding sequence of the mRNA and synthesize a protein based on the coding sequence of the mRNA.

In some instances, an mRNA may comprise a cell type-specific gene sequence. A cell type specific-gene sequence may comprise the sequence of a gene that is expressed or specifically expressed of the cell type. The cell type may comprise an ectoderm, an endoderm, or a mesoderm. The cell type may also comprise an adipogenic, angiogenic, cardiogenic, immunogenic, chondrogenic, endothelial, epithelial, hematopoietic, hepatogenic, myogenic, neurogenic, osteogenic, parenchymal, renal, retinal cell. In some cases, a cell may a T cell, a B cell, a natural killer cell, a neutrophil, an eosinophil, a basophil, a mast cell, a monocyte, a macrophage, or a dendritic cell. The cell type may also comprise any somatic cell, a stem cell or an immortalized cell. The stem cell or immortalized cell may comprise an induced pluripotent stem cell (iPSC), an embryonic stem cell (ESC), a mesenchymal stem cell (MSC), a satellite cell, a fibroblast.

In some instances, an mRNA may comprise an adipogenic, angiogenic, cardiogenic, immunogenic, chondrogenic, endothelial, epithelial, hematopoietic, hepatogenic, myogenic, neurogenic, osteogenic, parenchymal, renal, retinal gene sequence, or a combination thereof. In some cases, an mRNA may comprise an ectoderm, an endoderm, or a mesoderm gene sequence. In some cases, an mRNA may comprise an ectoderm gene sequence. In some cases, an mRNA may comprise an endoderm gene sequence. In some cases, an mRNA may comprise a mesoderm gene sequence.

In some cases, an mRNA may comprise a myogenic gene sequence. In other cases, an mRNA may comprise MYOD1. In some cases, an mRNA may comprise MYOG. In some cases, an mRNA may comprise MYF5. In some cases, an mRNA may comprise MYF6. In some cases, an mRNA may comprise PAX3. In some cases, an mRNA may comprise PAX7. In some cases, an mRNA may comprise MYOD1, MYOG, MYF5, MYF6, PAX3, PAX7, a fragment thereof, or a variant thereof. In some cases, an mRNA may comprise at least two of MYOD1, MYOG, MYF5, MYF6, PAX3, PAX7. In some cases, an mRNA may comprise at least three of MYOD1, MYOG, MYF5, MYF6, PAX3, PAX7. In some cases, an mRNA may comprise at least four of MYOD1, MYOG, MYF5, MYF6, PAX3, PAX7. In some cases, an mRNA may comprise at least five of MYOD1, MYOG, MYF5, MYF6, PAX3, PAX7. In some cases, an mRNA may comprise MYOD1, MYOG, MYF5, MYF6, PAX3, and PAX7.

In some cases, the MYOD1 mRNA may comprise at least about one nucleic acid modification in the 5′ untranslated region (UTR), 3′UTR and/or the length of the poly A-tail. In some cases, the at least about one modification has no impact on the ultimate amino acid sequence that is translated from the mRNA. In some cases, the MYOD1 mRNA is gene optimized. In some cases, the MYOD1 mRNA is codon optimized. Gene optimization may be carried out by any well-known methods in the art. Gene optimization may increase stability, reduce or inhibit degradation. Codon optimization may be carried out by any well-known methods in the art. Codon optimization may promote translation of the mRNA. In some cases, the MYOD1 mRNA is human MYOD1 mRNA. In some cases, the MYOD1 mRNA is non-human mammalian MYOD1 mRNA (e.g. cattle, buffalo, pigs, sheep, deer, etc.), bird MYOD1 mRNA (e.g. chicken, ducks, ostrich, turkey, pheasant, etc.), fish MYOD1 mRNA (e.g. swordfish, salmon, tuna, sea bass, trout, catfish, etc.), invertebrate MYOD1 mRNA (e.g. lobster, crab, shrimp, clams, oysters, mussels, sea urchin, etc.), reptile MYOD1 mRNA (e.g. snake, alligator, turtle, etc.), or amphibian MYOD1 mRNA (e.g. frogs). In some cases the MYOD1 mRNA is porcine MYOD1 mRNA.

In some cases, an mRNA may comprise an adipogenic gene sequence. In some cases, an mRNA may comprise PPARY, adiponectin, FATP1-6, FABP4, GLUT4, Leptin, AdipoR1-2, CD137, or any combination thereof. In some cases, an mRNA encodes PPARY. In some cases, an mRNA encodes adiponectin. In some cases, an mRNA encodes FATP1-6. In some cases, an mRNA encodes FABP4. In some cases, an mRNA encodes GLUT4. In some cases, an mRNA encodes Leptin. In some cases, an mRNA may comprise AdipoR1-2. In some cases, an mRNA encodes CD137. In some cases, an mRNA may comprise at least two of PPARY, adiponectin, FATP1-6, FABP4, GLUT4, Leptin, AdipoR1-2, CD137. In some cases, an mRNA may comprise at least three of PPARY, adiponectin, FATP1-6, FABP4, GLUT4, Leptin, AdipoR1-2, CD137. In some cases, an mRNA may comprise at least four of PPARY, adiponectin, FATP1-6, FABP4, GLUT4, Leptin, AdipoR1-2, CD137. In some cases, an mRNA may comprise at least five of PPARY, adiponectin, FATP1-6, FABP4, GLUT4, Leptin, AdipoR1-2, CD137. In some cases, an mRNA may comprise at least six of PPARY, adiponectin, FATP1-6, FABP4, GLUT4, Leptin, AdipoR1-2, CD137. In some cases, an mRNA may comprise at least seven of PPARY, adiponectin, FATP1-6, FABP4, GLUT4, Leptin, AdipoR1-2, CD137. In some cases, an mRNA may comprise at PPARY, adiponectin, FATP1-6, FABP4, GLUT4, Leptin, AdipoR1-2, and CD137.

In some cases, an mRNA may comprise an angiogenic gene sequence. In some cases, an mRNA may comprise a cardiogenic gene sequence. In some cases, an mRNA may comprise a chondrogenic gene sequence. In some cases, an mRNA may comprise an endothelial gene sequence. In some cases, an mRNA may comprise an epithelial gene sequence. In some cases, an mRNA may comprise a hematopoietic gene sequence. In some cases, an mRNA may comprise a hepatogenic gene sequence. In some cases, an mRNA may comprise a neurogenic gene sequence. In some cases, an mRNA may comprise an osteogenic gene sequence. In some cases, an mRNA may comprise a parenchymal gene sequence. In some cases, an mRNA may comprise a renal gene sequence. In some cases, an mRNA may comprise a retinal gene sequence.

In some instances, an mRNA may be monocistronic. In some cases, an mRNA may be polycistronic. A monocistronic mRNA may comprise one coding sequence, the coding sequence is configured to be recognized by a ribosome for the translation. A polycistronic mRNA may comprise at least two coding sequences, each coding sequence is configured to be recognized by a ribosome for the translation of the coding sequence. In some instances, an mRNA may comprise an RNA-regulatory element. In some cases, an mRNA comprising an RNA-regulatory element may reduce or inhibit degradation of the mRNA at least about 5%, at least about 10%, at least about 15%, at least about 20%, at least about 25%, at least about 30%, at least about 35%, at least about 40%, at least about 45%, at least about 50%, at least about 55%, at least about 60%, at least about 65%, at least about 70%, at least about 75%, at least about 80%, at least about 85%, at least about 90%, or at least about 95%, relative to an mRNA without the RNA-regulatory element.

In some cases, an RNA-regulatory element may comprise a transcriptional regulatory element, a co-transcriptional regulatory element, a post-transcriptional regulatory element, a translational regulatory element, or any combination thereof. In some cases, an RNA-regulatory element may comprise a 5′-cap, 5′ UTRs, a 3′-UTR, a poly-A tail modification, or any combination thereof. In some cases, an RNA-regulatory element may comprise a 5′-cap. In some cases, an RNA-regulatory element may comprise a 5′ UTR. In some cases, an RNA-regulatory element may comprise a 3′-UTR. In some cases, an RNA-regulatory element may comprise a poly-A tail modification. In some cases, an RNA-regulatory element may comprise at least two of a 5′-cap, 5′ UTRs, 3′-UTRs, or a poly-A tail modification. In some cases, an RNA-regulatory element may comprise a 5′-cap, 5′ UTRs, a 3′-UTR, and a poly-A tail modification. Other RNA-regulatory elements may comprise an intron sequence, a stop codon sequence, a translation start site, an RNA-localization sequence, or any combination thereof.

In some instances, a nucleic acid molecule may comprise a cDNA. In some instances, a cDNA may comprise an adipogenic, angiogenic, cardiogenic, chondrogenic, endothelial, epithelial, hematopoietic, hepatogenic, myogenic, neurogenic, osteogenic, parenchymal, renal, retinal gene sequence, or a combination thereof. In some cases, a cDNA may comprise an ectoderm, an endoderm, or a mesoderm gene sequence. In some cases, a cDNA may comprise an ectoderm gene sequence. In some cases, a cDNA may comprise an endoderm gene sequence. In some cases, a cDNA may comprise a mesoderm gene sequence.

In some cases, a cDNA may comprise a myogenic gene sequence. In other cases, a cDNA may comprise MYOD1. In some cases, a cDNA may comprise MYOG. In some cases, a cDNA may comprise MYF5. In some cases, a cDNA may comprise MYF6. In some cases, a cDNA may comprise PAX3. In some cases, a cDNA may comprise PAX7. In some cases, a cDNA may comprise MYOD1, MYOG, MYF5, MYF6, PAX3, PAX7, a fragment thereof, or a variant thereof. In some cases, a cDNA may comprise at least two of MYOD1, MYOG, MYF5, MYF6, PAX3, PAX7. In some cases, a cDNA may comprise at least three of MYOD1, MYOG, MYF5, MYF6, PAX3, PAX7. In some cases, a cDNA may comprise at least four of MYOD1, MYOG, MYF5, MYF6, PAX3, PAX7. In some cases, a cDNA may comprise at least five of MYOD1, MYOG, MYF5, MYF6, PAX3, PAX7. In some cases, a cDNA may comprise MYOD1, MYOG, MYF5, MYF6, PAX3, and PAX7.

In some cases, a cDNA may comprise an adipogenic gene sequence. In some cases, a cDNA may comprise PPARY, adiponectin, FATP1-6, FABP4, GLUT4, Leptin, AdipoR1-2, CD137, or any combination thereof. In some cases, a cDNA encodes PPARY. In some cases, a cDNA encodes adiponectin. In some cases, a cDNA encodes FATP1-6. In some cases, a cDNA encodes FABP4. In some cases, a cDNA encodes GLUT4. In some cases, a cDNA encodes Leptin. In some cases, a cDNA may comprise AdipoR1-2. In some cases, a cDNA encodes CD137. In some cases, a cDNA may comprise at least two of PPARY, adiponectin, FATP1-6, FABP4, GLUT4, Leptin, AdipoR1-2, CD137. In some cases, a cDNA may comprise at least three of PPARY, adiponectin, FATP1-6, FABP4, GLUT4, Leptin, AdipoR1-2, CD137. In some cases, a cDNA may comprise at least four of PPARY, adiponectin, FATP1-6, FABP4, GLUT4, Leptin, AdipoR1-2, CD137. In some cases, a cDNA may comprise at least five of PPARY, adiponectin, FATP1-6, FABP4, GLUT4, Leptin, AdipoR1-2, CD137. In some cases, a cDNA may comprise at least six of PPARY, adiponectin, FATP1-6, FABP4, GLUT4, Leptin, AdipoR1-2, CD137. In some cases, a cDNA may comprise at least seven of PPARY, adiponectin, FATP1-6, FABP4, GLUT4, Leptin, AdipoR1-2, CD137. In some cases, a cDNA may comprise at PPARY, adiponectin, FATP1-6, FABP4, GLUT4, Leptin, AdipoR1-2, and CD137.

In some cases, a cDNA may comprise an angiogenic gene sequence. In some cases, a cDNA may comprise a cardiogenic gene sequence. In some cases, a cDNA may comprise a chondrogenic gene sequence. In some cases, a cDNA may comprise an endothelial gene sequence. In some cases, a cDNA may comprise an epithelial gene sequence. In some cases, a cDNA may comprise hematopoietic gene sequence. In some cases, a cDNA may comprise hepatogenic gene sequence. In some cases, a cDNA may comprise a neurogenic gene sequence. In some cases, a cDNA may comprise an osteogenic gene sequence. In some cases, a cDNA may comprise a parenchymal gene sequence. In some cases, a cDNA may comprise a renal gene sequence. In some cases, a cDNA may comprise a retinal gene sequence.

In some instances, a nucleic acid molecule may comprise a saRNA. In some instances, a saRNA may comprise an adipogenic, angiogenic, cardiogenic, chondrogenic, endothelial, epithelial, hematopoietic, hepatogenic, myogenic, neurogenic, osteogenic, parenchymal, renal, retinal gene sequence, or a combination thereof. In some cases, a saRNA may comprise an ectoderm, an endoderm, or a mesoderm gene sequence. In some cases, a saRNA may comprise an ectoderm gene sequence. In some cases, a saRNA may comprise an endoderm gene sequence. In some cases, a saRNA may comprise a mesoderm gene sequence.

In some cases, a saRNA may comprise a myogenic gene sequence. In other cases, a saRNA may comprise MYOD1. In some cases, a saRNA may comprise MYOG. In some cases, a saRNA may comprise MYF5. In some cases, a saRNA may comprise MYF6. In some cases, a saRNA may comprise PAX3. In some cases, a saRNA may comprise PAX7. In some cases, a saRNA may comprise MYOD1, MYOG, MYF5, MYF6, PAX3, PAX7, a fragment thereof, or a variant thereof. In some cases, a saRNA may comprise at least two of MYOD1, MYOG, MYF5, MYF6, PAX3, PAX7. In some cases, a saRNA may comprise at least three of MYOD1, MYOG, MYF5, MYF6, PAX3, PAX7. In some cases, a saRNA may comprise at least four of MYOD1, MYOG, MYF5, MYF6, PAX3, PAX7. In some cases, a saRNA may comprise at least five of MYOD1, MYOG, MYF5, MYF6, PAX3, PAX7. In some cases, a saRNA may comprise MYOD1, MYOG, MYF5, MYF6, PAX3, and PAX7.

In some cases, a saRNA may comprise an adipogenic gene sequence. In some cases, a saRNA may comprise PPARY, adiponectin, FATP1-6, FABP4, GLUT4, Leptin, AdipoR1-2, CD137, or any combination thereof. In some cases, a saRNA encodes PPARY. In some cases, a saRNA encodes adiponectin. In some cases, a saRNA encodes FATP1-6. In some cases, a saRNA encodes FABP4. In some cases, a saRNA encodes GLUT4. In some cases, a saRNA encodes Leptin. In some cases, a saRNA may comprise AdipoR 1-2. In some cases, a saRNA encodes CD137. In some cases, a saRNA may comprise at least two of PPARY, adiponectin, FATP1-6, FABP4, GLUT4, Leptin, AdipoR1-2, CD137. In some cases, a saRNA may comprise at least three of PPARY, adiponectin, FATP1-6, FABP4, GLUT4, Leptin, AdipoR1-2, CD137. In some cases, a saRNA may comprise at least four of PPARY, adiponectin, FATP1-6, FABP4, GLUT4, Leptin, AdipoR1-2, CD137. In some cases, a saRNA may comprise at least five of PPARY, adiponectin, FATP1-6, FABP4, GLUT4, Leptin, AdipoR1-2, CD137. In some cases, a saRNA may comprise at least six of PPARY, adiponectin, FATP1-6, FABP4, GLUT4, Leptin, AdipoR1-2, CD137. In some cases, a saRNA may comprise at least seven of PPARY, adiponectin, FATP1-6, FABP4, GLUT4, Leptin, AdipoR1-2, CD137. In some cases, a saRNA may comprise at PPARY, adiponectin, FATP1-6, FABP4, GLUT4, Leptin, AdipoR1-2, and CD137.

In some cases, a saRNA may comprise an angiogenic gene sequence. In some cases, a saRNA may comprise a cardiogenic gene sequence. In some cases, a saRNA may comprise a chondrogenic gene sequence. In some cases, a saRNA may comprise an endothelial gene sequence. In some cases, a saRNA may comprise an epithelial gene sequence. In some cases, a saRNA may comprise hematopoietic gene sequence. In some cases, a saRNA may comprise hepatogenic gene sequence. In some cases, a saRNA may comprise a neurogenic gene sequence. In some cases, a saRNA may comprise an osteogenic gene sequence. In some cases, a saRNA may comprise a parenchymal gene sequence. In some cases, a saRNA may comprise a renal gene sequence. In some cases, a saRNA may comprise a retinal gene sequence.

In some instances, a nucleic acid molecule may comprise a eRNA. In some instances, a eRNA may comprise an adipogenic, angiogenic, cardiogenic, chondrogenic, endothelial, epithelial, hematopoietic, hepatogenic, myogenic, neurogenic, osteogenic, parenchymal, renal, retinal gene sequence, or a combination thereof. In some cases, a eRNA may comprise an ectoderm, an endoderm, or a mesoderm gene sequence. In some cases, a eRNA may comprise an ectoderm gene sequence. In some cases, a eRNA may comprise an endoderm gene sequence. In some cases, a eRNA may comprise a mesoderm gene sequence.

In some cases, a eRNA may comprise a myogenic gene sequence. In other cases, a eRNA may comprise MYOD1. In some cases, a eRNA may comprise MYOG. In some cases, a eRNA may comprise MYF5. In some cases, a eRNA may comprise MYF6. In some cases, a eRNA may comprise PAX3. In some cases, a eRNA may comprise PAX7. In some cases, a eRNA may comprise MYOD1, MYOG, MYF5, MYF6, PAX3, PAX7, a fragment thereof, or a variant thereof. In some cases, a eRNA may comprise at least two of MYOD1, MYOG, MYF5, MYF6, PAX3, PAX7. In some cases, a eRNA may comprise at least three of MYOD1, MYOG, MYF5, MYF6, PAX3, PAX7. In some cases, a eRNA may comprise at least four of MYOD1, MYOG, MYF5, MYF6, PAX3, PAX7. In some cases, a eRNA may comprise at least five of MYOD1, MYOG, MYF5, MYF6, PAX3, PAX7. In some cases, a eRNA may comprise MYOD1, MYOG, MYF5, MYF6, PAX3, and PAX7.

In some cases, a eRNA may comprise an adipogenic gene sequence. In some cases, a eRNA may comprise PPARY, adiponectin, FATP1-6, FABP4, GLUT4, Leptin, AdipoR1-2, CD137, or any combination thereof. In some cases, a eRNA encodes PPARY. In some cases, a eRNA encodes adiponectin. In some cases, a eRNA encodes FATP1-6. In some cases, a eRNA encodes FABP4. In some cases, a eRNA encodes GLUT4. In some cases, a eRNA encodes Leptin. In some cases, a eRNA may comprise AdipoR1-2. In some cases, a eRNA encodes CD137. In some cases, a eRNA may comprise at least two of PPARY, adiponectin, FATP1-6, FABP4, GLUT4, Leptin, AdipoR 1-2, CD137. In some cases, a eRNA may comprise at least three of PPARY, adiponectin, FATP1-6, FABP4, GLUT4, Leptin, AdipoR1-2, CD137. In some cases, a eRNA may comprise at least four of PPARY, adiponectin, FATP1-6, FABP4, GLUT4, Leptin, AdipoR1-2, CD137. In some cases, a eRNA may comprise at least five of PPARY, adiponectin, FATP1-6, FABP4, GLUT4, Leptin, AdipoR1-2, CD137. In some cases, a eRNA may comprise at least six of PPARY, adiponectin, FATP1-6, FABP4, GLUT4, Leptin, AdipoR1-2, CD137. In some cases, a eRNA may comprise at least seven of PPARY, adiponectin, FATP1-6, FABP4, GLUT4, Leptin, AdipoR1-2, CD137. In some cases, a eRNA may comprise at PPARY, adiponectin, FATP1-6, FABP4, GLUT4, Leptin, AdipoR1-2, and CD137.

In some cases, a eRNA may comprise an angiogenic gene sequence. In some cases, a eRNA may comprise a cardiogenic gene sequence. In some cases, a eRNA may comprise a chondrogenic gene sequence. In some cases, a eRNA may comprise an endothelial gene sequence. In some cases, a eRNA may comprise an epithelial gene sequence. In some cases, a eRNA may comprise hematopoietic gene sequence. In some cases, a eRNA may comprise hepatogenic gene sequence. In some cases, a eRNA may comprise a neurogenic gene sequence. In some cases, a eRNA may comprise an osteogenic gene sequence. In some cases, a eRNA may comprise a parenchymal gene sequence. In some cases, a eRNA may comprise a renal gene sequence. In some cases, a eRNA may comprise a retinal gene sequence.

In some instances, a nucleic acid molecule may comprise a ta-RNA. In some instances, a ta-RNA may comprise an adipogenic, angiogenic, cardiogenic, chondrogenic, endothelial, epithelial, hematopoietic, hepatogenic, myogenic, neurogenic, osteogenic, parenchymal, renal, retinal gene sequence, or a combination thereof. In some cases, a ta-RNA may comprise an ectoderm, an endoderm, or a mesoderm gene sequence. In some cases, a ta-RNA may comprise an ectoderm gene sequence. In some cases, a ta-RNA may comprise an endoderm gene sequence. In some cases, a ta-RNA may comprise a mesoderm gene sequence.

In some cases, a ta-RNA may comprise a myogenic gene sequence. In other cases, a ta-RNA may comprise MYOD1. In some cases, a ta-RNA may comprise MYOG. In some cases, a ta-RNA may comprise MYF5. In some cases, a ta-RNA may comprise MYF6. In some cases, a ta-RNA may comprise PAX3. In some cases, a ta-RNA may comprise PAX7. In some cases, a ta-RNA may comprise MYOD1, MYOG, MYF5, MYF6, PAX3, PAX7, a fragment thereof, or a variant thereof. In some cases, a ta-RNA may comprise at least two of MYOD1, MYOG, MYF5, MYF6, PAX3, PAX7. In some cases, a ta-RNA may comprise at least three of MYOD1, MYOG, MYF5, MYF6, PAX3, PAX7. In some cases, a ta-RNA may comprise at least four of MYOD1, MYOG, MYF5, MYF6, PAX3, PAX7. In some cases, a ta-RNA may comprise at least five of MYOD1, MYOG, MYF5, MYF6, PAX3, PAX7. In some cases, a ta-RNA may comprise MYOD1, MYOG, MYF5, MYF6, PAX3, and PAX7.

In some cases, a ta-RNA may comprise an adipogenic gene sequence. In some cases, a ta-RNA may comprise PPARY, adiponectin, FATP1-6, FABP4, GLUT4, Leptin, AdipoR1-2, CD137, or any combination thereof. In some cases, a ta-RNA encodes PPARY. In some cases, a ta-RNA encodes adiponectin. In some cases, a ta-RNA encodes FATP1-6. In some cases, a ta-RNA encodes FABP4. In some cases, a ta-RNA encodes GLUT4. In some cases, a ta-RNA encodes Leptin. In some cases, a ta-RNA may comprise AdipoR1-2. In some cases, a ta-RNA encodes CD137. In some cases, a ta-RNA may comprise at least two of PPARY, adiponectin, FATP1-6, FABP4, GLUT4, Leptin, AdipoR1-2, CD137. In some cases, a ta-RNA may comprise at least three of PPARY, adiponectin, FATP1-6, FABP4, GLUT4, Leptin, AdipoR1-2, CD137. In some cases, a ta-RNA may comprise at least four of PPARY, adiponectin, FATP1-6, FABP4, GLUT4, Leptin, AdipoR1-2, CD137. In some cases, a ta-RNA may comprise at least five of PPARY, adiponectin, FATP1-6, FABP4, GLUT4, Leptin, AdipoR1-2, CD137. In some cases, a ta-RNA may comprise at least six of PPARY, adiponectin, FATP1-6, FABP4, GLUT4, Leptin, AdipoR1-2, CD137. In some cases, a ta-RNA may comprise at least seven of PPARY, adiponectin, FATP1-6, FABP4, GLUT4, Leptin, AdipoR1-2, CD137. In some cases, a ta-RNA may comprise at PPARY, adiponectin, FATP1-6, FABP4, GLUT4, Leptin, AdipoR1-2, and CD137.

In some cases, a ta-RNA may comprise an angiogenic gene sequence. In some cases, a ta-RNA may comprise a cardiogenic gene sequence. In some cases, a ta-RNA may comprise a chondrogenic gene sequence. In some cases, a ta-RNA may comprise an endothelial gene sequence. In some cases, a ta-RNA may comprise an epithelial gene sequence. In some cases, a ta-RNA may comprise hematopoietic gene sequence. In some cases, a ta-RNA may comprise hepatogenic gene sequence. In some cases, a ta-RNA may comprise a neurogenic gene sequence. In some cases, a ta-RNA may comprise an osteogenic gene sequence. In some cases, a ta-RNA may comprise a parenchymal gene sequence. In some cases, a ta-RNA may comprise a renal gene sequence. In some cases, a ta-RNA may comprise a retinal gene sequence.

In some instances, a nucleic acid molecule may comprise a miRNA or a siRNA. In some instances, a nucleic acid molecule may comprise a miRNA. In some instances, a nucleic acid molecule may comprise a siRNA. In some cases, a miRNA or a siRNA may comprise a polynucleotide sequence that facilitates a reduction of pluripotency of a cell. In some cases, a miRNA or a siRNA may comprise a polynucleotide sequence that facilitates differentiation by reducing pluripotency of a cell. In some cases, a polynucleotide sequence may comprise a polynucleotide sequence that facilitates knockdown/reduction of a pluripotency of a cell by POUF51 (OCT3/4), KLF4, or SOX2, or a complementary sequence thereof.

In some cases, siRNA may be a class of short, double-stranded RNA non-coding RNA molecules which may interfere with the expression of specific genes with complementary nucleotide sequences. In some cases, siRNA may interfere with gene expression by degrading mRNA after transcription or preventing translation. In some cases, siRNAs may be 10-30 base pairs in length with phosphorylated 5′ ends and hydroxylated 3′ ends. In some cases, siRNAs may target complementary mRNA for degradation, thus preventing translation. In some cases,

In some cases, a siRNA may comprise a sequence or complementary sequence of POU5F1 (OCT3/4), SOX2, nanog, SSEA-4, KLF4 and/or TRA-1-60. In some cases, a siRNA may comprise a sequence or complementary sequence of POU5F1 (OCT3/4). In some cases, a siRNA may comprise a sequence or complementary sequence of SOX2. In some cases, a siRNA may comprise a sequence or complementary sequence of nanog. In some cases, a siRNA may comprise a sequence or complementary sequence of SSEA-4. In some cases, a siRNA may comprise a sequence or complementary sequence of KLF4. In some cases, a siRNA may comprise a sequence or complementary sequence of TRA-1-60. In some cases, a siRNA may comprises at least two sequences or complementary sequences of POU5F1 (OCT3/4), SOX2, nanog, SSEA-4, KLF4 or TRA-1-60. In some cases, a siRNA may comprises at least three sequences or complementary sequences of POU5F1 (OCT3/4), SOX2, nanog, SSEA-4, KLF4 or TRA-1-60. In some cases, a siRNA may comprises at least four sequences or complementary sequences of POU5F1 (OCT3/4), SOX2, nanog, SSEA-4, KLF4 or TRA-1-60. In some cases, a siRNA may comprises sequences or complementary sequences of POU5F1 (OCT3/4), SOX2, nanog, SSEA-4, KLF4 and TRA-1-60.

A shRNA, in some cases, may be converted to a siRNA by a cell. In some cases, a nucleic acid molecule may comprise a shRNA. In some cases, a shRNA may comprise a sequence or complementary sequence of POU5F1 (OCT3/4), SOX2, nanog, SSEA-4, KLF4 and/or TRA-1-60. In some cases, a shRNA may comprise a sequence or complementary sequence of POU5F1 (OCT3/4). In some cases, a shRNA may comprise a sequence or complementary sequence of SOX2. In some cases, a shRNA may comprise a sequence or complementary sequence of nanog. In some cases, a shRNA may comprise a sequence or complementary sequence of SSEA-4. In some cases, a shRNA may comprise a sequence or complementary sequence of KLF4. In some cases, a shRNA may comprise a sequence or complementary sequence of TRA-1-60. In some cases, a shRNA may comprises at least two sequences or complementary sequences of POU5F1 (OCT3/4), SOX2, nanog, SSEA-4, KLF4 or TRA-1-60. In some cases, a shRNA may comprises at least three sequences or complementary sequences of POU5F1 (OCT3/4), SOX2, nanog, SSEA-4, KLF4 or TRA-1-60. In some cases, a shRNA may comprises at least four sequences or complementary sequences of POU5F1 (OCT3/4), SOX2, nanog, SSEA-4, KLF4 or TRA-1-60. In some cases, a shRNA may comprises sequences or complementary sequences of POU5F1 (OCT3/4), SOX2, nanog, SSEA-4, KLF4 and TRA-1-60.

A miRNA can be a small non-coding RNA molecule that functions in RNA silencing and post-transcriptional regulation of gene expression. In some cases, miRNAs base-pair with complementary sequences within mRNA molecules, silencing the mRNA molecules. In some cases, silencing may be achieved upon binding of the miRNA to the 3′UTR of the target mRNA through cleavage of the mRNA strand into two pieces, destabilization of mRNA through shortening the poly-A tail, or through inefficient translation of the mRNA into proteins by ribosomes. In some cases, modulation of myogenic gene expression may occur through miRNAs. miRNAs that may modulate myogenic gene expression may comprise miR-1, miR-24, miR-26a, miR-27b, miR-29b/c, miR-125b, miR-133, miR-181, miR-206, miR-208b/499, miR-214, miR-221/222, miR-322/424, mi486, or miR-503. In some cases, a miRNA may be expressed in a cell type. In some cases, a miRNA may be specifically expressed in a cell type. miRNAs may be specifically expressed in cardiac and skeletal muscles under the control of the myogenic transcription factors SRF, MyoD or MEF2 where they may regulate processes of skeletal myogenesis such as myoblast/satellite cell proliferation and differentiation.

In some cases, a miRNA may comprise a sequence or complementary sequence of POU5F1 (OCT3/4), SOX2, nanog, SSEA-4, KLF4 and/or TRA-1-60. In some cases, a miRNA may comprise a sequence or complementary sequence of POU5F1 (OCT3/4). In some cases, a miRNA may comprise a sequence or complementary sequence of SOX2. In some cases, a miRNA may comprise a sequence or complementary sequence of nanog. In some cases, a miRNA may comprise a sequence or complementary sequence of SSEA-4. In some cases, a miRNA may comprise a sequence or complementary sequence of KLF4. In some cases, a miRNA may comprise a sequence or complementary sequence of TRA-1-60. In some cases, a miRNA may comprises at least two sequences or complementary sequences of POU5F1 (OCT3/4), SOX2, nanog, SSEA-4, KLF4 or TRA-1-60. In some cases, a miRNA may comprises at least three sequences or complementary sequences of POU5F1 (OCT3/4), SOX2, nanog, SSEA-4, KLF4 or TRA-1-60. In some cases, a miRNA may comprises at least four sequences or complementary sequences of POU5F1 (OCT3/4), SOX2, nanog, SSEA-4, KLF4 or TRA-1-60. In some cases, a miRNA may comprises sequences or complementary sequences of POU5F1 (OCT3/4), SOX2, nanog, SSEA-4, KLF4 and TRA-1-60.

In some cases, a nucleic acid molecule may comprise a saRNA. In some cases, a saRNA may comprise a sequence or complementary sequence of Mrf4, Pax7, PAX3, MYOG, MYF5, or MYF6, or a combination thereof. In some cases, a saRNA may comprise a sequence or complementary sequence of Mrf4. In some cases, a saRNA may comprise a sequence or complementary sequence of Pax7. In some cases, a saRNA may comprise a sequence or complementary sequence of PAX3. In some cases, a saRNA may comprise a sequence or complementary sequence of MYOG. In some cases, a saRNA may comprise a sequence or complementary sequence of MYF5. In some cases, a saRNA may comprise a sequence or complementary sequence of MYF6. In some cases, a saRNA may comprise at least two sequences or complementary sequences of Mrf4, Pax7, PAX3, MYOG, MYF5, or MYF6. In some cases, a saRNA may comprise at least three sequences or complementary sequences of Mrf4, Pax7, PAX3, MYOG, MYF5, or MYF6. In some cases, a saRNA may comprise at least four sequences or complementary sequences of Mrf4, Pax7, PAX3, MYOG, MYF5, or MYF6. In some cases, a saRNA may comprise at least five sequences or complementary sequences of Mrf4, Pax7, PAX3, MYOG, MYF5, or MYF6. In some cases, a saRNA may comprise sequences or complementary sequences of Mrf4, Pax7, PAX3, MYOG, MYF5, and MYF6. In some cases, a nucleic acid molecule may comprise two or more different types of nucleic acid molecules. In some cases, two or more different types of nucleic acid molecules may comprise mRNA and siRNA. In some cases, two or more different types of nucleic acid molecules may comprise mRNA and miRNA. In some cases, two or more different types of nucleic acid molecules may comprise multiple mRNAs of different sequences. In some cases, the multiple sequences, each comprising a coding sequence and/or a non-coding sequence may be encoded within one saRNA construct. The mRNA may comprise the sequence of any one of MYOD1, MYOG, MYF5, MYF6, PAX3, PAX7, or any combination or variant thereof. The mRNA may comprise the sequence of any one of PPARY, adiponectin, FATP1-6, FABP4, GLUT4, Leptin, AdipoR1-2, and CD137.

In some cases, a siRNA may target POUF51 (OCT3/4.), KLF4, SOX2, or any combination or variant thereof. In some cases, two or more different types of nucleic acid molecules may comprise cDNA and siRNA. In some cases, a cDNA may comprise MYOD1, MYOG, MYF5, MYF6, PAX3, PAX7, or any combination or variant thereof. In some cases, two or more different nucleic acids may comprise an mRNA, cDNA, miRNA, transfer RNA, siRNA, unlocked RNA (uRNA), SaRNA, endless/circular RNA (eRNA), endless/circular RNA (eRNA), trans-amplifying RNA (ta-RNA), or any variant, combinations, or analogs thereof.

In some cases, one or more genes may be targeted and modulated with one, two, or a plurality of nucleic acid molecules. In some cases, one or more genes may comprise greater or equal to about 1, 2, 3, 4, 5, 6, 7, 8, 9, 10 genes, or more. In some cases, modulating expression of one or more genes in a cell may comprise enhancing expression of a first gene of the at least about two genes, and inhibiting expression of a second gene of the at least about two genes.

In some instances, a nucleic acid molecule may be modified. In some cases, a nucleic acid molecule may be chemically modified (i.e., the nucleic acid molecule comprise a chemical modification). In some instances, chemically modified nucleic acid molecule may reduce or inhibit degradation of the nucleic acid molecule at least about 5%, at least about 10%, at least about 15%, at least about 20%, at least about 25%, at least about 30%, at least about 35%, at least about 40%, at least about 45%, at least about 50%, at least about 55%, at least about 60%, at least about 65%, at least about 70%, at least about 75%, at least about 80%, at least about 85%, at least about 90%, at least about 95% or more, relative to a nucleic acid molecule not chemically modified.

In some cases, a chemically modified nucleic acid molecule may comprise an UNA or an LNA. In some instances, an LNA may have a structurally rigid modification. In some cases, an UNA may have a structurally flexible modification. In some cases, an UNA may comprise an acyclic analogue of RNA in which the bond between the C2′ and C3′ atoms of the ribose ring has been cleaved. In some cases, an LNA may comprise a modified nucleic acid molecule in which the ribose moiety is modified with an extra bridge connecting the 2′ oxygen and 4′ carbon. In some cases, a nucleic acid molecule may comprise an uRNA.

In some instances, a nucleic acid may have a length of at least about 5 nucleotides, 10 nucleotides, 50 nucleotides, 100 nucleotides, 500 nucleotides, 1000 nucleotides, 5000 nucleotides, 10000 nucleotides, or more. In some instances, a nucleic acid may have a length of at most about 5 nucleotides, 10 nucleotides, 50 nucleotides, 100 nucleotides, 500 nucleotides, 1000 nucleotides, 5000 nucleotides, or 10000 nucleotides.

### Polyplex

In some instances, a saccharide or a polyplex may facilitate an uptake of a nucleic acid by a cell. In some cases, a saccharide or a polyplex comprising a positive charge may form an ionic bond with a nucleic acid molecule. A nucleic acid molecule bound by a saccharide or associated into a polyplex may have a reduced negative charge. A nucleic acid molecule bound by a saccharide or associated into a polyplex may have a neutral charge. A nucleic acid molecule bound by a saccharide or associated into a polyplex may have a positive charge. The reduced negative charge, neutral charge, or positive charge of a nucleic acid molecule (and the saccharide or the polyplex) may facilitate its uptake by a cell. In some cases, a saccharide may bind a nucleic acid via coacervation. For example, reducing the net negative charge of a composition comprising a nucleic acid molecule-via (1) the binding or association of the nucleic acid molecule with a saccharide; (2) formation of a polyplex from the nucleic acid molecule and the saccharide; (3) a combination thereof—may facilitate the update of the nucleic acid molecule by the cell, wherein a plasma membrane may carry a negative charge. Association of a nucleic acid molecule with a saccharide or formation of a polyplex from the nucleic acid molecule and the saccharide may reduce the amount (number, weight, of mass) of the nucleic acid molecule to be used for contacting the cell (to allow for the cell to uptake the nucleic acid molecule), relative to the amount of the nucleic acid molecule to be used for contacting the cell without the saccharide or formation of the polyplex.

A nucleic acid molecule described herein may be present at a concentration at least about 1 picomolar (pM), 2 pM, 5 pM, 10 pM, 20 pM, 50 pM, 100 pM, 200 pM, 500 pM, 1 nanomolar (nM), 2 nM, 5 nM, 10 nM, 20 nM, 50 nM, 100 nM, 200 nM, 500 nM, 1 micromolar (μM), 2 μM, 5 μM, 10 μM, 20 μM, 50 M, 100 μM, 200 μM, 500 μM, 1 millimolar (mM), 2 mM, 5 mM, 10 mM, 20 mM, 50 mM, 100 mM, 200 mM, 500 mM, 1 molar (M) or more within the composition. A nucleic acid molecule described herein described herein may be present at a concentration at most about 1 pM, 2 pM, 5 pM, 10 pM, 20 pM, 50 pM, 100 pM, 200 pM, 500 pM, 1 nM, 2 nM, 5 nM, 10 nM, 20 nM, 50 nM, 100 nM, 200 nM, 500 nM, 1 μM, 2 μM, 5 μM, 10 μM, 20 μM, 50 μM, 100 μM, 200 μM, 500 μM, 1 mM, 2 mM, 5 mM, 10 mM, 20 mM, 50 mM, 100 mM, 200 mM, 500 mM, or 1 M within the composition.

In some instances, a saccharide and a nucleic acid molecule are configured to self-assemble with each other. In some cases, a saccharide and a nucleic acid molecule are configured to associate with each other. In some cases, a saccharide may bind a nucleic acid molecule. In some cases, a saccharide and a nucleic acid molecule are configured to form a polyplex. In some cases, the saccharide of a polyplex may comprise a modified saccharide or a lipid and/or anion polymer additive described thereof. In some instances, a saccharide and an mRNA are configured to self-assemble with each other. In some cases, a saccharide and an mRNA are configured to associate with each other. In some cases, a saccharide may bind an mRNA. In some cases, a saccharide and an mRNA are configured to form a polyplex. In some cases, a saccharide and an mRNA are configured to form a polyplex. In some instances, a saccharide and a miRNA are configured to self-assemble with each other. In some cases, a saccharide and a miRNA are configured to associate with each other. In some cases, a saccharide may bind a miRNA. In some cases, a saccharide and a miRNA are configured to form a polyplex. In some instances, a saccharide and a siRNA are configured to self-assemble with each other. In some cases, a saccharide and a siRNA are configured to associate with each other. In some cases, a saccharide may bind a siRNA. In some cases, a saccharide and a siRNA are configured to form a polyplex. In some instances, a chitosan and a nucleic acid molecule are configured to self-assemble with each other In some instances, a chitosan and a nucleic acid molecule are configured to associate with each other. In some cases, a chitosan may bind a nucleic acid molecule. In some cases, a chitosan and a nucleic acid molecule are configured to form a polyplex. In some instances, a chitosan and an mRNA are configured to self-assemble with each other. In some cases, a chitosan and an mRNA are configured to associate with each other. In some cases, a chitosan may bind an mRNA. In some cases, a chitosan and an mRNA are configured to form a polyplex. In some cases, a chitosan and an mRNA are configured to form a polyplex. In some instances, a chitosan and a miRNA are configured to self-assemble with each other. In some cases, a chitosan and a miRNA are configured to associate with each other. In some cases, a chitosan may bind a miRNA. In some cases, a chitosan and a miRNA are configured to form a polyplex. In some instances, a chitosan and a siRNA are configured to self-assemble with each other. In some cases, a chitosan and a siRNA are configured to associate with each other. In some cases, a chitosan may bind a siRNA. In some cases, a chitosan and a siRNA are configured to form a polyplex.

Self-assembly of two molecular entities may comprise the initiation and formation of a distinct molecular entity from the two molecular entities without an assistance of other molecular entity (e.g., a catalyst). In some cases, the nucleic acid molecule and the saccharide may associate with each other based at least on the electrostatic interaction between them. For example, the nucleic acid molecule may be anionic and the saccharide may be cationic. The electrostatic interaction between the anionic charge of the nucleic acid molecule and the cationic charge of the saccharide may facilitate the self-assembly of the nucleic acid molecule and the saccharide into a complex. The electrostatic interaction between the anionic charge of the nucleic acid molecule and the cationic charge of the saccharide may facilitate the formation of a polyplex of the nucleic acid molecule and the saccharide. Adjusting the charges of a saccharide (e.g., via modification, functionalization, or derivatization of the saccharide) may control the association, self-assembly, and/or formation of the polyplex between the saccharide and a nucleic acid molecule.

In some cases, a nucleic acid molecule is configured to be at or near a surface of a polyplex. The nucleic acid molecule is at or near the surface of the polyplex. A nucleic acid molecule is configured to be adsorbed onto a surface of the saccharides of a polyplex. The nucleic acid molecule may be adsorbed onto the saccharides of a surface a polyplex. In some cases, a nucleic acid molecule is configured to be encapsulated within a polyplex. A nucleic acid molecule may be encapsulated within a polyplex. In some cases, a nucleic acid molecule is present inside a polyplex. In some cases, a nucleic acid molecule is present outside a polyplex. In some cases, a nucleic acid molecule is present inside and outside a polyplex. The nucleic acid molecule may also be intercalated with other molecules, for example, the saccharide, of the polyplex. The configuration of the nucleic acid molecule and the saccharide of a polyplex may be random. The configuration of the nucleic acid molecule and the saccharide of a polyplex may be ordered. For example, the nucleic acid molecule and the saccharide of the polyplex may adopt an ordered configuration based on the electrostatic interaction between the nucleic acid molecule and the saccharide. In some cases, a polyplex is regularly shaped, irregularly shaped, spherical, linear, or branched. In some cases, a polyplex is regularly shaped, irregularly shaped or branched. In some cases, a polyplex is regularly shaped. In some cases, a polyplex is irregularly shaped. In some cases, a polyplex is branched. In some cases, a polyplex is spherical or linear. In some cases, a polyplex is spherical. In some cases, a polyplex is linear. A regularly shape may comprise any orthogonal shapes. A regularly shape may comprise any polygonal shapes (comprising 3, 4, 5, 6, 7, 8, 9, 10 or more edges). An irregular shape may comprise shapes that are not regular shapes.

In some instances, a size of a polyplex may be measured by a diameter of the polyplex. In some cases, a diameter of a polyplex may comprise an apparent diameter of the polyplex. In some cases, an apparent diameter of a polyplex may comprise an average diameter of a distribution of the diameters of a plurality of polyplexes. In some cases, an apparent diameter of a polyplex may comprise a representative diameter of a plurality of polyplexes. In some cases, an apparent diameter of a polyplex may comprise a mean diameter of a plurality of polyplexes. In some cases, an apparent diameter of a polyplex may comprise a median diameter of a plurality of polyplexes. In some cases, an apparent diameter of a polyplex may comprise a mode diameter of a plurality of polyplexes. The representative diameter may also comprise a quantile diameter or a range diameter of a plurality of polyplexes. The representative diameter may also be described as a distribution of a plurality of diameters of a plurality of polyplexes.

In some cases, a diameter of a polyplex may be measured by dynamic light scattering (DLS), gel filtration chromatography, analytical ultracentrifugation or microscopy. In some cases, a diameter of a polyplex formed in the compositions may be measured by a Dynamic light scattering (DLS). DLS may determine the size of a molecule (e.g., a polyplex) by measuring the random changes in the intensity of light scattered from a suspension or solution containing the molecule. DLS may measure the particle size by sensing the Brownian motion of particles. Since the Brownian motion velocities are higher for smaller particles, the Doppler spectral broadening of the scattered light may be size dependent. DLS may have a resolution of about 0.01 nanometer (nm), 0.1 nm, or 1 nm. In other instances, a polyplex formed is observed and/or measured by cryo-electron microscopy. In some cases, a polyplex may be loaded in the form of a thin membrane on a grid and frozen at −80° C., −90° C., −100° C., −110° C., −120° C., −130° C., −140° C., −150° C., −160° C., −170° C., −180° C., −190° C., −200° C. or lower temperature.

A polyplex may be maintained in a buffer. The buffer may have a pH of at least about 5.1, 5.2, 5.3, 5.4, 5.5, 5.6, 5.7, 5.8, 5.9, 6, 6.1, 6.2, 6.3, 6.4, 6.5, 6.6, 6.7, 6.8, 6.9, 7, 7.1, 7.2, 7.3, 7.4, 7.5, 7.6, 7.7, 7.8, 7.9, 8, 8.1, 8.2, 8.3, 8.4, 8.5, 8.6, 8.7, 8.8, 8.9, 9 or more. The buffer may have a pH of at most about 5.1, 5.2, 5.3, 5.4, 5.5, 5.6, 5.7, 5.8, 5.9, 6, 6.1, 6.2, 6.3, 6.4, 6.5, 6.6, 6.7, 6.8, 6.9, 7, 7.1, 7.2, 7.3, 7.4, 7.5, 7.6, 7.7, 7.8, 7.9, 8, 8.1, 8.2, 8.3, 8.4, 8.5, 8.6, 8.7, 8.8, 8.9, or 9. The buffer

The buffer may comprise a saline. The buffer may comprise phosphate-buffered saline (PBS). The buffer may also comprise HEPES or Tris-buffered saline (TBS).

A nucleic acid, a saccharide, a lipid, or a combination thereof may be incubated at about −80° C., −50° C., −20° C.,−10° C., −9° C., −8° C., −7° C., −6° C., −5° C., −4° C., −3° C., −2° C., −1° C., 0° C., 1° C., 2° C., 3° C., 4° C., 5° C., 6° C., 7° C., 8° C., 9° C., 10° C., 11° C., 12° C., 13° C., 14° C., 15° C., 16° C., 17° C., 18° C., 19° C., 20° C., 21° C., 22° C., 23° C., 24° C., 25° C., 26° C., 27° C., 28° C., 29° C., 30° C., 31° C., 32° C., 33° C., 34° C., 35° C., 36° C., 37° C. or higher to form a polyplex. A nucleic acid, a saccharide, a lipid, or a combination thereof may be incubated for at about 1 minute, 2 minutes, 3 minutes, 4 minutes, 5 minutes, 6 minutes, 7 minutes, 8 minutes, 9 minutes, 10 minutes, 11 minutes, 12 minutes, 13 minutes, 14 minutes, 15 minutes, 16 minutes, 17 minutes, 18 minutes, 19 minutes, 20 minutes, 21 minutes, 22 minutes, 23 minutes, 24 minutes, 25 minutes, 26 minutes, 27 minutes, 28 minutes, 29 minutes, 30 minutes, 1 hour, 2 hours, 3 hours, 4 hours, 5 hours or more to form a polyplex.

In some instances, an apparent diameter of a polyplex is at least about 5.0 nanometer (nm). In some instances, an apparent diameter of a polyplex is at least about 10.0 nm. In some instances, an apparent diameter of a polyplex is at least about 1.0 nm, at least about 1.5 nm, at least about 2.0 nm, at least about 2.5 nm, at least about 3.0 nm, at least about 3.5 nm, at least about 4.0 nm, at least about 4.5 nm, at least about 5.0 nm, at least about 5.5 nm, at least about 6.0 nm, at least about 6.5 nm, at least about 7.0 nm, at least about 7.5 nm, at least about 8.0 nm, at least about 8.5 nm, at least about 9.0, at least about 9.5 nm, at least about 10.0 nm, at least about 10.5 nm, at least about 11.0 nm, at least about 11.5 nm, at least about 12.0 or more. In some instances, an apparent diameter of a polyplex is at most about 5000 nm. In some instances, an apparent diameter of a polyplex is at most about 800 nm. In some instances, an apparent diameter of a polyplex is at most about 600 nm. In some instances, an apparent diameter of a polyplex is at most about 7000 nm, at most about 6000 nm, at most about 5000 nm, at most about 4000 nm, at most about 3000 nm, at most about 2000 nm, at most about 1000 nm, at most about 900 nm, at most about 800 nm, at most about 700 nm, at most about 600 nm, at most about 500 nm, at most about 400 nm, at most about 300 nm, at most about 200 nm, or at most about 100 nm.

In some instances, a composition described herein may comprise a nanoparticle. A nanoparticle may comprise a gold particle, a calcium phosphate nanoparticle, an iron oxide particle, a lipid nanoparticle, or a combination thereof. In some cases, a calcium phosphate (CaP) nanoparticle may comprise a silica shell, a PEI (poly(ethyleneimine)) shell, or a combination thereof. In some cases, calcium phosphate nanoparticle may comprise a triple shell calcium phosphate nanoparticle (CaP-nucleic acid-CaP-PEI), a calcium phosphate nanoparticle with a silica shell (CaP-nucleic acid-PEI-SiO2-SH), a calcium phosphate with a single-shell CaP-PEI, or a combination thereof (see, for example, Neuhaus B et al., RSV Adv., 2016, 6, 18102, which is herein incorporated by reference in its entirety).

In some instances, a polyplex may comprise a nanoparticle. In some cases, a nanoparticle may be at least about 5.0 nm. In some cases, a nanoparticle may be at least about 10.0 nm. In some cases, a nanoparticle may be at least about 1.0 nm, at least about 1.5 nm, at least about 2.0 nm, at least about 2.5 nm, at least about 3.0 nm, at least about 3.5 nm, at least about 4.0 nm, at least about 4.5 nm, at least about 5.0 nm, at least about 5.5 nm, at least about 6.0 nm, at least about 6.5 nm, at least about 7.0 nm, at least about 7.5 nm, at least about 8.0 nm, at least about 8.5 nm, at least about 9.0, at least about 9.5 nm, at least about 10.0 nm, at least about 10.5 nm, at least about 11.0 nm, at least about 11.5 nm, at least about 12.0 or more. In some cases, a nanoparticle may be at most about 10.0 nm. In some cases, a nanoparticle may be at most about 1.0 nm, at most about 1.5 nm, at most about 2.0 nm, at most about 2.5 nm, at most about 3.0 nm, at most about 3.5 nm, at most about 4.0 nm, at most about 4.5 nm, at most about 5.0 nm, at most about 5.5 nm, at most about 6.0 nm, at most about 6.5 nm, at most about 7.0 nm, at most about 7.5 nm, at most about 8.0 nm, at most about 8.5 nm, at most about 9.0, at most about 9.5 nm, at most about 10.0 nm, at most about 10.5 nm, at most about 11.0 nm, at most about 11.5 nm, or at most about 12.0.

In some cases, a nanoparticle may be a colloidal nanoparticle. The colloidal lipid particles used herein may be at least substantially non-toxic to a subject or a cell. In some cases, the lipid particles have a mean diameter of from about 40 nm to about 150 nm. In some cases, the diameter is from about 50 nm to about 140 nm, about 60 nm to about 130 nm, or to about 70 nm to about 120 nm. In some cases, a colloidal nanoparticle may be at most about 5000 nm. In some cases, a colloidal nanoparticle may be at most about 800 nm. In some cases, a colloidal nanoparticle may be at most about 600 nm. In some cases, a colloidal nanoparticle may be at most about 7000 nm, at most about 6000 nm, at most about 5000 nm, at most about 4000 nm, at most about 3000 nm, at most about 2000 nm, at most about 1000 nm, at most about 900 nm, at most about 800 nm, at most about 700 nm, at most about 600 nm, at most about 500 nm, at most about 400 nm, at most about 300 nm, at most about 200 nm, or at most about 100 nm. In some cases, a colloidal nanoparticle may be at least about 600 nm. In some cases, a colloidal nanoparticle may be at least about 7000 nm, at least about 6000 nm, at least about 5000 nm, at least about 4000 nm, at least about 3000 nm, at least about 2000 nm, at least about 1000 nm, at least about 900 nm, at least about 800 nm, at least about 700 nm, at least about 600 nm, at least about 500 nm, at least about 400 nm, at least about 300 nm, at least about 200 nm, or at least about 100 nm.

In some cases, the corresponding cationic charge density of a nanoparticle described herein may be at least about 0.5 mequiv/g, at least about 1 mequiv/g, at least about 1.5 mequiv/g, at least about 2 mequiv/g, at least about 2.5 mequiv/g, at least about 3 mequiv/g, at least about 3.5 mequiv/g, at least about 4 mequiv/g, at least about 4.5 mequiv/g, at least about 5 mequiv/g, at least about 5.5 mequiv/g, at least about 6 mequiv/g, at least about 6.5 mequiv/g, at least about 7 mequiv/g, at least about 7.5 mequiv/g, at least about 8 mequiv/g, at least about 8.5 mequiv/g, at least about 9 mequiv/g, at least about 9.5 mequiv/g, at least about 10 mequiv/g, at least about 10.5 mequiv/g, at least about 11 mequiv/g, at least about 11.5 mequiv/g, at least about 12 mequiv/g, at least about 12.5 mequiv/g, at least about 13 mequiv/g, at least about 13.5 mequiv/g, at least about 14 mequiv/g, at least about 14.5 mequiv/g, at least about 15 mequiv/g, at least about 15.5 mequiv/g, at least about 16 mequiv/g, at least about 16.5 mequiv/g, at least about 17 mequiv/g, at least about 17.5 mequiv/g, at least about 18 mequiv/g, at least about 18.5 mequiv/g, at least about 19 mequiv/g, at least about 19.5 mequiv/g, or at least about 20 mequiv/g. In some cases, the corresponding cationic charge density of a nanoparticle described herein may be at most about 0.5 mequiv/g, at most about 1 mequiv/g, at most about 1.5 mequiv/g, at most about 2 mequiv/g, at most about 2.5 mequiv/g, at most about 3 mequiv/g, at most about 3.5 mequiv/g, at most about 4 mequiv/g, at most about 4.5 mequiv/g, at most about 5 mequiv/g, at most about 5.5 mequiv/g, at most about 6 mequiv/g, at most about 6.5 mequiv/g, at most about 7 mequiv/g, at most about 7.5 mequiv/g, at most about 8 mequiv/g, at most about 8.5 mequiv/g, at most about 9 mequiv/g, at most about 9.5 mequiv/g, at most about 10 mequiv/g, at most about 10.5 mequiv/g, at most about 11 mequiv/g, at most about 11.5 mequiv/g, at most about 12 mequiv/g, at most about 12.5 mequiv/g, at most about 13 mequiv/g, at most about 13.5 mequiv/g, at most about 14 mequiv/g, at most about 14.5 mequiv/g, at most about 15 mequiv/g, at most about 15.5 mequiv/g, at most about 16 mequiv/g, at most about 16.5 mequiv/g, at most about 17 mequiv/g, at most about 17.5 mequiv/g, at most about 18 mequiv/g, at most about 18.5 mequiv/g, at most about 19 mequiv/g, at most about 19.5 mequiv/g, or at most about 20 mequiv/g. In some cases, the corresponding cationic charge density of a nanoparticle described herein may be from 0.005 to 2000 mequiv/g. In some cases, the corresponding cationic charge density of a nanoparticle described herein may be from 0.05 to 200 mequiv/g. In some cases, the corresponding cationic charge density of a nanoparticle described herein may be from 0.5 to 20 mequiv/g.

In some instances, a molar ratio of a polyplex may be measured. In some cases, the molar ratio of a polyplex may be a molar ratio of polycation amino groups of the polyplex to nucleic acid phosphate groups of the polyplex. In some cases, a molar ratio of a polyplex is at least about 1, at least about 2, at least about 3, at least about 4, at least about 5, at least about 6, at least about 7, at least about 8, at least about 9, at least about 10, at least about 15, at least about 20, at least about 25, at least about 30, at least about 35, at least about 40, at least about 45, at least about 50, at least about 51, at least about 52, at least about 53, at least about 54, at least about 55, at least about 56, at least about 57, at least about 58, at least about 59, or at least about 60. In some cases, a molar ratio of a polyplex is at most about 1, at most about 2, at most about 3, at most about 4, at most about 5, at most about 6, at most about 7, at most about 8, at most about 9, at most about 10, at most about 15, at most about 20, at most about 25, at most about 30, at most about 35, at most about 40, at most about 45, at most about 50, at most about 51, at most about 52, at most about 53, at most about 54, at most about 55, at most about 56, at most about 57, at most about 58, at most about 59, or at most about 60. In some cases, a molar ratio of a polyplex is at least about 1. In some cases, a molar ratio of a polyplex is at most about 60. In some cases, a molar ratio of a polyplex is from about 1 to about 60.

In some cases, a nanoparticles can be stored at about 4° C. In some cases, a nanoparticles can be stored at about −196° C., −150° C., −80° C., −50° C., −20° C., −15° C., −10° C., −5° C., 0° C., 1° C., 2° C., 3° C., 4° C., 5° C., 6° C., 7° C., 8° C., 9° C., 10° C., 15° C., 20° C., 25° C., 30° C., 37° C., 40° C. or higher. In some cases, a nanoparticles can be stored from about 7 top about 14 days. In some cases, a nanoparticles can be stored at about 1 day, 2 days, 3 days, 4 days, 5 days, 6 days, 7 days, 8 days, 9 days, 10 days, 11 days, 12 days, 13 days, 14 days, 1 month, 6 months, 1 year, or 5 years. In some cases, a nanoparticle may be lyophilized.

In some instances, a saccharide or a polyplex is configured to inhibit or reduce or inhibit degradation of a nucleic acid molecule. In some instances, a saccharide or a polyplex is configured to inhibit or reduce nuclease degradation of a nucleic acid molecule. In some cases, a saccharide (or the saccharide within a polyplex) may bind to a nucleic acid molecule to protect a nucleic acid against cleavage by nuclease. In some cases, a nuclease may comprise an exonuclease or an endonuclease. In some instances, a nuclease may comprise deoxyribonuclease or ribonuclease. In some cases, a nuclease may comprise topoisomerases, recombinases, ribozymes, and RNA splicing enzymes. In some cases, protection of nucleic acid molecules against degradation increases the amount of the nucleic acid molecules available for contacting the cell or being taken up by the cell.

In some cases, a saccharide or a polyplex may reduce or inhibit degradation of a nucleic acid molecule at least about 10%, relative to a nucleic acid molecule not bound by the saccharide or not associated with the polyplex. In some cases, a saccharide or a polyplex may reduce or inhibit degradation of a nucleic acid molecule at least about 50%, relative to a nucleic acid molecule not bound by the saccharide. In some cases, a saccharide may reduce or inhibit degradation of a nucleic acid molecule at least about 90%, relative to a nucleic acid molecule not bound by the saccharide or not associated with the polyplex. In some cases, a saccharide or polyplex may reduce or inhibit degradation of a nucleic acid molecule at least about 5%, at least about 10%, at least about 15%, at least about 20%, at least about 25%, at least about 30%, at least about 35%, at least about 40%, at least about 45%, at least about 50%, at least about 55%, at least about 60%, at least about 65%, at least about 70%, at least about 75%, at least about 80%, at least about 85%, at least about 90%, or at least about 95%, relative to a nucleic acid molecule not bound by the saccharide or not associated with the polyplex. In some cases, a saccharide or polyplex may reduce or inhibit degradation of a nucleic acid molecule at most about 10%, relative to a nucleic acid molecule not bound by the saccharide or not associated with the polyplex. In some cases, a saccharide or polyplex may reduce or inhibit degradation of a nucleic acid molecule at most about 50%, relative to a nucleic acid molecule not bound by the saccharide or not associated with the polyplex. In some cases, a saccharide or polyplex may reduce or inhibit degradation of a nucleic acid molecule at most about 90%. relative to a nucleic acid molecule not bound by the saccharide or not associated with the polyplex. In some cases, a saccharide or polyplex may reduce or inhibit degradation of a nucleic acid molecule at most about 5%, at most about 10%, at most about 15%, at most about 20%, at most about 25%, at most about 30%, at most about 35%, at most about 40%, at most about 45%, at most about 50%, at most about 55%, at most about 60%, at most about 65%, at most about 70%, at most about 75%, at most about 80%, at most about 85%, at most about 90%, at most about 95%, or 100% relative to a nucleic acid molecule not bound by the saccharide or not associated with the polyplex. The reduction or inhibition of degradation of a nucleic acid molecule may be measured by the amount of nucleic acid molecule present/absent or by the amount of the time it takes for a nucleic acid molecule to be degraded.

In some cases, a chitosan may reduce or inhibit degradation of a nucleic acid molecule. In some cases, a chitosan may be configured to stabilize a nucleic acid molecule. In some instances, the molecular weight of chitosan or the amine groups on the chain may results in low rates of enzymatic degradation. In some cases, the amine groups in chitosan may inhibit nuclease degradation of a nucleic acid. In some cases, the molecular weight of a chitosan may inhibit nuclease degradation of a nucleic acid. In some cases, a deacetylated chitosan may inhibit nuclease degradation of a nucleic acid.

In some cases, a chitosan may reduce or inhibit degradation of a nucleic acid molecule at least about 10%, relative to a nucleic acid molecule not bound by the chitosan. In some cases, a chitosan may reduce or inhibit degradation of a nucleic acid molecule at least about 50%, relative to a nucleic acid molecule not bound by the chitosan. In some cases, a chitosan may reduce or inhibit degradation of a nucleic acid molecule at least about 90%, relative to a nucleic acid molecule not bound by the chitosan. In some cases, a chitosan may reduce or inhibit degradation of a nucleic acid molecule at least about 5%, at least about 10%, at least about 15%, at least about 20%, at least about 25%, at least about 30%, at least about 35%, at least about 40%, at least about 45%, at least about 50%, at least about 55%, at least about 60%, at least about 65%, at least about 70%, at least about 75%, at least about 80%, at least about 85%, at least about 90%, or at least about 95%, relative to a nucleic acid molecule not bound by the chitosan. In some cases, a chitosan may reduce or inhibit degradation of a nucleic acid molecule at most about 10%, relative to a nucleic acid molecule not bound by the chitosan. In some cases, a chitosan may reduce or inhibit degradation of a nucleic acid molecule at most about 50%, relative to a nucleic acid molecule not bound by the chitosan. In some cases, a chitosan may reduce or inhibit degradation of a nucleic acid molecule at most about 90%, relative to a nucleic acid molecule not bound by the chitosan. In some cases, a chitosan may reduce or inhibit degradation of a nucleic acid molecule at most about 5%, at most about 10%, at most about 15%, at most about 20%, at most about 25%, at most about 30%, at most about 35%, at most about 40%, at most about 45%, at most about 50%, at most about 55%, at most about 60%, at most about 65%, at most about 70%, at most about 75%, at most about 80%, at most about 85%, at most about 90%, at most about 95%, or 100% relative to a nucleic acid molecule not bound by the chitosan.

### Lipids

In some cases, the compositions disclosed herein may comprise lipid. In some instances, a saccharide or a chitosan may be associated with a lipid (i.e., functionalized or derivatized with a lipid). In some cases, a saccharide or a chitosan may be linked to a lipid by a conjugation or chemical bond.

Lipids may facilitate update of a nucleic acid molecule by a cell. The cationic charges of the lipid may reduce or neutralize the anionic charge of the nucleic acid molecule and/or the plasma membrane of the cell, reducing the electrostatic repulsion of the nucleic acid molecule and the plasma membrane of the cell. In some case, lipids may also facilitate the endocytosis of the nucleic acid molecule by the cell.

In some cases, a nanoparticles may comprise a lipid. In some instances, a lipid may facilitate an uptake of a nucleic acid by a cell. In some cases, a lipid may comprise a liposome. In some cases, a nanoparticles may comprise a liposome, a polymer nanoparticle, and a lipid-nanoparticle. In some cases, a liposome is complexed with an associated nucleic acid for subsequent delivery of the nucleic acid to a cell or tissue. In some cases, a liposome may comprise cholesterol. In some cases, a lipid nanoparticle may condense and deliver various nuclei acid molecules to a cell. In some instances, a nanoparticle may comprise a solid lipid (i.e., lipid that remains solid at room temperature and body temperature) or a liquid lipid (i.e., oil, which remains liquid at room temperature and body temperature, for example, vegetable oil or a lipid extracted from human adipose tissue). In some instances, a nanoparticle may comprise a lipid layer (e.g., lipid monolayer) enclosing the nanoparticle core where a nucleic acid-cationic lipid is entrapped. In some cases, the lipid layer can be about 2 to 10 nm. In some cases, the lipid layer can be about 5 nm. In some instances, a lipid layer can be a lipid monolayer. In some cases, a lipid layer can comprise two or more lipid layers. In some instances, a lipid layer can be selected from: phospholipids such as lecithin, (L-α-phosphatidylcholine) or phosphatidylcholines with saturated and unsaturated fatty acids. In some cases, a lipid layer may also comprise other phospholipids, such as phosphatidic acids, phosphatidylethanolamines, phosphatidylglycerols, phosphatidylserines, phosphatidylinositols, cardiolipins, or a combination thereof. In some cases, a lipid layer can comprise lipid-PEG (lipid-polyethyleneglycol) conjugates with various molecular weight of PEG.

In some cases, a nanoparticle can be a solid lipid nanoparticle. In some cases, a solid lipid nanoparticle can comprise a lipid layer enclosing the nanoparticle core, where a solid lipid can be disposed in the nanoparticle core along with the nucleic acid-cationic lipid conjugate. In some instances, a solid lipid can be a lipid that remains a solid at the body temperature of an animal or cell, so that a solid lipid can be selected based on the animal or the cell. In some instances, a solid lipid can be selected from the following group: monoglycerides (e.g. glycerol monostearate), diglycerides (e.g., glycerol behenate), triglycerides (e.g., tristearin, trimyristin, trilaurin), waxes (e.g., acetyl palmitate), fatty acids (e.g., stearic acid, palmitic acid), steroids (e.g., cholesterol), and a combination thereof.

In some cases, a lipid particle further may comprise a PEG-lipid. The PEG-lipid can prevent the aggregation of particles. PEG may comprise a linear, water-soluble polymer of ethylene PEG repeating units with two terminal hydroxyl groups and are classified by their molecular weights. In some embodiments, the PEG moiety of the PEG-lipid may comprise an average molecular weight from about 600 to about 5,000 Da. In some cases, the average molecular weight is 2,000 Da. In some cases, the average molecular weight is 750 Da. In some instances, the PEG-lipid is selected from one or more of the following: PEG-Maleimide, PEG-PDP, PEG-Biotin, PEG-Amine, PEG-DBCO, PEG-Azide, PEG-Cyanur, PEG-Succinyl, PEG-Folate and/or PEG-Carboxylic acid. In some instances, the PEG-lipid is 1,2-DMG PEG 2000 (1,2 dimyristoyl-sn-glycero-3-phosphoethanolamine-N-[methoxy-(polyethylene glycol)-2000. In some instances, the PEG-lipid is a combination of 1,2-DMG PEG2000 and 1,3-DMG PEG2000 (1,3-DMG PEG2000 (1,3 dimyristoyl-sn-glycero-3-phosphoethanolamine-N-[methoxy-(polyethylene glycol)-2000), optionally in a 97:3 ratio. In some instances, the PEG-lipid is 1,2-DMG PEG 1000 (1,2 dimyristoyl-sn-glycero-3-phosphoethanolamine-N-[methoxy-(polyethylene glycol)-1000]. In some instances, the PEG-lipid is 1,2-DMG PEG 3000 (1,2 dimyristoyl-sn-glycero-3-phosphoethanolamine-N-[methoxy-(polyethylene glycol)-3000]. In some instances, the PEG-lipid may comprise from about 1% to about 10% of the total lipid present in the particle. In some cases, the sterol may comprise from about 1% to about 5%, 1% to about 3%, about 1% to about 2%, about 1% to 1.5% of the total lipid present in the particle. In some cases, the PEG-lipid may comprise 2% of the total lipid present. In some cases, the PEG-lipid may comprise 1.5% of the total lipid present. In other cases, the PEG-lipid may comprise from about 1% to about 5% of the total lipid present in the particle. In some cases, the PEG-lipid is 1,2-DMG PEG 2000 (1,2 dimyristoyl-sn-glycero-3-phosphoethanolamine-N-[methoxy-(polyethylene glycol)-2000 and may comprise from about 1% to about 2% of the total lipid present. In some instances, the PEG-lipid is a combination of 1,2-DMG PEG2000 and 1,3-DMG PEG2000 (1,3-DMG PEG2000 (1,3 dimyristoyl-sn-glycero-3-phosphoethanolamine-N-[methoxy-(polyethylene glycol)-2000), and may comprise from about 1% to about 2% of the total lipid present. In some instances, the PEG-lipid is 1,2-DMG PEG 1000 (1,2 dimyristoyl-sn-glycero-3-phosphoethanolamine-N-[methoxy-(polyethylene glycol)-1000], and may comprise from about 1 mol % to about 2 mol % of the total lipid present. In some instances, the PEG-lipid is 1,2-DMG PEG 3000 (1,2 dimyristoyl-sn-glycero-3-phosphoethanolamine-N-[methoxy-(polyethylene glycol)-3000], and may comprise from about 1% to about 2% of the total lipid present. In some instances, the lipid particle further may comprise a sterol.

In some instances, the sterol may be a cholesterol or a derivative thereof. Examples of cholesterol derivatives may comprise cholestanol, cholestanone, cholestenone, coprostanol, cholesteryl-2′-hydroxyethyl ether, cholesteryl-4′-hy-droxybutyl ether, and mixtures thereof. In some cases, the sterol may comprise from about 10% to about 50 mol % of the total lipid present in the particle. In some cases, the sterol may comprise from about 10% to about 20%, about 10% to about 15%, about 30% to about 40% of the total lipid present in the particle. In some cases, the sterol may comprise about 38% of the total lipid present. In other cases, the sterol may comprise from about 30 mol % to about 40 mol % of the total lipid present in the particle. In some cases, the sterol may be cholesterol or a derivative thereof and may comprise from about 30% to about 40% of the total lipid present. In some cases, the lipid particle further may comprise one or more stabilizing agents. Stabilizing agents ensure integrity of the lipid mixture. In some cases, the one or more stabilizing agents are polyethylene glycol-lipids. Suitable polyethylene glycol-lipids include PEG-modified phosphatidylethanolamine, PEG-modified phosphatidic acid, PEG-modified ceramides (e.g., PEG-CerC14 or PEG-CerC20), PEG-modified dialkylamines, PEG-modified diacylglycerols, PEG-modified alkylglycerols. Representative polyethylene glycol-lipids include PEG-c-DOMG, PEG-c-DMA, and PEG-s-DMG. In one embodiment, the polyethylene glycol-lipid may be N-[(methoxy poly(ethylene glycol) 2000) carbamyl]-1,2-dimyristyloxlpropyl-3-amine (PEG-c-DMA).

In some cases, a polyplex may comprise a cationic lipid. In other cases, a polyplex may comprise a cationic lipid and not a saccharide. In some cases, a polyplex may also comprise both a cationic lipid and a saccharide.

In some cases, a cationic lipid is selected from one or more of the following: 1,2-dilinoleyloxy-N,N-dimethylaminopropane (DLinDMA), 1,2-dilinolenyloxy-N,N-dimethylaminopropane (DLenDMA), dilinoleyl-4-(2-dimethylaminoethyl)-[1,3]-dioxolane (DLin-K-C2-DMA; “XTC2”), 2,2-dilinoleyl-4-(3-dimethylaminopropyl)-[1,3]-dioxolane (DLin-K-C3-DMA), 2,2-dilinoley 1-4-(4-dimethylamino buty 1)-[1,3]-dioxo lane (DLin-K-C4-DMA), 2,2-dilinoleyl-5-dimethylaminomethyl-[1,3]-dioxane (DLin-K6-DMA), 2,2-dilinoleyl-4-Nmethylpepiazino-[1,3]-dioxolane (DLin-K-MPZ), 2,2-dili-noleyl-4-dimethylaminomethyl-[1,3]-dioxolane (DLin-KDMA), 1,2-dilinoleylcarbamoyloxy-3-dimethylaminopropane (DLin-C-AP), 1,2-dilinoleyoxy-3-(dimethylamino acetoxypropane (DLin-DAC), 1,2-dilinoleyoxy-3-morpholinopropane (DLin-MA), 1,2-dilinoleoyl-3-dimethylaminopropane (DLinDAP), 1,2-dilinoley lthio-3-dimethylaminopropane (D Lin-S-D MA), 1-linoleoyl-2-linoleyloxy-3-dimethylaminopropane (DLin-2-D MAP), 1,2-dilinoley loxy-3-trimethy laminopropane chloride salt (DLin-TMA.C1), 1,2-dilinoleoyl-3-trimethy-laminopropane chloride salt (DLin-TAP.Cl), 1,2-dilinoleyloxy-3-(N-methylpiperazino) propane (DLin-MPZ), 3-(N,Ndilinoleylamino)-1,2-propanediol (DLinAP), 3-(N,Ndioleylamino)-1,2-propanedio (DOAP), 1,2-dilinoleyloxo-3-(2-N,N-dimethylamino) ethoxypropane (DLin-EG-DMA), N,N-dioleyl-N,N-dimethylammonium chloride (DO DAC), 1,2-dioleyloxy-N,N-dimethylaminopropane (DODMA), 1,2-disteary loxy-N,N-dimethy laminopropane (DSD MA), N-(1-(2,3-dioley loxy) propy 1)-N,N,N-trimethy ammonium chloride (DOTMA), N,N-distearyl-N,N-dimethylammonium bromide (DDAB), N-(1-(2,3-dioleoyloxy) propyl)-N,N,N-trimethylanunonium chloride (DOTAP), 3-(N,N′,N′dimethylaminoethane)-carbamoyl) cholesterol (DC-Chol), N-(1,2-dimyristyloxyprop-3-yl)-N,N-dimethyl-N-hydroxyethyl ammonium bromide (DMRIE), 2,3-dioleyloxy-N-[2 (spermine-carboxamido) ethy 1]-N,N-dimethy 1-1-propanaminiumtrifiuoroacetate (DOSPA). Dioctadecylamidoglycyl spermine (DOGS), 3-dimethylamino-2-(cholest-5-en-3-beta-oxybutan-4-oxy)-1-(cis,cis-9, 12-octadecadienoxy) propane (CLinDMA), 2-[5′-(cholest-5-en-3-beta-oxy)-3′-oxapentoxy)-3-dimethyl-1-(cis,cis-9′1-2′-octadecadienoxy) propane (CpLinDMA), N,N-dimethyl-3,4-dioleyloxybenzylamine (DMOBA), 1,2-N,N′dioleylcarbamyl-3-dimethylaminopropane (DOcarbDAP), 1,2-N,N′-dilinoleylcarbamyl-3-dimethy laminopropane (DLincarbDAP), or mixtures thereof. In some cases, a cationic lipid is selected from one or more of DOTAP (DOTAP methosulfate, N-(2,3-Dioleoyloxy-1-propyl) trimethylammonium methyl sulfate), DDA (Dimethyldioctadecylammonium (Bromide Salt)), DLin-KC2-DMA (KC2)(2-[2,2-bis [(9Z,12Z)-octadeca-9,12-dienyl]-1,3-dioxolan-4-yl]-N,N-dimethylethanamine), DLin-MC3-DMA (MC3) ((6Z,9Z,28Z,31Z)-heptatriacont-6,9,28,31-tetraene-19-yl 4-(dimethylamino) butanoate), cKK-E12 (3,6-bis(4-(bis(2-hydroxydodecyl)amino)butyl) piperazine-2,5-dione) and/or C12-200 (0.1,1′-((2-(4-(2-((2-(bis(2-hydroxydodecyl)amino)ethyl) (2-hydroxydodecyl)amino)ethyl) piperazin-1-yl)ethyl) azanediyl)bis(dodecan-2-ol). In some cases, a cationic lipid is selected from one or more of 1,2-diolelyloxy-3-(trimethylamino) propane (DOTAP); N-[1-(2,3,-ditetradecyloxy) propyl]-N,N-dimethyl-N-hydroxyethylammonium bromide (DMRIE); N-[1-(2,3,-dioleyloxy) propyl]-N,N-dimethyl-N-hydroxy ethylammonium bromide (DORIE); N-[1-(2,3-dioleyloxy) propyl]-N,N,N-trimethylammonium chloride (DOTMA); 3B [N-(N′,N′-dimethylaminoethane) carbamoly]cholesterol (DC-Chol); and dimethyldioctadecylammonium (DDAB). In some cases, the cationic lipid may be a neutral cationic lipid, that is, a lipid that at physiologic pH of 7.4 is predominantly, e.g., greater than 50%, neutral in charge but at a selected pH value less than physiologic pH tends to have a positive charge.

In some instances, the cationic lipid is DOTAP and may comprise 40 mole % (mol %) to 50 mol % of the total lipid present, the structural lipid is (1,2-distearoyl-sn-glycero-3-phosphorylcholine), SQDG (glycolipid), MGDG (monogalactosyldiacylglycerol)) (DSPC) and may comprise 10 mol of the total lipid present, and the lipid particle further may comprise cholesterol at 30 mol % to 40 mol % of the total lipid present, and one or more PEG-lipids at 1 mol to 2 mol % of the total lipid present. In some instances, the cationic lipid is DOTMA and may comprise 40 mol % to 50 mol % of the total lipid present, the structural lipid is DSPC and may comprise 10 mol % of the total lipid present, and the lipid particle further may comprise cholesterol at 30 mol % to 40 mol % of the total lipid present, and one or more PEG-lipids at 1 mol % to 2 mol % of the total lipid present.

### Conditions

In some cases, a method may comprise contacting a cell with a nucleic acid molecule, a saccharide, or a polymeric material in a condition sufficient for the cell to uptake the nucleic acid molecule, the saccharide, or the polymeric material. In some cases, the sufficient condition may comprise growing a cell in a 2D or 3D culture or environment. In some cases, the cell may be grown in the culture without a scaffold. In other cases, the cell may also be grown in the culture with a scaffold. In some cases, a cell may be viable. A cell may also be healthy. In some cases, a sufficient condition may comprise a population of viable cells before the contacting. The population of cells may comprise at least about 10%, 20%, 30%, 40%, 50%, 60%, 70%, 80%, 90%, 99% or more viable cells. The population of cells may also comprise from about 80% to about 90% viable cells. The population of cells may comprise at most about 10%, 20%, 30%, 40%, 50%, 60%, 70%, 80%, 90%, or 100% viable cells.

In some cases, a polyplex may be prepared immediately prior to the contacting. In some cases, a polyplex may be prepared at most about 5 hours, 4 hours, 3 hours, 2 hours, 1 hour, 45 minutes, 30 minutes, 15 minutes, or 5 minutes before the contacting. In some cases, a nucleic acid molecule may be kept at a temperature at most about −196° C., −150° C., −80° C., −50° C., −20° C., −15° C., −10° C., −5° C., 0° C., 1° C., 2° C., 3° C., 4° C., 5° C., 6° C., 7° C., 8° C., 9° C., 10° C. prior to the formation of the polyplex.

In some cases, a sufficient condition may comprise contacting a cell with a nucleic acid molecule, a saccharide, or a combination thereof for at least about 1, 2, 3, 4, 5, 6, 7, 8, 9, 10, 11, 12, 13, 14, 15, 16, 17, 18, 19, 20, 21, 22, 23, 24 hours or more. In some cases, a sufficient condition may comprise contacting a cell with a nucleic acid molecule, a saccharide, or a combination thereof for at least about 1, 2, 3, 4, 5 days or more. In some cases, the contacting may be carried out in a 2D or 3D culture condition. In some cases, the cell may be cultured in a first media for at least about 1, 2, 3, 4, 5, 6, 7, 8, 9, 10, 11, 12, 13, 14, 15, 16, 17, 18, 19, 20, 21, 22, 23, or 24 hours after the contacting. In some cases, the cell may be cultured in a first media for at least about 1, 2, 3, 4, 5 days after the contacting. In some cases, the cell may be cultured in a second media at least about 1, 2, 3, 4, 5, 6, 7, 8, 9, 10, 11, 12, 13, 14, 15, 16, 17, 18, 19, 20, 21, 22, 23, or 24 hours after the cell is cultured in the first media. In some cases, the cell may be cultured in a second media at least about 1, 2, 3, 4, 5 days after the cell is cultured in the first media. In some cases, the cell may be cultured in a second media for at least about 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96 hours or more. In some cases, the cell may be cultured in a second media for at least about 5, 6, 7, 8, 9, 10 days or more. In some cases, the first media may be the same as the second media. In some cases, the first media may be different from the second media. In some cases, the first or the second media may be any media described herein.

A sufficient condition may comprise contacting or incubating the cell, the saccharide, the nucleic acid molecule, the polymeric material, or a combination thereof with at most about 25%, 24%, 23%, 22%, 21%, 20%, 19%, 18%, 17%, 16%, 15%, 14%, 13%, 12%, 11%, 10%, 9%, 8%, 7%, 6%, 5%, 4%, 3%, 2%, 1% or lower atmospheric oxygen. A sufficient condition may comprise contacting or incubating the cell, the saccharide, the nucleic acid molecule, the polymeric material, or a combination thereof with at least about 1%, 2%, 3%, 4%, 5%, 6%, 7%, 8%, 9%, 10%, 11%, 12%, 13%, 14%, 15%, 16%, 17%, 18%, 19%, 20%, 21%, 22%, 23%, 24%, 25% or more atmospheric oxygen.

A sufficient condition may comprise contacting or incubating the cell, the saccharide, the nucleic acid molecule, the polymeric material, or a combination thereof with at most about 1%, 1.5%, 2%, 2.5%, 3%, 3.5%, 4%, 4.5%, 5%, 5.5%, 6%, 6.5%, 7%, 7.5%, 8%, 8.5%, 9%, 9.5%, 10%, 10.5%, 11%, 11.5%, 12%, 12.5%, 13%, 13.5%, 14%, 14.5%, or 15% atmospheric carbon dioxide (CO₂).

The methods provided herein may comprise contacting or incubating the cell, the saccharide, the nucleic acid molecule, the polymeric material, or a combination thereof within a cell culture container. The cell culture container may comprise a 35 mm culture dish, a 60 mm culture dish, a 100 mm culture dish, a 150 mm culture dish, a 6-well culture plate, a 12-well culture plate, a 24-well culture plate, a 48-well culture plate, a 96-well culture plate, a T-25 flask, a T-75 flask, a T-175 flask, or a T-225 flask.

The cell culture container may have a cell growth surface area. The cell growth surface area may have an area of about 0.1 square centimeter (cm\hat{ }2), 0.2 cm\hat{ }2, 0.3 cm\hat{ }2, 0.4 cm\hat{ }2, 0.5 cm\hat{ }2, 0.6 cm\hat{ }2, 0.7 cm\hat{ }2, 0.8 cm\hat{ }2, 0.9 cm\hat{ }2, 1 cm\hat{ }2, 1.1 cm\hat{ }2, 1.2 cm\hat{ }2, 1.3 cm\hat{ }2, 1.4 cm\hat{ }2, 1.5 cm\hat{ }2, 1.6 cm\hat{ }2, 1.7 cm\hat{ }2, 1.8 cm\hat{ }2, 1.9 cm\hat{ }2, 2 cm\hat{ }2, 2.1 cm\hat{ }2, 2.2 cm\hat{ }2, 2.3 cm\hat{ }2, 2.4 cm\hat{ }2, 2.5 cm\hat{ }2, 2.6 cm\hat{ }2, 2.7 cm\hat{ }2, 2.8 cm\hat{ }2, 2.9 cm\hat{ }2, 3 cm\hat{ }2, 3.1 cm\hat{ }2, 3.2 cm\hat{ }2, 3.3 cm\hat{ }2, 3.4 cm\hat{ }2, 3.5 cm\hat{ }2, 3.6 cm\hat{ }2, 3.7 cm\hat{ }2, 3.8 cm\hat{ }2, 3.9 cm\hat{ }2, 4 cm\hat{ }2, 4.1 cm\hat{ }2, 4.2 cm\hat{ }2, 4.3 cm\hat{ }2, 4.4 cm\hat{ }2, 4.5 cm\hat{ }2, 4.6 cm\hat{ }2, 4.7 cm\hat{ }2, 4.8 cm\hat{ }2, 4.9 cm\hat{ }2, 5 cm\hat{ }2, 6 cm\hat{ }2, 7 cm\hat{ }2, 8 cm\hat{ }2, 9 cm\hat{ }2, 10 cm\hat{ }2, 20 cm\hat{ }2, 30 cm\hat{ }2, 40 cm\hat{ }2, 50 cm\hat{ }2, 60 cm\hat{ }2, 70 cm\hat{ }2, 80 cm\hat{ }2, 90 cm\hat{ }2, 100 cm\hat{ }2, 110 cm\hat{ }2, 120 cm\hat{ }2, 130 cm\hat{ }2, 140 cm\hat{ }2, 150 cm\hat{ }2, 200 cm\hat{ }2, 500 cm\hat{ }2, 1000 cm\hat{ }2 or more.

The methods provided herein may comprise contacting a cell population with a nucleic acid molecule, a saccharide, a polymeric material, or a combination thereof. The cell population may comprise at least about 1×10 cm\hat{ }3 cells, 2×10 cm\hat{ }3 cells, 3×10 cm\hat{ }3 cells, 4×10 cm\hat{ }3 cells, 5×10 cm\hat{ }3 cells, 6×10 cm\hat{ }3 cells, 7×10 cm\hat{ }3 cells, 8×10 cm\hat{ }3 cells, 9×10 cm\hat{ }3 cells, 1×10 cm\hat{ }4 cells, 2×10 cm\hat{ }4 cells, 3×10 cm\hat{ }4 cells, 4×10 cm\hat{ }4 cells, 5×10 cm\hat{ }4 cells, 6×10 cm\hat{ }4 cells, 7×10 cm\hat{ }4 cells, 8×10 cm\hat{ }4 cells, 9×10 cm\hat{ }4 cells, 1×10 cm\hat{ }5 cells, 2×10 cm\hat{ }5 cells, 3×10 cm\hat{ }5 cells, 4×10 cm\hat{ }5 cells, 5×10 cm\hat{ }5 cells, 6×10 cm\hat{ }5 cells, 7×10 cm\hat{ }5 cells, 8×10 cm\hat{ }5 cells, 9×10 cm\hat{ }5 cells, 1×10 cm\hat{ }6 cells, 2×10 cm\hat{ }6 cells, 3×10 cm\hat{ }6 cells, 4×10 cm\hat{ }6 cells, 5×10 cm\hat{ }6 cells, 6×10 cm\hat{ }6 cells, 7×10 cm\hat{ }6 cells, 8×10 cm\hat{ }6 cells, 9×10 cm\hat{ }6 cells, 1×10 cm\hat{ }7 cells, 2×10 cm\hat{ }7 cells, 3×10 cm\hat{ }7 cells, 4×10 cm\hat{ }7 cells, 5×10 cm\hat{ }7 cells, 6×10 cm\hat{ }7 cells, 7×10 cm\hat{ }7 cells, 8×10 cm\hat{ }7 cells, 9×10 cm\hat{ }7 cells, 1×10 cm\hat{ }8 cells, 2×10 cm\hat{ }8 cells, 3×10 cm\hat{ }8 cells, 4×10 cm\hat{ }8 cells, 5×10 cm\hat{ }8 cells, 6×10 cm\hat{ }8 cells, 7×10 cm\hat{ }8 cells, 8×10 cm\hat{ }8 cells, 9×10 cm\hat{ }8 or more cells. The cell population may comprise at most about 1×10 cm\hat{ }3 cells, 2×10 cm\hat{ }3 cells, 3×10 cm\hat{ }3 cells, 4×10 cm\hat{ }3 cells, 5×10 cm\hat{ }3 cells, 6×10 cm\hat{ }3 cells, 7×10 cm\hat{ }3 cells, 8×10 cm\hat{ }3 cells, 9×10 cm\hat{ }3 cells, 1×10 cm\hat{ }4 cells, 2×10 cm\hat{ }4 cells, 3×10 cm\hat{ }4 cells, 4×10 cm\hat{ }4 cells, 5×10 cm\hat{ }4 cells, 6×10 cm\hat{ }4 cells, 7×10 cm\hat{ }4 cells, 8×10 cm\hat{ }4 cells, 9×10 cm\hat{ }4 cells, 1×10 cm\hat{ }5 cells, 2×10 cm\hat{ }5 cells, 3×10 cm\hat{ }5 cells, 4×10 cm\hat{ }5 cells, 5×10 cm\hat{ }5 cells, 6×10 cm\hat{ }5 cells, 7×10 cm\hat{ }5 cells, 8×10 cm\hat{ }5 cells, 9×10 cm\hat{ }5 cells, 1×10 cm\hat{ }6 cells, 2×10 cm\hat{ }6 cells, 3×10 cm\hat{ }6 cells, 4×10 cm\hat{ }6 cells, 5×10 cm\hat{ }6 cells, 6×10 cm\hat{ }6 cells, 7×10 cm\hat{ }6 cells, 8×10 cm\hat{ }6 cells, 9×10 cm\hat{ }6 cells, 1×10 cm\hat{ }7 cells, 2×10 cm\hat{ }7 cells, 3×10 cm\hat{ }7 cells, 4×10 cm\hat{ }7 cells, 5×10 cm\hat{ }7 cells, 6×10 cm\hat{ }7 cells, 7×10 cm\hat{ }7 cells, 8×10 cm\hat{ }7 cells, 9×10 cm\hat{ }7 cells, 1×10 cm\hat{ }8 cells, 2×10 cm\hat{ }8 cells, 3×10 cm\hat{ }8 cells, 4×10 cm\hat{ }8 cells, 5×10 cm\hat{ }8 cells, 6×10 cm\hat{ }8 cells, 7×10 cm\hat{ }8 cells, 8×10 cm\hat{ }8 cells, or 9×10 cm\hat{ }8 cells.

The sufficient conditions for the methods provide herein may comprise a desirable cell density. The desirable cell density may be at least about 1×10 cm\hat{ }2 cells per cm\hat{ }2 cell growth area, 2.5×10 cm\hat{ }2 cells per cm\hat{ }2 cell growth area, 5×10 cm\hat{ }2 cells per cm\hat{ }2 cell growth area, 1×10 cm\hat{ }3 cells per cm\hat{ }2 cell growth area, 2.5×10 cm\hat{ }3 cells per cm\hat{ }2 cell growth area, 5×10 cm\hat{ }3 cells per cm\hat{ }2 cell growth area, 1×10 cm\hat{ }4 cells per cm\hat{ }2 cell growth area, 2.5×10 cm\hat{ }4 cells per cm\hat{ }2 cell growth area, 5×10 cm\hat{ }4 cells per cm\hat{ }2 cell growth area, 1×10 cm\hat{ }5 cells per cm\hat{ }2 cell growth area, 2.5×10 cm\hat{ }5 cells per cm\hat{ }2 cell growth area, 5×10 cm\hat{ }5 cells per cm\hat{ }2 cell growth area, 1×10 cm\hat{ }6 cells per cm\hat{ }2 cell growth area, 2.5×10 cm\hat{ }6 cells per cm\hat{ }2 cell growth area, 5×10 cm\hat{ }6 cells per cm\hat{ }2 cell growth area, 1×10 cm\hat{ }7 cells per cm\hat{ }2 cell growth area, 2.5×10 cm\hat{ }7 cells per cm\hat{ }2 cell growth area, 5×10 cm\hat{ }7 cells per cm\hat{ }2 cell growth area, 1×10 cm\hat{ }8 cells per cm\hat{ }2 cell growth area, 2.5×10 cm\hat{ }8 cells per cm\hat{ }2 cell growth area, 5×10 cm\hat{ }8 cells per cm\hat{ }2 cell growth area or more. The desirable cell density may be at most about 1×10 cm\hat{ }2 cells per cm\hat{ }2 cell growth area, 2.5×10 cm\hat{ }2 cells per cm\hat{ }2 cell growth area, 5×10 cm\hat{ }2 cells per cm\hat{ }2 cell growth area, 1×10 cm\hat{ }3 cells per cm\hat{ }2 cell growth area, 2.5×10 cm\hat{ }3 cells per cm\hat{ }2 cell growth area, 5×10 cm\hat{ }3 cells per cm\hat{ }2 cell growth area, 1×10 cm\hat{ }4 cells per cm\hat{ }2 cell growth area, 2.5×10 cm\hat{ }4 cells per cm\hat{ }2 cell growth area, 5×10 cm\hat{ }4 cells per cm\hat{ }2 cell growth area, 1×10 cm\hat{ }5 cells per cm\hat{ }2 cell growth area, 2.5×10 cm\hat{ }5 cells per cm\hat{ }2 cell growth area, 5×10 cm\hat{ }5 cells per cm\hat{ }2 cell growth area, 1×10 cm\hat{ }6 cells per cm\hat{ }2 cell growth area, 2.5×10 cm\hat{ }6 cells per cm\hat{ }2 cell growth area, 5×10 cm\hat{ }6 cells per cm\hat{ }2 cell growth area, 1×10 cm\hat{ }7 cells per cm\hat{ }2 cell growth area, 2.5×10 cm\hat{ }7 cells per cm\hat{ }2 cell growth area, 5×10 cm\hat{ }7 cells per cm\hat{ }2 cell growth area, 1×10 cm\hat{ }8 cells per cm\hat{ }2 cell growth area, 2.5×10 cm\hat{ }8 cells per cm\hat{ }2 cell growth area, or 5×10 cm\hat{ }8 cells per cm\hat{ }2 cell growth area.

In some cases, a nucleic acid and a saccharide, when used for contacting or transfecting a cell, may have a mass ratio of at least about 10000:1, 5000:1, 2000:1, 1000:1, 500:1, 200:1, 100:1, 50:1, 20:1, 10:1, 5:1, 2:1, 1:1, 1:2, 1:5, 1:10, 1:20, 1:50, 1:100, 1:200, 1:500, 1:1000, 1:2000, 1:5000, or 1:10000. In some cases, a nucleic acid and a saccharide, when used for contacting or transfecting a cell, may have a mass ratio of at most about 10000:1, 5000:1, 2000:1, 1000:1, 500:1, 200:1, 100:1, 50:1, 20:1, 10:1, 5:1, 2:1, 1:1, 1:2, 1:5, 1:10, 1:20, 1:50, 1:100, 1:200, 1:500, 1:1000, 1:2000, 1:5000, or 1:10000. In some cases, a nucleic acid and a lipid, when used for contacting or transfecting a cell, may have a mass ratio of at least about 10000:1, 5000:1, 2000:1, 1000:1, 500:1, 200:1, 100:1, 50:1, 20:1, 10:1, 5:1, 2:1, 1:1, 1:2, 1:5, 1:10, 1:20, 1:50, 1:100, 1:200, 1:500, 1:1000, 1:2000, 1:5000, or 1:10000. In some cases, a nucleic acid and a lipid, when used for contacting or transfecting a cell, may have a mass ratio of at most about 10000:1, 5000:1, 2000:1, 1000:1, 500:1, 200:1, 100:1, 50:1, 20:1, 10:1, 5:1, 2:1, 1:1, 1:2, 1:5, 1:10, 1:20, 1:50, 1:100, 1:200, 1:500, 1:1000, 1:2000, 1:5000, or 1:10000. In some cases, a nucleic acid, a saccharide, and a lipid, when used for contacting or transfecting a cell, may have a mass ratio of about 1:2:1, 1:2:,2, 1:2:3, 1:2:4, 1:2:5, 1:2:10, 1:2:20, 1:4:4, 1:10:10, 1:10:100, 1:100:100, 1:300:300, 1:350:300, 1:400:300, 1:450:300, 1:500:300; 1:550:300, 1:600:300, 1:650:300, 1:700:300, 1:1000:300, 5:10:100, 5:100:100, 5:300:300, 5:350:300, 5:400:300, 5:450:300, 5:500:300; 5:550:300, 5:600:300, 5:650:300, 5:700:300, 5:1000:300, 10:10:100, 10:100:100, 10:300:300, 10:350:300, 10:400:300, 10:450:300, 10:500:300; 10:550:300, 10:600:300, 10:650:300, 10:700:300, 10:1000:300, 1:500:1, 1:500:10, 1:500:100, 1:500:150, 1:500:200, 1:500:250, 1:500:300, 1:500:350, 1:500:400, 1:500:450, or 1:500:500.

In some cases, a nucleic acid may be at least about 0.001 ng/10000 cells, 0.002 ng/10000 cells, 0.005 ng/10000 cells, 0.01 ng/10000 cells, 0.02 ng/10000 cells, 0.05 ng/10000 cells, 0.1 ng/10000 cells, 0.2 ng/10000 cells, 0.5 ng/10000 cells, 1 ng/10000 cells, 2 ng/10000 cells, 5 ng/10000 cells, 10 ng/10000 cells, 20 ng/10000 cells, 50 ng/10000 cells, 100 ng/10000 cells, 200 ng/10000 cells, 500 ng/10000 cells, 1000 ng/10000 cells, 5000 ng/10000 cells, 10000 ng/10000 cells or more, when used for contacting or transfecting a cell. In some cases, a nucleic acid may be at most about 0.001 ng/10000 cells, 0.002 ng/10000 cells, 0.005 ng/10000 cells, 0.01 ng/10000 cells, 0.02 ng/10000 cells, 0.05 ng/10000 cells, 0.1 ng/10000 cells, 0.2 ng/10000 cells, 0.5 ng/10000 cells, 1 ng/10000 cells, 2 ng/10000 cells, 5 ng/10000 cells, 10 ng/10000 cells, 20 ng/10000 cells, 50 ng/10000 cells, 100 ng/10000 cells, 200 ng/10000 cells, 500 ng/10000 cells, 1000 ng/10000 cells, 5000 ng/10000 cells, or 10000 ng/10000 cells, when used for contacting or transfecting a cell.

In some cases, a saccharide may be at least about 0.001 ng/10000 cells, 0.002 ng/10000 cells, 0.005 ng/10000 cells, 0.01 ng/10000 cells, 0.02 ng/10000 cells, 0.05 ng/10000 cells, 0.1 ng/10000 cells, 0.2 ng/10000 cells, 0.5 ng/10000 cells, 1 ng/10000 cells, 2 ng/10000 cells, 5 ng/10000 cells, 10 ng/10000 cells, 20 ng/10000 cells, 50 ng/10000 cells, 100 ng/10000 cells, 200 ng/10000 cells, 500 ng/10000 cells, 1000 ng/10000 cells, 2000 ng/10000 cells, 5000 ng/10000 cells, 10000 ng/10000 cells, 20 μg/10000 cells, 50 μg/10000 cells, 100 μg/10000 cells, 200 μg/10000 cells, 500 μg/10000 cells, 1000 μg/10000 cells, 2000 μg/10000 cells, 5000 μg/10000 cells, 10000 μg/10000 cells or more, when used for contacting or transfecting a cell. In some cases, a saccharide may be at most about 0.001 ng/10000 cells, 0.002 ng/10000 cells, 0.005 ng/10000 cells, 0.01 ng/10000 cells, 0.02 ng/10000 cells, 0.05 ng/10000 cells, 0.1 ng/10000 cells, 0.2 ng/10000 cells, 0.5 ng/10000 cells, 1 ng/10000 cells, 2 ng/10000 cells, 5 ng/10000 cells, 10 ng/10000 cells, 20 ng/10000 cells, 50 ng/10000 cells, 100 ng/10000 cells, 200 ng/10000 cells, 500 ng/10000 cells, 1000 ng/10000 cells, 2000 ng/10000 cells, 5000 ng/10000 cells, 10000 ng/10000 cells, 20 μg/10000 cells, 50 μg/10000 cells, 100 μg/10000 cells, 200 μg/10000 cells, 500 μg/10000 cells, 1000 μg/10000 cells, 2000 μg/10000 cells, 5000 μg/10000 cells, or 10000 μg/10000 cells, when used for contacting or transfecting a cell.

In some cases, a lipid may be at least about 0.001 ng/10000 cells, 0.002 ng/10000 cells, 0.005 ng/10000 cells, 0.01 ng/10000 cells, 0.02 ng/10000 cells, 0.05 ng/10000 cells, 0.1 ng/10000 cells, 0.2 ng/10000 cells, 0.5 ng/10000 cells, 1 ng/10000 cells, 2 ng/10000 cells, 5 ng/10000 cells, 10 ng/10000 cells, 20 ng/10000 cells, 50 ng/10000 cells, 100 ng/10000 cells, 200 ng/10000 cells, 500 ng/10000 cells, 1000 ng/10000 cells, 2000 ng/10000 cells, 5000 ng/10000 cells, 10000 ng/10000 cells, 20 μg/10000 cells, 50 μg/10000 cells, 100 μg/10000 cells, 200 μg/10000 cells, 500 μg/10000 cells, 1000 μg/10000 cells, 2000 μg/10000 cells, 5000 μg/10000 cells, 10000 μg/10000 cells or more, when used for contacting or transfecting a cell. In some cases, a lipid may be at most about 0.001 ng/10000 cells, 0.002 ng/10000 cells, 0.005 ng/10000 cells, 0.01 ng/10000 cells, 0.02 ng/10000 cells, 0.05 ng/10000 cells, 0.1 ng/10000 cells, 0.2 ng/10000 cells, 0.5 ng/10000 cells, 1 ng/10000 cells, 2 ng/10000 cells, 5 ng/10000 cells, 10 ng/10000 cells, 20 ng/10000 cells, 50 ng/10000 cells, 100 ng/10000 cells, 200 ng/10000 cells, 500 ng/10000 cells, 1000 ng/10000 cells, 2000 ng/10000 cells, 5000 ng/10000 cells, 10000 ng/10000 cells, 20 μg/10000 cells, 50 μg/10000 cells, 100 μg/10000 cells, 200 μg/10000 cells, 500 μg/10000 cells, 1000 μg/10000 cells, 2000 μg/10000 cells, 5000 μg/10000 cells, or 10000 μg/10000 cells, when used for contacting or transfecting a cell.

Any compositions herein may be biodegradable. Any compositions herein may be biocompatible with a cell that it contacts. A composition, saccharide, lipid, nucleic acid molecule, polyplex, polymeric material, or a combination thereof, described herein, may be biodegradable or biocompatible with a cell. Biocompatible compositions, saccharides, chitosans, lipids, nucleic acid molecules, polyplexes, polymeric materials, or a combination thereof may be beneficial. Biocompatible materials may have immunostimulatory activity, anticoagulant activity, wound-healing properties, anti-microbial properties, or a combination thereof. In some cases, biocompatible composition, saccharide, chitosan, lipid, nucleic acid molecule, polyplex, polymeric material, or a combination thereof may be non-toxic, non-hemolytic, non-immunogenic, slowly biodegradable, and/or nuclease resistant. In some cases, a saccharide or chitosan may increase transcellular and paracellular transport. Thus, using any biocompatible materials described herein may increase the number of cells that has taken up any nucleic acids described herein, encapsulated by the polymeric materials described herein, or a combination thereof, relative to using non-biocompatible materials.

Biodegradable compositions, saccharides, chitosans, lipids, nucleic acid molecules, polyplexes, polymeric materials, or a combination thereof may be beneficial. For example, even if a biodegradable material may have undesirable effects to a cell (cytotoxicity, carcinogenic activities, immunogenic activities, or a combination thereof), since the material is biodegradable, the undesirable effects generated by the material is minimized, relative to the non-biodegradable counterparts. Biodegradability may be mediated by various enzymes of the cell.

### Protein Expression or Differentiation

In some instances, a saccharide, a nucleic acid molecule, or a combination thereof may be configured to facilitate protein expression of a cell. A saccharide, a nucleic acid molecule, or a combination thereof may facilitate alteration of protein expression of a cell. A nucleic acid molecule, or a combination thereof may alter the protein expression of a cell. In some instances, a nucleic acid molecule, or a combination thereof may be configured to alter the protein expression of a cell. In some instances, a nucleic acid molecule, or a combination thereof may be configured to promote differentiation of a cell. In some instances, a nucleic acid molecule, or a combination thereof may be configured to facilitate differentiation of a cell. In some instances, a saccharide and a nucleic acid molecule are configured to facilitate or alter protein expression, promote differentiation, or a combination thereof of a cell (i.e., the saccharide and the nucleic acid molecule are collectively configured to facilitate or alter protein expression, promote differentiation, or a combination thereof of the cell). The terms “collectively” or grammatically equivalent, when used herein referring to the impact of a group of molecular entities on a cellular process (e.g., the group of molecular entities collectively impact the cellular process), refers to the impact generated by the group of molecular entities.

In some instances, a polymeric material may be configured to encapsulate a cell. Encapsulation of the cell, in some cases, may facilitate and/or promote differentiation of a cell. Encapsulation of the cell, in some cases, may also facilitate or promote the alternation of the protein expression of the cell.

In some instances, an alteration in protein expression within a cell may initiate or facilitate the process of a conversion of a cell type into another cell type. Initiation or alteration of protein expression within a cell may alter the shared structural or functional characteristics of a cell, leading to conversion of a cell type into another cell type. In some case, a change in protein expression within a cell may facilitate or promote differentiation of the cell. In some instances, protein expression may result in a differentiation which may comprise transdifferentiation. In some cases, a transdifferentiation may comprise a differentiation of a cell type into another cell type without reaching a pluripotent cell state during the differentiation process. In some instances, a differentiation may comprise transdifferentiation of somatic cells or directed differentiation of naive cells.

In an aspect, the present disclosure provides a method for altering the protein expression of a cell that may result in cell differentiation. In some instances, a method may comprise contacting a cell with a composition comprising a saccharide or a nucleic acid molecule, and encapsulating a cell and a composition using a polymeric material, thereby facilitating differentiation of a cell. In some cases, a polymeric material may facilitate encapsulation of a cell. In some cases, a polymeric material may encapsulate, self-assemble, and/or adhere to a cell. In some cases, a polymeric material may encapsulate, self-assemble, or adhere to a cell. In some cases, a polymeric material may encapsulate a cell. In some cases, a polymeric material may directly encapsulate a cell. In some cases, a polymeric material may also indirectly encapsulate a cell. In some cases, a polymeric material and a cell may self-assemble. In some cases, cell may adhere to a polymeric material.

In some instances, a nucleic acid molecule may alter the protein expression of a cell and may promote or facilitate differentiation of a cell. In some instances, a saccharide may promote or facilitate differentiation of a cell. In some instances, a cationic saccharide may promote or facilitate a differentiation of a cell. In some instances, a lipid may promote or facilitate a differentiation of a cell. In some instances, a nanoparticle may promote or facilitate a differentiation of a cell. In some instances, a chitosan may promote a differentiation of a cell.

The terms “promote,” “promotion,” “facilitate,” “facilitation,” or grammatically equivalent, when used herein referring to a cellular process (e.g., protein expression or differentiation), refers to lowering the threshold or increasing the occurrence of the cellular process. In some cases, if a molecular entity or a group of molecular entities facilitate(s) a cellular process of a cell, the cellular process has an increased probability to occur in the cell comprising the molecular entity or the group of molecular entities, relative to the cell not comprising the molecular entity or the group of molecular entities. For example, if a molecular entity or a group of molecular entities facilitate(s) a cellular process of a cell, the cellular process may be at least about 1%, 5%, 10%, 20%, 30%, 40%, 50%, 60%, 70%, 80%, 90%, 1 time, 1.5 times, 2 times, 2.5 times, 3 times, 4 times, 5 times, 6 times, 7 times, 8 times, 9 times, 10 times, 15 times, 20 times, 25 times, 30 times, 35 times, 40 times, 45 times, 50 times, 100 times, 1000 times or more likely to occur in the cell comprising the molecular entity or the group of molecular entities, relative to the cell not comprising the molecular entity or the group of molecular entities.

In some cases, if a molecular entity or a group of molecular entities facilitate(s) a cellular process of a group of cells, the cellular process has an increased probability to occur or an increased number of cells with the occurrence of the cellular process in the group of cells comprising the molecular entity or the group of molecular entities, relative to the group of cells not comprising the molecular entity or the group of molecular entities. For example, if a molecular entity or a group of molecular entities facilitate(s) a cellular process of a group cells, the cellular process may be at least about 1%, 5%, 10%, 20%, 30%, 40%, 50%, 60%, 70%, 80%, 90%, 1 time, 1.5 times, 2 times, 2.5 times, 3 times, 4 times, 5 times, 6 times, 7 times, 8 times, 9 times, 10 times, 15 times, 20 times, 25 times, 30 times, 35 times, 40 times, 45 times, 50 times, 100 times, 1000 times or more likely to occur in the group of cells comprising the molecular entity or the group of molecular entities, relative to the group of cells not comprising the molecular entity or the group of molecular entities. In other cases, if a molecular entity or a group of molecular entities facilitate(s) a cellular process of a group cells, the cellular process may occur in at least about 1%, 5%, 10%, 20%, 30%, 40%, 50%, 60%, 70%, 80%, 90%, 1 time, 1.5 times, 2 times, 2.5 times, 3 times, 4 times, 5 times, 6 times, 7 times, 8 times, 9 times, 10 times, 15 times, 20 times, 25 times, 30 times, 35 times, 40 times, 45 times, 50 times, 100 times, 1000 times or more cells in the group of cells comprising the molecular entity or the group of molecular entities, relative to the group of cells not comprising the molecular entity or the group of molecular entities.

The protein expression within a cell facilitated or promoted by a nucleic acid molecule may comprise a polypeptide or protein encoded by the nucleic acid molecule. In some cases, the nucleic acid molecule taken up by the cell may alter the expression of other proteins. For example, the nucleic acid molecule that contacts the cell may be translated by the cell into a polypeptide or protein. The polypeptide or protein may interact with other cellular components and facilitate or promote an alteration of expressions of other proteins, transcripts, nucleic acids, and/or other metabolites. In some cases, the nucleic acid molecule that contacts the cell may not be translated into a protein or polypeptide by the cell. The nucleic acid molecule, without itself being translated into a protein or polypeptide, may facilitate or promote expression of other cellular proteins. The nucleic acid molecule may affect interact with other cellular components and facilitate or promote an alteration of expressions of other proteins, transcripts, nucleic acids, and/or other metabolites.

In some cases, the facilitation or promotion of an alteration of expression of a protein within a cell by a nucleic acid molecule described herein may occur at the transcriptional level (e.g., an alteration of transcription of the gene encoding the protein), post-transcriptional level (e.g., an alteration of transcript stability, transport, RNA modification, or a combination thereof of the transcript encoding the protein), translational level (e.g., an alteration of translation of the transcript encoding the protein), post-translational level (e.g., an alteration of post-translational modifications of the proteins), or a combination thereof.

In some instances, promoting or facilitating an expression of a protein in a cell may comprise an increase in the expression level of the protein. The terms “cell(s)”, “cell population(s),” or “population(s) of cells,” or any grammatically equivalent terms may be used interchangeably. In some cases, the increase in the expression level of the protein may be at least about 1%, 5%, 10%, 20%, 30%, 40%, 50%, 60%, 70%, 80%, 90%, 1 time, 1.5 times, 2 times, 2.5 times, 3 times, 4 times, 5 times, 6 times, 7 times, 8 times, 9 times, 10 times, 15 times, 20 times, 25 times, 30 times, 35 times, 40 times, 45 times, 50 times, 100 times, 1000 times or more than the expression level of the protein without the increase. In some instances, promoting or facilitating an expression of a protein in a cell may comprise an increase in the number of cells in population of cells that may express the proteins. The increase in the number of cells that expressed the protein, with the aid of the compositions or methods of the present disclosure, may be at least about 1%, 5%, 10%, 20%, 30%, 40%, 50%, 60%, 70%, 80%, 90%, 1 time, 1.5 times, 2 times, 2.5 times, 3 times, 4 times, 5 times, 6 times, 7 times, 8 times, 9 times, 10 times, 15 times, 20 times, 25 times, 30 times, 35 times, 40 times, 45 times, 50 times greater than the number of cells that expressed the protein in the absence of the compositions or methods of the present disclosure. In some instances, promoting or facilitating an alteration of an expression of a protein in a cell may comprise a decrease in the expression level of the protein. In some cases, the decrease in the expression level of the protein may be at least about 1%, 5%, 10%, 20%, 30%, 40%, 50%, 60%, 70%, 80%, 90%, 95%, 99% or lower than the expression level of the protein without the decrease. In some instances, promoting or facilitating an expression of a protein in a cell may comprise an decrease in the number of cells in population of cells that may express the proteins. The decrease in the number of cells that expressed the protein, with the aid of the compositions or methods of the present disclosure, may be at least about 1%, 5%, 10%, 20%, 30%, 40%, 50%, 60%, 70%, 80%, 90%, 95%, 99% or fewer than the number of cells that expressed the protein in the absence of the compositions or methods of the present disclosure.

Cell that has contacted a composition provided herein comprising a nucleic acid molecule, or a progeny derive from the cell, may express at least 1%, 5%, 10%, 20%, 30%, 40%, 50%, 60%, 70%, 80%, 90%, 1 time, 1.5 times, 2 times, 2.5 times, 3 times, 4 times, 5 times, 6 times, 7 times, 8 times, 9 times, 10 times, 15 times, 20 times, 25 times, 30 times, 35 times, 40 times, 45 times, 50 times, 100 times, 1000 times or more the nucleic acid molecule, the transcript or derivative of the nucleic molecule, the protein or polypeptide encoded by the nucleic acid molecule, or a combination thereof, relative to the cell that has not contacted the composition or a progeny thereof. A population of cells that has contacted a composition provided herein comprising a nucleic acid molecule, or a progeny derive from the population of cells, may have at least 1%, 5%, 10%, 20%, 30%, 40%, 50%, 60%, 70%, 80%, 90%, 1 time, 1.5 times, 2 times, 2.5 times, 3 times, 4 times, 5 times, 6 times, 7 times, 8 times, 9 times, 10 times, 15 times, 20 times, 25 times, 30 times, 35 times, 40 times, 45 times, 50 times, 100 times, 1000 times or more cells that express the first nucleic acid molecule, the transcript or derivative of the second nucleic molecule, the protein or polypeptide encoded by the second nucleic acid molecule, or a combination thereof, relative to the population of cells that has not contacted the composition or a progeny thereof.

Cell that has contacted a composition provided herein comprising a first nucleic acid molecule, or a progeny derive from the cell, may express at least 1%, 5%, 10%, 20%, 30%, 40%, 50%, 60%, 70%, 80%, 90%, 1 time, 1.5 times, 2 times, 2.5 times, 3 times, 4 times, 5 times, 6 times, 7 times, 8 times, 9 times, 10 times, 15 times, 20 times, 25 times, 30 times, 35 times, 40 times, 45 times, 50 times, 100 times, 1000 times or more of a second nucleic acid molecule different from the first nucleic acid molecule, the transcript or derivative of the second nucleic molecule, the protein or polypeptide encoded by the second nucleic acid molecule, or a combination thereof, relative to the cell that has not contacted the composition or a progeny thereof. A population of cells that has contacted a composition provided herein comprising a first nucleic acid molecule, or a progeny derive from the population of cells, may have at least 1%, 5%, 10%, 20%, 30%, 40%, 50%, 60%, 70%, 80%, 90%, 1 time, 1.5 times, 2 times, 2.5 times, 3 times, 4 times, 5 times, 6 times, 7 times, 8 times, 9 times, 10 times, 15 times, 20 times, 25 times, 30 times, 35 times, 40 times, 45 times, 50 times, 100 times, 1000 times or more cells that express a second nucleic acid molecule different from the first nucleic acid molecule, the transcript or derivative of the second nucleic molecule, the protein or polypeptide encoded by the second nucleic acid molecule, or a combination thereof, relative to the population of cells that has not contacted the composition or a progeny thereof. Cell that has contacted a composition provided herein comprising a first nucleic acid molecule, or a progeny derive from the cell, may express at least 1%, 5%, 10%, 20%, 30%, 40%, 50%, 60%, 70%, 80%, 90%, 95%, 99% or less of a second nucleic acid molecule different from the first nucleic acid molecule, the transcript or derivative of the second nucleic molecule, the protein or polypeptide encoded by the second nucleic acid molecule, or a combination thereof, relative to the cell that has not contacted the composition or a progeny thereof. A population of cells that has contacted a composition provided herein comprising a first nucleic acid molecule, or a progeny derive from the population of cells, may have at least 1%, 5%, 10%, 20%, 30%, 40%, 50%, 60%, 70%, 80%, 90%, 95%, 99% or less cells that express a second nucleic acid molecule different from the first nucleic acid molecule, the transcript or derivative of the second nucleic molecule, the protein or polypeptide encoded by the second nucleic acid molecule, or a combination thereof, relative to the population of cells that has not contacted the composition or a progeny thereof.

Cell that has contacted a composition provided herein comprising a first nucleic acid molecule, or a progeny derive from the cell, may express at least 1%, 5%, 10%, 20%, 30%, 40%, 50%, 60%, 70%, 80%, 90%, 1 time, 1.5 times, 2 times, 2.5 times, 3 times, 4 times, 5 times, 6 times, 7 times, 8 times, 9 times, 10 times, 15 times, 20 times, 25 times, 30 times, 35 times, 40 times, 45 times, 50 times, 100 times, 1000 times or more of a second nucleic acid molecule different from the first nucleic acid molecule, the transcript or derivative of the second nucleic molecule, the protein or polypeptide encoded by the second nucleic acid molecule, or a combination thereof, relative to the cell that has only contacted the first nucleic acid molecule without the other components of the compositions described herein or a progeny thereof. A population of cells that has contacted a composition provided herein comprising a first nucleic acid molecule, or a progeny derive from the population of cells, may have at least 1%, 5%, 10%, 20%, 30%, 40%, 50%, 60%, 70%, 80%, 90%, 1 time, 1.5 times, 2 times, 2.5 times, 3 times, 4 times, 5 times, 6 times, 7 times, 8 times, 9 times, 10 times, 15 times, 20 times, 25 times, 30 times, 35 times, 40 times, 45 times, 50 times, 100 times, 1000 times or more cells that express a second nucleic acid molecule different from the first nucleic acid molecule, the transcript or derivative of the second nucleic molecule, the protein or polypeptide encoded by the second nucleic acid molecule, or a combination thereof, relative to the population of cells that has only contacted the first nucleic acid molecule without the other components of the compositions described herein or a progeny thereof.

Cell that has contacted a composition provided herein comprising a first nucleic acid molecule, or a progeny derive from the cell, may express at least 1%, 5%, 10%, 20%, 30%, 40%, 50%, 60%, 70%, 80%, 90%, 95%, 99% or less of a second nucleic acid molecule different from the first nucleic acid molecule, the transcript or derivative of the second nucleic molecule, the protein or polypeptide encoded by the second nucleic acid molecule, or a combination thereof, relative to the cell that has only contacted the first nucleic acid molecule without the other components of the compositions described herein or a progeny thereof. A population of cells that has contacted a composition provided herein comprising a first nucleic acid molecule, or a progeny derive from the population of cells, may have at least 1%, 5%, 10%, 20%, 30%, 40%, 50%, 60%, 70%, 80%, 90%, 95%, 99% or less cells that express a second nucleic acid molecule different from the first nucleic acid molecule, the transcript or derivative of the second nucleic molecule, the protein or polypeptide encoded by the second nucleic acid molecule, or a combination thereof, relative to the population of cells that has not only contacted the first nucleic acid molecule without the other components of the compositions described herein or a progeny thereof.

The alteration of protein expression may be measured at least about 1 hour, 2 hours, 3 hours, 4 hours, 5 hours, 6 hours, 7 hours, 8 hours, 9 hours, 10 hours, 11 hours, 12 hours, 13 hours, 14 hours, 15 hours, 16 hours, 17 hours, 18 hours, 19 hours, 20 hours, 21 hours, 22 hours, 23 hours, 24 hours, 25 hours, 26 hours, 27 hours, 28 hours, 29 hours, 30 hours, 31 hours, 32 hours, 33 hours, 34 hours, 35 hours, 36 hours, 37 hours, 38 hours, 39 hours, 40 hours, 41 hours, 42 hours, 43 hours, 44 hours, 45 hours, 46 hours, 47 hours, 48 hours, 3 days, 4 days, 5 days, 6 days, 7 days, 1 week, 2 weeks, 3 weeks, 4 weeks or more, subsequent to a cell is contacted with a nucleic acid, a saccharide, or a combination thereof, or a control thereof. The increase or decrease of protein expression may be measured at most about 1 hour, 2 hours, 3 hours, 4 hours, 5 hours, 6 hours, 7 hours, 8 hours, 9 hours, 10 hours, 11 hours, 12 hours, 13 hours, 14 hours, 15 hours, 16 hours, 17 hours, 18 hours, 19 hours, 20 hours, 21 hours, 22 hours, 23 hours, 24 hours, 25 hours, 26 hours, 27 hours, 28 hours, 29 hours, 30 hours, 31 hours, 32 hours, 33 hours, 34 hours, 35 hours, 36 hours, 37 hours, 38 hours, 39 hours, 40 hours, 41 hours, 42 hours, 43 hours, 44 hours, 45 hours, 46 hours, 47 hours, 48 hours, 3 days, 4 days, 5 days, 6 days, 7 days, 1 week, 2 weeks, 3 weeks, or 4 weeks, subsequent to a cell is contacted with a nucleic acid, a saccharide, or a combination thereof, or a control thereof.

In some cases, the alteration of protein expression of a cell may comprise a difference of protein expressed (or not express) or a difference of proteins expressed at a pre-determined level. Alteration of protein expression of a cell may comprise at least about 1, 5, 10, 50, 100, 500, 1000, 5000, 10000 or more proteins that are expressed or not expressed. Alteration of protein expression of a cell may comprise at least about 1, 5, 10, 50, 100, 500, 1000, 5000, 10000 or more proteins that are expressed or not expressed at a pre-determined level. The pre-determined level may be a level relative to a pre-determined control proteins. The control proteins may be the proteins expressed at a constant level by the cells in various cellular states or culture conditions. The control proteins may be the proteins expressed by house-keeping genes. The levels of a control protein may be within at most about 1%, 2%, 3%, 4%, 5%, 7%, 8%, 9%, 10%, 15%, 20%, 25%, or 30% when measured in the cells in two distinct cellular states or culture conditions. The expression level of a protein may be measured by quantitative real-time polymerase chain reaction, nucleic acid sequencing, fluorescent hybridization, or a combination thereof of the gene product encoding the protein (e.g., mRNA). The expression level of a protein may be measured by western blot, flow cytometry, mass-spectrometry, immunofluorescence, fluorescent tag/label, or a combination thereof of the protein.

The alteration of the expression of a protein described herein may be measured at the protein level. For example, western blot, eastern blot, immunofluorescence, antibody staining, mass spectrometry, gel electrophoresis, uses of a reporter that reflects the expression of the proteins, or any combination thereof may be used to measure the expression of the protein. The alteration of the expression of a protein described herein may be measured at the cell population level. For example, flow cytometry, counting of cells that express a protein (reporter or immunofluorescence), or a combination thereof may be used to measure the expression of the protein. The alteration of the expression of a protein described herein may be measured at the transcript level. For example, quantitative real-time PCR (QRT-PCR), reverse-transcription PCR, northern blot, RNA-sequencing, fluorescence hybridization, or a combination thereof may be used to measure the expression of the protein.

In some instances, promoting or facilitating differentiation of a cell may comprise an increase of the number of cells in population of cells that may undergo differentiation. The increase of the number of cells that undergoes differentiation, with the aid of the compositions or methods of the present disclosure, may be at least about 1%, 5%, 10%, 20%, 30%, 40%, 50%, 60%, 70%, 80%, 90%, 1 time, 1.5 times, 2 times, 2.5 times, 3 times, 4 times, 5 times, 6 times, 7 times, 8 times, 9 times, 10 times, 15 times, 20 times, 25 times, 30 times, 35 times, 40 times, 45 times, 50 times greater than the number of cells that undergoes differentiation in the absence of the compositions or methods of the present disclosure.

In some instances, a cell that has taken up a saccharide, a nucleic acid, a polyplex or a combination thereof may have a change in intracellular processes. In some cases, a change in intracellular process may comprise changes in expression (increase or decrease) of one or more proteins in the cell (e.g., with the aid of or due to the presence of the nucleic acid molecules). In some instances, two or more nucleic acid molecules may be used collectively to promote protein expression or facilitate differentiation of a cell. Each of the two or more nucleic acid molecules may be configured to affect an expression (e.g., enhancing or suppressing expression) of a given protein or gene in the cell. The two or more nucleic acid molecules may affect expression of the same protein or gene or different proteins or genes. The two or more nucleic acid molecules may collectively affect expression of the same protein, gene or different proteins or genes. When at least two nucleic molecules collectively facilitate protein expression or promote the differentiation of a cell, the overlap of the genes (or proteins or nucleic acids) with the expression affected by each nucleic acid molecule may comprise about 0%, 1%, 5%, 10%, 20%, 30%, 40%, 50%, 60%, 70%, 80%, 90%, 100% of the genes with altered gene expression.

In some instances, a nucleic acid and a saccharide are configured to collectively promote adipogenic differentiation of a cell. In some instances, a nucleic acid and a saccharide are configured to collectively promote angiogenic differentiation of a cell. In some instances, a nucleic acid and a saccharide are configured to collectively promote cardiogenic differentiation of a cell. In some instances, a nucleic acid and a saccharide are configured to collectively promote chondrogenic differentiation of a cell. In some instances, a nucleic acid and a saccharide are configured to collectively promote endothelial differentiation of a cell. In some instances, a nucleic acid and a saccharide are configured to collectively promote epithelial differentiation of a cell. In some instances, a nucleic acid and a saccharide are configured to collectively promote hematopoietic differentiation of a cell. In some instances, a nucleic acid and a saccharide are configured to collectively promote hepatogenic differentiation of a cell. In some instances, a nucleic acid and a saccharide are configured to collectively promote myogenic differentiation of a cell. In some instances, a nucleic acid and a saccharide are configured to collectively promote neurogenic differentiation of a cell. In some instances, a nucleic acid and a saccharide are configured to collectively promote osteogenic differentiation of a cell. In some instances, a nucleic acid and a saccharide are configured to collectively promote parenchymal differentiation of a cell. In some instances, a nucleic acid and a saccharide are configured to collectively promote renal differentiation of a cell. In some instances, a nucleic acid and a saccharide are configured to collectively promote retinal differentiation of a cell.

In some cases, a saccharide may facilitate or promote the differentiation of a cell by enhancing the uptake of a nucleic acid molecule by the cell. In some cases, a chitosan may facilitate or promote the differentiation of a cell by enhancing the uptake of a nucleic acid molecule by the cell. In some cases, a lipid may facilitate or promote the differentiation of a cell by enhancing the uptake of a nucleic acid molecule by the cell. In some cases, a nanoparticle may facilitate or promote the differentiation of a cell by enhancing the uptake of a nucleic acid molecule by the cell.

In some cases, a saccharide may facilitate or promote the differentiation of a cell by enhancing the uptake of an mRNA by the cell. In some cases, a chitosan may facilitate or promote the differentiation of a cell by enhancing the uptake of an mRNA by the cell. In some cases, a lipid may facilitate or promote the differentiation of a cell by enhancing the uptake of an mRNA by the cell. In some cases, a nanoparticle may facilitate or promote the differentiation of a cell by enhancing the uptake of an mRNA by the cell. In some cases, an mRNA may facilitate or promote the differentiation by enhancing the uptake of another nucleic acid molecule by the cell.

In some cases, a saccharide may facilitate or promote the differentiation of a cell by enhancing the uptake of a siRNA by the cell. In some cases, a chitosan may facilitate or promote the differentiation of a cell by enhancing the uptake of a siRNA by the cell. In some cases, a lipid may facilitate or promote the differentiation of a cell by enhancing the uptake of a siRNA by the cell. In some cases, a nanoparticle may facilitate or promote the differentiation of a cell by enhancing the uptake of a siRNA by the cell. In some cases, a siRNA may facilitate or promote the differentiation by enhancing the uptake of another nucleic acid molecule by the cell. In some cases, a saccharide may facilitate or promote the differentiation of a cell by enhancing the uptake of a miRNA by the cell. In some cases, a chitosan may facilitate or promote the differentiation of a cell by enhancing the uptake of a miRNA by the cell. In some cases, a lipid may facilitate or promote the differentiation of a cell by enhancing the uptake of a miRNA by the cell. In some cases, a nanoparticle may facilitate or promote the differentiation of a cell by enhancing the uptake of a miRNA by the cell. In some cases, a miRNA may facilitate or promote the differentiation by enhancing the uptake of another nucleic acid molecule by the cell.

In some cases, a nucleic acid molecule may facilitate or promote the differentiation by enhancing the uptake of another nucleic acid molecule by the cell. For example, a siRNA may facilitate or promote the differentiation by enhancing the uptake of an mRNA by the cell. In some cases, a miRNA may facilitate or promote the differentiation by enhancing the uptake of an mRNA by the cell. In some cases, a chitosan and a siRNA may facilitate or promote the differentiation by enhancing the uptake of an mRNA by the cell. In some cases, a chitosan and a miRNA may facilitate or promote the differentiation by enhancing the uptake of an mRNA by the cell.

In some cases, a polymeric material for encapsulating a cell may comprise a polymer. In some cases, a polymer may comprise a polysaccharide-based polymer, a polypeptide-based polymer, or a combination thereof. In some cases, a polymer may comprise a polysaccharide-based polymer. In some cases, a polymer may comprise a polypeptide-based polymer. In some cases, a polysaccharide-based polymer may comprise an alginate-based polymer, a gellan gum-based polymer, a cassava-based polymer, a maize-based polymer, a corn starch-based polymer, a xanthan gum-based polymer, a locust bean-based polymer, a pullulan-based polymer, a dextran-based polymer, a cellulose-based polymer, or a combination thereof. In some cases, a polysaccharide-based polymer may comprise an alginate-based polymer. In some cases, a polysaccharide-based polymer may comprise a gellan gum-based polymer. In some cases, a polysaccharide-based polymer may comprise a cassava-based polymer. In some cases, a polysaccharide-based polymer may comprise a maize-based polymer. In some cases, a polysaccharide-based polymer may comprise a corn starch-based polymer. In some cases, a polysaccharide-based polymer may comprise a xanthan gum-based polymer. In some cases, a polysaccharide-based polymer may comprise a locust bean-based polymer. In some cases, a polysaccharide-based polymer may comprise a pullulan-based polymer. In some cases, a polysaccharide-based polymer may comprise a dextran-based polymer. In some cases, a polysaccharide-based polymer may comprise a cellulose-based polymer. In some instances, a polypeptide-based polymer may comprise a plant protein-based polymer.

In some instances, a polymer may comprise a hydrogel. In some cases, hydrogel may comprise a substrate mediated strategy for delivering DNA complexes from hyaluronic acid (HA)-collagen hydrogels. In some cases, hydrogels are formed by crosslinking HA with poly(ethylene glycol) diglycidyl ether. In some cases, a topography of a hydrogel is introduced using pattern transfer. In some cases, nucleic acid molecules of various sizes are immobilized to the hydrogel substrate. In another instances, a polymer may comprise a collagen hydrogel. In some cases, a hydrogel either contains or can be chemically modified to contain a functional group allowing covalently bind to a bifunctional crosslinker or polylinker modified to have a functional group. In some cases, hydrogels may be synthetic polymers or biopolymer matrices that are highly hydrated (e.g. at least about 50%, by weight, of the hydrogel may comprise water). In some cases, a hydrogel may be structurally stable. A hydrogel may comprise polyacrylamides or poloxamers. Hydrogels may also comprise a naturally occurring polymer, such as collagen. In some cases, a hydrogel may comprise a freeze-dried collagen matrices.

A hydrogel may comprise alginate. An alginate-based hydrogel may form an insoluble aggregate in the presence of calcium. In some cases, a hydrogel precursor mixture may comprise a poly cation. In some cases, a polycation can, for example, be selected from the group consisting of polylysine, polyarginine, polyomithine, polyhistidine, myelin basic protein, a low molecular weight glycopeptide, a cationic amphiphilic alpha-helical oligopeptide having a repeating sequence, a histone, a galactosylated histone, polybrene, spermine, spermidine, prolamine, polyethylenimine, putrescine, cadaverine, and hexamine.

In some instances, a polymer may comprise a 2-dimensional polymer. In other cases, a polymer may comprise a 3-dimensional polymer. In some instance, a polymer is configured to be biodegradable. Among various polymers, polymers may form polyelectrolyte complexes with a nucleic acid and/or protect a nucleic acid molecule from various enzymes. In some cases, a polymer may comprise synthetic and natural polymer. In some cases, a polymer may comprise polyamine-based polymer. In some cases, a polymer may comprise cationic lipids, polyamine-based polymers, chitosan-based polymers, dendrimers, and polyethyleneimine (PEI). In some cases, a polymer may comprise a heparin-polyethyleneimine (HPEI) nanogel. In some cases, a polymer may can comprise a micelle prepared by grafting branched PEG on poly [(ε-caprolactone)-co-glycolide] (CG). In some cases, pullulan (a polysaccharide polymer consisting of maltotriose units) modified PEI may be biodegradable. In some cases, a polymer may comprise poly-β-aminoester (PBAE), a group of biodegradable polymers that comprises cationic amino groups and hydrolysable ester linkages. In some cases, a polymer may comprise polyamine (co-esters) that are produced by enzyme-catalyzed copolymerization of the lactone with diallyl diester and amino diols. In some cases, a polymer may comprise poly-L-Lysine (PLL), cationic peptide. In some cases, a polymer may comprise natural a biodegradable polymer. In some cases, a natural biodegradable polymer may comprise chitosan, pullulan, dextran, and hyaluronic acid (HA).

### Cells

In some cases, a cell type may comprise a cell that expresses a specific profile or group of genes, nucleic acid molecules, and/or proteins. In some cases, cells of a cell type may have a common expression profile or group of genes, nucleic acid molecules, and/or proteins. In some cases, cells of a cell type may express at least about 1, 2, 3, 4, 5, 6, 7, 8, 9, 10, 11, 12, 13, 14, 15, 16, 17, 18, 19, 20, 50, 100, 200, 300, 400, 500, 600, 700, 800, 900, 1000, 2000, 5000, 10000 or more identical genes, nucleic acid molecules, and/or proteins. In some cases, cells of a cell type may express at most about 1, 2, 3, 4, 5, 6, 7, 8, 9, 10, 11, 12, 13, 14, 15, 16, 17, 18, 19, 20, 50, 100, 200, 300, 400, 500, 600, 700, 800, 900, 1000, 2000, 5000, 10000, 20000, or 30000 identical genes, nucleic acid molecules, and/or proteins. Cells of a cell type may have shared structural or functional characteristics.

A cell may comprise cells or a cell population. A cell may comprise progenies of a cell or cell population. Cells may comprise progenies of a single cell.

In some instances, a cell may comprise a fat cell, a blood vessel cell, a cardiac cell, a chondrocyte, an endothelia cell, an epithelial cell, a hematopoietic cell, a hepatocyte, a muscle cell, a neuron, or an osteogenic cell. In some cases, a cell may comprise a fat cell. In some cases, a cell may comprise a blood vessel cell. In some cases, a cell may comprise a cardiac ell. In some cases, a cell may comprise a chondrocyte. In some cases, a cell may comprise an endothelia cell. In some cases, a cell may comprise an epithelial cell. In some cases, a cell may comprise a hematopoietic cell. In some cases, a cell may comprise a hepatocyte. In some cases, a cell may comprise a muscle cell. In some cases, a cell may comprise a neuron. In some cases, a cell may comprise or an osteogenic cell. In some instances, a cell may comprise at least 2, 3, 4, 5, 6, 7, 8, 9, 10, or 11 of a fat cell, a blood vessel cell, a cardiac ell, a chondrocyte, an endothelia cell, an epithelial cell, a hematopoietic cell, a hepatocyte, a muscle cell, a neuron, and an osteogenic cell. In some cases, a cementogenic cell comprises a cell that can form or be a part of a tooth. In some cases, an endothelia cell comprises a cell that can form or be a part of the endothelium. In some cases, an epithelial cell comprises a cell that can form or be a part of the epithelium. In some cases, a fat cell may comprise a cell that can form or be a part of the adipose tissues. In some cases, a hematopoietic cell may comprise a cell that can form or be a part of the blood or hematopoietic tissues. In some cases, a hepatocyte may comprise a cell that can form or be a part of the hepatic or liver tissue. In some cases, a muscle cell may comprise a cell that can form or be a part of the muscle tissues. In some cases, a neuron may comprise a cell that can form or be a part of the nervous systems. In some cases, a renal cell may comprise a cell that can form or be a part of the kidney tissues. In some cases, a retinal cell may comprise a cell that can form or be a part of the ocular or eye tissues. In some cases, an osteogenic cell may comprise a cell that can form or be a part of the bone tissues. In some cases, a blood vessel cell or an angiogenic cell may comprise a cell that can form or be a part of the blood vessel or vascular tissue. In some cases, a cardiac cell may comprise a cell that can form or be a part of the heart or cardiac tissue. In some cases, a chondrocyte cell may comprise a cell that can form or be a part of the cartilage tissue.

In some cases, a fat cell may be an adipocyte. In some cases, a fat cell may contain various sizes of fat droplets or granules. In some cases, a fat cell may comprise a white adipose cell or a brown adipose cell. In some cases, a white adipose cell may contain large fat droplets or granules and a small amount of cytoplasm. In some cases, a brown adipose cell may contain a large amount of cytoplasm and numerous mitochondria. In some cases, an adipocyte may be a cell primarily composed of adipose tissue, specialized in synthesizing and storing energy as fat. Adipocytes may be derived from induced pluripotent stem cells and/or mesenchymal stem cells through adipogenesis. Adipocytes may be white adipocytes, which store energy as a single large lipid droplet and have important endocrine functions, and brown adipocytes which store energy in multiple small lipid droplets but specifically for use as fuel to generate body heat.

In some cases, a muscle cell may develop sarcoplasm, sarcoplasmic reticulum, sarcosome, or sarcolemma that are specialized for muscle contraction and energy metabolism. In some cases, a muscle cell may contain myofibrils and myoglobins. In some cases, a muscle cell may contain a high amount of glycogen. In some cases, a muscle cell may also comprise a myocyte. In some cases, a muscle cell may develop from a myoblast. In some cases, a muscle cell may be a cardiac muscle cell, a smooth muscle cell, or a skeletal muscle cell. In some instances, a muscle cell may comprise a myofiber, a myotube, a myocyte, a myoblast, a spheroid, or a muscle cell progenitor.

In some instances, a cell may comprise an animal cell. An animal cell may comprise a cell isolated or derived from an organism from the kingdom Animalia. An animal cell may be isolated from an animal. A cell may also be an animal cell if the closest counterpart of its genome is from an animal or an animal cell.

In some instances, an animal cell may comprise a mammalian cell, a bird cell, or a fish cell, a mollusk cell, or an amphibian cell. In some instances, an animal cell may comprise a mollusk cell. In some instances, an animal cell may comprise an amphibian cell. In some instances, an animal cell may comprise a mollusk cell.

In some instances, an animal cell may comprise a mammalian cell. In some instances, an animal cell may comprise a bird cell. In some instances, an animal cell may comprise a fish cell.

In some instances, a mammalian cell may comprise a porcine cell, a bovine cell, a bubaline cell, an ovine cell, a caprine cell, a cervine cell, a bisontine cell, a cameline cell, an elaphine cell, or a lapine cell. In some cases, a cell may comprise a porcine cell. In some cases, a cell may comprise a bovine cell. In some cases, a cell may comprise a bubaline cell. In some cases, a cell may comprise an ovine cell. In some cases, a cell may comprise a caprine cell. In some cases, a cell may comprise a cervine cell. In some cases, a cell may comprise a bisontine cell. In some cases, a cell may comprise a cameline cell. In some cases, a cell may comprise an elaphine cell. In some cases, a cell may comprise a lapine cell.

In some instances, a bird cell may comprise an anatine cell, a galline cell, an anserine cell, a meleagrine cell, a struthionine cell, or a phasianine cell. In some cases, a cell may comprise an anatine cell. In some cases, a cell may comprise a galline cell. In some cases, a cell may comprise an anserine cell. In some cases, a cell may comprise a meleagrine cell. In some cases, a cell may comprise a struthionine cell. In some cases, a cell may comprise a phasianine cell.

In some instance, a differentiated cell may undergo self-renewal. In other cases, a differentiated cell may not undergo self-renewal. In some cases, a terminally differentiated cell may not undergo self-renewal. In some cases, a differentiated cell may not be at a pluripotent cell state.

In some cases, a cell that can undergo differentiation may comprise a stem cell. In other cases, a stem cell may be at a totipotent cell state. In some cases, a stem cell maybe at a pluripotent cell state. In other cases, a stem cell may be at a multipotent cell state. In other cases, a stem cell may be at an omnipotent cell state. In other cases, a stem cell may be at a unipotent cell state. In some cases, a stem cell may comprise an iPSC. In some cases, a stem cell may comprise an embryonic stem cell, an immortalized stem cell, a mesenchymal stem cell, and/or a muscle progenitor cell. In some cases, a stem cell may comprise an embryonic stem cell, an immortalized stem cell, a mesenchymal stem cell, an iPSC, and/or a muscle progenitor cell. In some cases, a stem cell may comprise an embryonic stem cell. In some cases, a stem cell may comprise an immortalized stem cell. In some cases, a stem cell may comprise a mesenchymal stem cell. In some cases, a stem cell may comprise a muscle progenitor cell.

A stem cell may comprise a reprogrammed cell. Cellular reprogramming may be a process that reverses the developmental potential of a cell or population of cells (e.g., a somatic cell). Reprogramming may be a process of driving a cell to a state with higher developmental potential, such as driving a cell backwards to a less differentiated state. The cell to be reprogrammed can be either partially or terminally differentiated prior to reprogramming. Reprogramming may infer a complete or partial reversion of the differentiation state, such as an increase in the developmental potential of a cell, to that of a cell having a pluripotent state, driving a somatic cell to a pluripotent state, such that the cell has the developmental potential of an embryonic stem cell, such as an embryonic stem cell phenotype, or may encompass a partial reversion of the differentiation state or a partial increase of the developmental potential of a cell, such as a somatic cell or a unipotent cell, to a multipotent state. Reprogramming may also encompass a partial reversion of the differentiation state of a cell to a state that renders the cell more susceptible to complete reprogramming to a pluripotent state when subjected to additional manipulations.

In some instances, a stem cell may comprise an animal stem cell. In some instances, an animal stem cell may comprise a mammalian stem cell, a bird stem cell, or a fish stem cell, a mollusk stem cell, or an amphibian stem cell. In some instances, an animal stem cell may comprise a mollusk stem cell. In some instances, an animal stem cell may comprise an amphibian stem cell. In some instances, an animal stem cell may comprise a mollusk stem cell. In some cases, a mammalian stem cell may comprise a porcine stem cell, a bovine stem cell, a bubaline stem cell, an ovine stem cell, a caprine stem cell, a cervine stem cell, a bisontine stem cell, a cameline stem cell, an elaphine stem cell, or a lapine stem cell. In some cases, a mammalian stem cell may comprise a porcine stem cell. In some cases, a bird stem cell may comprise an anatine stem cell, a galline stem cell, an anserine stem cell, a meleagrine stem cell, a struthionine stem cell, or a phasianine stem cell. In some instances, an animal stem cell may comprise a mammalian stem cell. In some instances, an animal stem cell may comprise a bird stem cell. In some instances, an animal stem cell may comprise a fish stem cell. In some cases, a stem cell may comprise a porcine stem cell. In some cases, a stem cell may comprise a bovine stem cell. In some cases, a stem cell may comprise a bubaline stem cell. In some cases, a stem cell may comprise an ovine stem cell. In some cases, a stem cell may comprise a caprine stem cell. In some cases, a stem cell may comprise a cervine stem cell. In some cases, a stem cell may comprise a bisontine stem cell. In some cases, a stem cell may comprise a cameline stem cell. In some cases, a stem cell may comprise an elaphine stem cell. In some cases, a stem cell may comprise a lapine stem cell. In some cases, a stem cell may comprise an anatine stem cell. In some cases, a stem cell may comprise a galline stem cell. In some cases, a stem cell may comprise an anserine stem cell. In some cases, a stem cell may comprise a meleagrine stem cell. In some cases, a stem cell may comprise a struthionine stem cell. In some cases, a stem cell may comprise a phasianine stem cell.

In some instances, an iPSC may comprise an animal iPSC. In some instances, an animal iPSC may comprise a mammalian iPSC, a bird iPSC, or a fish iPSC, a mollusk iPSC, or an amphibian iPSC. In some instances, an animal iPSC may comprise a mollusk iPSC. In some instances, an animal iPSC may comprise an amphibian iPSC. In some instances, an animal iPSC may comprise a mollusk iPSC. In some cases, a mammalian iPSC may comprise a porcine iPSC, a bovine iPSC, a bubaline iPSC, an ovine iPSC, a caprine iPSC, a cervine iPSC, a bisontine iPSC, a cameline iPSC, an elaphine iPSC, or a lapine iPSC. In some cases, a mammalian iPSC may comprise a porcine iPSC. In some cases, a bird iPSC may comprise an anatine iPSC, a galline iPSC, an anserine iPSC, a meleagrine iPSC, a struthionine iPSC, or a phasianine iPSC. In some instances, an animal iPSC may comprise a mammalian iPSC. In some instances, an animal iPSC may comprise a bird iPSC. In some instances, an animal iPSC may comprise a fish iPSC. In some cases, an iPSC may comprise a porcine iPSC. In some cases, an iPSC may comprise a bovine iPSC. In some cases, an iPSC may comprise a bubaline iPSC. In some cases, an iPSC may comprise an ovine iPSC. In some cases, an iPSC may comprise a caprine iPSC. In some cases, an iPSC may comprise a cervine iPSC. In some cases, an iPSC may comprise a bisontine iPSC. In some cases, an iPSC may comprise a cameline iPSC. In some cases, an iPSC may comprise an elaphine iPSC. In some cases, an iPSC may comprise a lapine iPSC. In some cases, an iPSC may comprise an anatine iPSC. In some cases, an iPSC may comprise a galline iPSC. In some cases, an iPSC may comprise an anserine iPSC. In some cases, an iPSC may comprise a meleagrine iPSC. In some cases, an iPSC may comprise a struthionine iPSC. In some cases, an iPSC may comprise a phasianine iPSC.

In some cases, iPSCs may comprise any cells obtained by re-programming of adult somatic cells which are endowed with pluripotency, a cell being capable of differentiating into the three embryonic germ cell layers, the endoderm, ectoderm and mesoderm. Such adult cells may be obtained from any adult somatic tissue (e.g. a skin fibroblast or blood cells) and undergo reprogramming by integrative genetic manipulation or non-integrative protein expression methods, which reset the cell to acquire stem cell-like characteristics. iPSCs may be formed through such processes that reverses the development of the cell or population of cells (e.g., a somatic cell) thus resulting in a naive cell type. An iPSC may be a cell that has undergone a process of driving a cell to a naive state with higher developmental and proliferation potential, such as a cell that is reset into a less differentiated state. The somatic cell, prior to induction to an iPSC, can be either partially or terminally differentiated. There may be a complete or partial reversion of the differentiation state, i.e., an increase in the developmental potential of a cell, to that of a cell having a pluripotent state. A somatic cell may be driven to a pluripotent state, such that the cell has the developmental potential of an embryonic stem cell, similar to an embryonic stem cell phenotype. Induction of a somatic cell may also encompass a partial reversion of the differentiation state or a partial increase of the developmental potential of a cell, such as a somatic cell or a unipotent cell, to a multipotent state. Induction may also encompass partial reversion of the differentiation state of a cell to a state that renders the cell more susceptible to complete induction to a pluripotent state when subjected to additional manipulations.

### Therapeutic or Edible Products

In some instances, a cell that has contacted the composition or has taken up the nucleic acid molecule may be cultured. Some or all of the cells thereof may subsequently be cultured to generate cultured cells, which cultured cells may be differentiated to generate terminally differentiated cells. In some cases, the cell comprising the nucleic acid molecules may be used to produce a therapeutic product. In some cases, the cell comprising the nucleic acid molecules may be used to produce a tissue. In some cases, the cell comprising the nucleic acid molecules may be used to produce an edible meat product. The terminally differentiated cells can be used to produce an edible meat product.

In some cases, a therapeutic product may have a therapeutic effect when administered. A therapeutic effect may comprise an inhibition, amelioration, mitigation, treatment, and/or prevention of a disease condition. In some cases, a therapeutic product may comprise a cell used in cell therapy. In some cases, a therapeutic product may comprise a cell, a vaccine, an organ, a product produced by the cell. The product produced by the cell may comprise a protein, a nucleic acid molecule, a chemical compound, or a combination thereof. In some cases, a protein may comprise an antibody, an enzyme, a signaling molecule, an enzyme inhibitor, a hormone, a cytokine, a growth factor or a combination thereof. A nucleic acid molecule used as a therapeutic product may comprise any nucleic molecules described elsewhere in this disclosure. In some cases, a chemical compound may comprise an organic or inorganic compound. In some cases, a compound may comprise a nutrient. In some cases, a cell used as a therapeutic product may comprise any cell described elsewhere in this disclosure. In some cases, the therapeutic product may comprise a human or a non-human cell, protein, nucleic acid molecule, chemical compound, or a combination thereof. In some cases, a product produced by the cell may comprise an antimicrobial molecule. The antimicrobial molecule may be an antibacterial, an antifungal, or an antiparasitic molecule.

A therapeutic product may comprise a medicine. A medicine may comprise a chemical or a collection and/or mixture of chemicals that has a therapeutic effect when administered. In some cases, a therapeutic product may comprise a drug product. In some cases, a drug product may be a therapeutic product in a dosage form. In some cases, a therapeutic product may comprise a pharmaceutically active ingredient. A pharmaceutically active ingredient may comprise a chemical and/or cell that has a direct pharmaceutical activity contributes to and/or is responsible for the inhibition, amelioration, mitigation, treatment, and/or prevention of a disease condition. In some cases, a therapeutic product may comprise a drug substance. In some cases, a drug substance may comprise a pure form of the pharmaceutically active ingredient. In some cases, a sample comprising a drug substance may comprise at least 50%, 60%, 70%, 80%, 90%, or 100%, by volume, weight, and/or number of molecules, of the drug substance in the sample.

A tissue may comprise a collection of cells. The cells of a tissue may be from one cell type. The cells of a tissue may be from more than one cell type. In some cases, a tissue or group of cells may form an organ. In some cases, the group of tissues or cells of an organ may be found in an animal. In some cases, the tissues or cells from an organ may collectively perform a physiological or cellular function. In some cases, the tissues or cells from an organ may share structural and/or functional characteristics. In some cases, an organ may comprise a bladder, a blood vessel, a bone, a brain, a bronchi, a cartilage, a diaphragm, a fallopian tube, a gill, a hair, a heart, a hypothalamus, an intestine, a kidney, a larynx, a ligament, a liver, a lung, a lymph node, a muscle, a nail, a nerve, an ovary, a pancreas, a parathyroid, a penis, a pharynx, a pineal body, a pituitary gland, a prostate, a scale, a skin, a spinal cord, a spleen, a stomach, a tendon, a testis, a thymus, a thyroid, a tonsil, a tooth, a trachea, a ureters, a urethra, a vagina, a vas deferen, a vulva, or a combination thereof. In some cases, a tissue or group of cells may form an organoid. In some cases, the organoid may be formed in an in vitro or ex vivo culture. In some cases, the organoid may be 3D. In some cases, the tissues or cells from an organoid may collectively perform a physiological or cellular function. In some cases, the tissues or cells from an organoid may share structural and/or functional characteristics. In some cases, a model may comprise a cell or a collection of cells. In some cases, a model comprising a cell or a collection of cells may be a model of the organ, the organoid, the tissue, or a combination thereof. In some cases, a model comprising a cell or a collection of cells may be a model of the organ. In some cases, a model comprising a cell or a collection of cells may be a model of the organoid. In some cases, a model comprising a cell or a collection of cells may be a model of the tissue. In some cases, a model may have at least one functional and/or structural characteristic of the cell, the collection of cells, the tissue, the organ, the organoid, or a combination thereof. In some cases, a model may comprise organic or inorganic materials. In some cases, a model may comprise a cell, tissue, organ, or organoid; or a component derived thereof. In some cases, a model may be a biomimetic model.

An edible product may also comprise a group of cells. An edible product may also comprise a group of cells having a population of cell types of a tissue from an animal. An edible product may be edible when it is consumed by any species, and it causes no harm. Such harm may comprise poisoning, contamination, or infection. An edible product may comprise an edible meat product. The edibility of a meat may depend on the types or properties of the meat. For example, the meats may be processed or cooked to be edible. In some cases, meats may be baked, steamed, poached, boiled, grilled, dried, smoked, fried, heated, pickled, fermented, aged, or any combination thereof. Some meats, such as those from fish, may be edible without cooking. The edibility of a meat may depend on the level of toxins and contaminating organisms. In some cases, the edibility of a meat may also depend on the appetite of a person.

In some cases, cultured cells may receive some degrees of structural integrity from a scaffold on which the cells may be attached during culturing. A cell may also be cultured in cell suspensions. In some cases, a cell may be adherent. In other cases, a cell may not be adherent. In some cases, non-adherent cells may or may not require a substrate or surface for attachment. In some cases, cells may have been selected, evolved, modified or engineered to not require an adherence substrate. In some cases, cultured cells may be grown or configured to form cultured tissues that may be attached to a support structure such as a two-dimensional (2D) or three-dimensional (3D) scaffold or support structure. In some cases, cultured cells may be configured to form various shapes or forms by a three-dimension printer. In some cases, cultured cells may be grown on a two-dimensional support structure such as a tissue-culture plate where they may form several layers of cells that may be peeled and processed for consumption. In some cases, two-dimensional support structures may include porous membranes that allow for diffusion of nutrients from culture media on one side of the membrane to the other side where the cells are attached. In such a composition, additional layers of cells may be achieved provided media perfusion is sufficient e.g. by exposing the cells to culture media from both sides of the membrane. In some cases, cells may receive nutrients through diffusion from one side of the membrane and also from the culture media covering the cells growing on the membrane. Culture media may be replenished in the two-dimensional tissue culture environment to prevent the buildup of waste metabolites.

In some cases, cultured cells may be grown on, around, or inside a three-dimensional support structure. In some cases, the support structure may be sculpted into different sizes, shapes, and/or forms to provide the shape and form for the cultured cells to grow and resemble different types of tissues such as steak, tenderloin, shank, chicken breast, drumstick, lamb chops, fish fillet, or lobster tail. The support structure may be a natural or synthetic biomaterial. In some cases, a biomaterial may comprise any substance intended to interface with biological systems to evaluate, treat, augment, or replace any tissue, organ, or function in a biocompatible manner, such as with a level of acceptable biological response. In some cases, a biomaterial may interact passively with cells and tissues or may comprise a bioactive material which induces a specific and intended biological response. In some cases, a biomaterial may comprise a substrate that has been engineered to take a form which alone or as part of a complex system, is used to direct, by control of interactions with components of living systems. In some cases, a biomaterial may be natural, synthetic, or some combination thereof. A scaffold may be composed of one material or one or more different materials. In some cases, a scaffold may be non-toxic and edible so that they may not be harmful if ingested and may provide additional nutrition, texture, flavor, or form to the final food product. In some cases, a scaffold may comprise a hydrogel, a biomaterial such as an extracellular matrix molecule (ECM), cellulose, or biocompatible synthetic materials. ECM molecules may comprise proteoglycans, non-proteoglycan polysaccharides, or proteins. In some cases, a micro-scaffold may be smaller than a traditional tissue culture scaffold which may provide a macroscopic structure and/or shape for the cell population. In some cases, a micro-scaffold may provide a surface for adherent cells to attach to even while the micro-scaffold itself is in suspension. In some cases, a micro-scaffold may provide a seed or core structure for adherent cells to attach while remaining small enough to remain in suspension with stirring. The use of micro-scaffolds enables the culturing of adherent cells in a suspension culture which may enable the large-scale production of adherent cells. In some cases, cultured cells may be allowed to grow in aggregates, spheroids or embryoid bodies. ECM molecules will form between the cells providing a naturally occurring tissue culture scaffold to which cells continue to grow and adhere to.

A degradable scaffold may comprise a polymeric material. A polymeric material may comprise a natural polymeric material or a synthetic polymeric material. Biomaterials may comprise collagen, gelatin, fibrin, alginate, agar, cassava, maize, chitosan, gellan gum, corn-starch, chitin, cellulose, chia (Salvia hispanica), recombinant silk, decellularized tissue (plant or animal), hyaluronic acid, fibronectin, laminin, hemicellulose, glucomannan, textured vegetable protein, heparan sulfate, chondroitin sulfate, tempeh, keratan sulfate, or any combination thereof. A plant-based scaffold may be used for 3D culturing. A plant-based scaffold may comprise scaffolds obtained from plants such as apples, seaweed, or jackfruit. A plant-based scaffold may comprise at least about one plant-based material such as cellulose, hemicellulose, pectin, lignin, alginate, or any combination thereof. A textured vegetable protein (TVP), such as textured soy protein (TSP) may comprise a high percentage of soy protein, soy flour, or soy concentrate. TVP and TSP can be used to provide a meat-like texture and consistency to a meat product. Synthetic biomaterials may comprise hydroxyapatite, polyethylene terephthalate, acrylates, polyethylene glycol, polyglycolic acid, polycaprolactone, polylactic acid, their copolymers, or any combination thereof.

In some instances, a method may comprise expanding or culturing a cell. In some instances, an expanding or a culturing may comprise maintenance media, differentiation media, steatotic media, or proliferation media. In some cases, a media may be configured to promote cell culturing or expansion. In some cases, gas balance in the media may comprise a mixture of oxygen from about 21% to about 95% air saturation, Carbon dioxide partial pressure (mm Hg) from about 0% to about 10%. For example, a mixture of media of about 80% Oxygen about 5% carbon dioxide and held at 37° C. with controlled pH may be provided. In some cases, complete transfection media are equilibrated overnight at 37° C. and 5% CO₂ and pH adjustment is performed with. In certain cases, terminal differentiation may comprise the sequential steps of culturing or maintaining a plurality of substantially undifferentiated pluripotent cells in a first defined media comprising at least about one growth factor, and incubating the cells in a second defined media which is sufficient to promote differentiation in a plurality of cells. In some aspects, a plurality of the pluripotent cells is differentiated into endodermal, ectodermal or mesodermal cells HPCs. In certain aspects, the second defined media may comprise FGF2, IGF-1. In certain aspects, the second defined media may comprise a GSK-3 inhibitor. In some cases, the first defined media further may comprise IGF-1, NRG-1, TGF-B, LIF and FGF2. In some cases, the second defined media further may comprise FGF2. In some cases, the method may comprise culturing the cells at an atmospheric pressure of less than 25% oxygen, such as less than 24%, 23%, 22%, 21%, 20%, 19%, 18%, 17%, 16%, 15%, 10%, 5%, or 0% oxygen.

In some cases, a maintenance media may comprise pluripotency media. In some cases, cells are treated with high dose (50 ng/mL) porcine-FGF2 for 48 hours prior to transfection. In some instances, a media is changed to second defined media after 72 hours. In some instances, at least about a portion of an expanding or a culturing is performed using a growth mode other than simple batch culture. As used herein for culturing cells, culturing a cell in vitro may also comprise culturing a cell, a group of cells, or a tissue ex vivo or outside of an organism or a host. A cell culture, in some cases, may also comprise the maintenance or induction of the differentiation (or de-differentiation) of a cell. In other cases, a cell culture may be maintained in growth media. A cell culture, in some cases, may be 2-dimensional (2D) or 3-dimensional (3D). In some cases, growth media may comprise nutrients or other components required for the growth of a cell. In some cases, the types of media and the nutrients for a cell culture may depend on the cell being cultured or the purpose of the cultured cell.

### Examples

These examples are provided for illustrative purposes only and not to limit the scope of the claims provided herein.

### Example 1. Transfection of RNAs into Cells with Saccharides and Nucleic Acid Molecules

Provided here are various saccharide, nucleic acid molecule, lipid, and polyplex combinations for transfecting cells with a composition comprising nucleic acid molecules and saccharides.

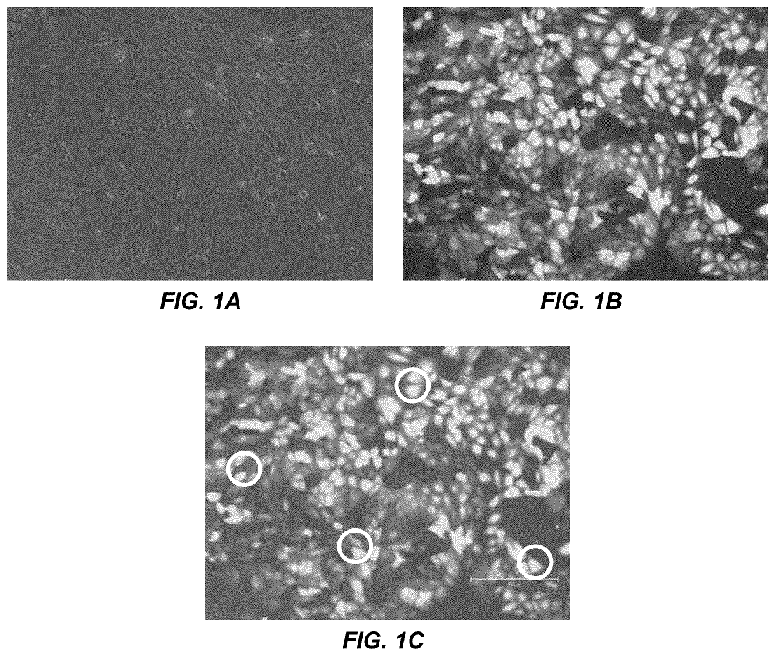

A polyplex of e-GFP mRNA (nucleic acid molecule; TriLink Biotechnologies, Product #L-7601) with a cationized starch (saccharide) and lipid was formed by mixing the aliquots of stock solutions of individual components in PBS pH 7.2 in 1/2/4 nucleic acid molecule/saccharide/lipid mass ratio. The polyplex was allowed to form for 20 minutes at room temperature and used to treat porcine iPSCs grown in 24-well plate (seeding density 15000 cells per well) for 24 hours. mRNA dosage 0.5 μg per well. Plates were incubated at 37° C. with 5% atmospheric CO₂ for 20 hours. FIG. 1A, FIG. 1B, and FIG. 1C show the transmitted light image, the green fluorescence, the combination image of the porcine iPSC treated with eGFP-mRNA polyplex based on the cationized starch, respectively. As shown from FIGS. 1A-C, a majority of the cells were transfected with and expressed eGFP.

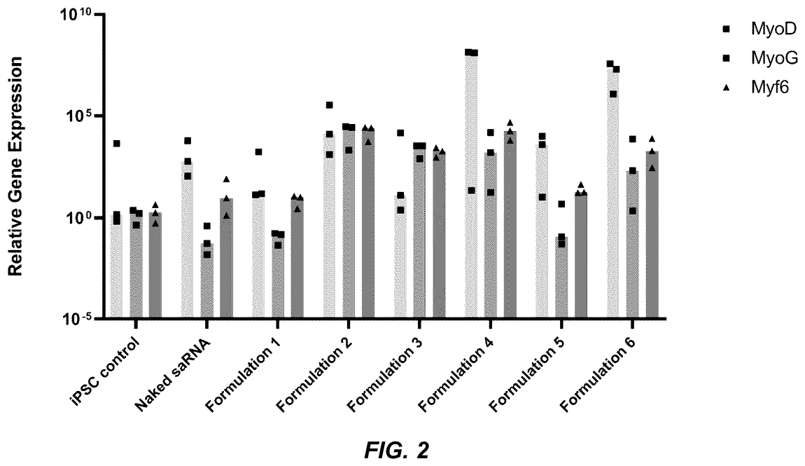

A double functionalized chitosan was prepared by introducing quaternary ammonium groups and conjugating with maltodextrin. A polyplex of saRNA (nucleic acid molecule; with sequences encoding MyoD, MyoG, and Myf6) with a double functionalized chitosan (saccharide) and lipid was formed by mixing the aliquots of stock solutions of individual components in PBS pH 7.2 in 1/300-600/300 nucleic acid molecule/saccharide/lipid mass ratio. The polyplex was allowed to form for 20 minutes at room temperature and used to treat porcine iPSCs grown in 24-well plate (seeding density 15000 cells per well) for 24 hours. saRNA dosage 0.2 ng per well. Plates were incubated at 37° C., 5% atmospheric CO₂ for 20 hours. FIG. 2 shows the gene expression in porcine iPSC transfected with saRNA delivered by polyplex based on double-functionalized chitosan. Formulation 1 is a transfection composition without lipid; formulations 2-4 are transfection complexes with increasing amounts of saccharides; formulation 5 is a negative control transfection complex in which the lipid was replaced with anionic polysaccharide; formulation 6 is a positive control with transfection complex derived from JetMESSENGER® (Polyplus). Other negative controls include iPSCs not contacting the saRNAs or the cells contacted with the saRNAs but not the saccharide or lipid. Expression of the three genes were assayed with QRT-PCR 24 hours after the transfection. Inclusion of saccharides and lipids significantly increases the expression of the transfected genes by about 10000-fold to 10000000-fold, relative to the negative controls using naked saRNA or untreated cells. Compared to transfection using the JetMESSENGER® system, improvement of transfection efficiency using the methods descried herein using saccharides and lipids is about 2 to 50 fold (FIG. 2).

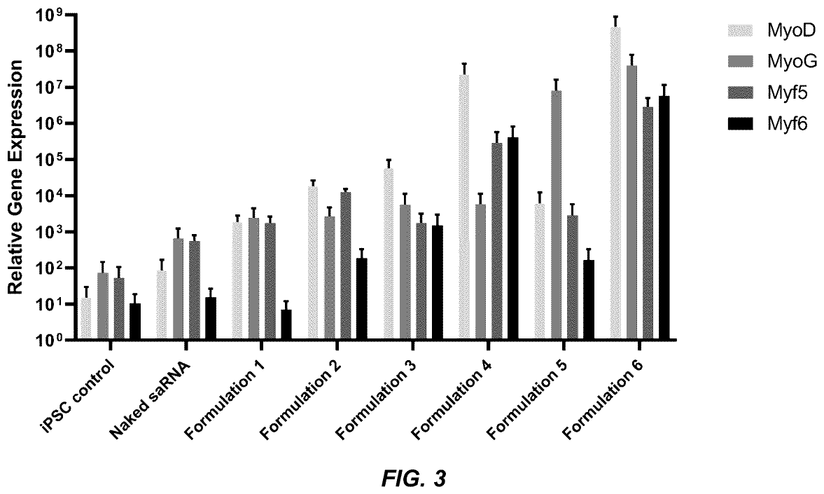

A polyplex of saRNA (nucleic acid molecule; with sequences encoding MyoD, MyoG, Myf5, and Myf6) with a cationized starch and lipid was formed by mixing the aliquots of stock solutions of individual components in PBS pH 7.2 in mass ratio 1/300-600/300 nucleic acid molecule/saccharide/lipid mass ratio. The polyplex was allowed to form for 20 minutes at room temperature and used to treat porcine iPSCs grown in 24-well plate (seeding density 15000 cells per well) for 24 hours. mRNA dosage 0.2 ng per well. Plates were incubated at 37° C. with 5% atmospheric CO₂ for 20 hours. FIG. 3 shows the gene expression in porcine iPSC transfected with saRNA delivered by polyplex based on cationized starch. Formulation 1 is a transfection composition without lipid; formulations 2-4 are transfection complexes with increasing amounts of saccharides; formulation 5 is a negative control transfection complex in which the lipid was replaced with anionic polysaccharide; formulation 6 is a positive control with transfection complex derived from JetMESSENGER® (Polyplus). Other negative controls include iPSCs not contacting the saRNAs or the cells contacted with the saRNAs but not the saccharide or lipid. Expression of the three genes were assayed with QRT-PCR 24 hours after the transfection. Inclusion of saccharides and lipids significantly increases the expression of the transfected genes by about 100-fold to 1000-fold, relative to the negative controls using naked saRNA or untreated cells. Transfection efficiency using the methods descried herein using saccharides and lipids is compared to that using the JetMESSENGER® system (FIG. 3).

The cationized starch or double functionalized chitosan can be substituted with other saccharides described in this disclosure. The saRNA or mRNA can be substituted with other nucleic acid molecules described in this disclosure.

### Example 2. Induction of Protein Expression, Cell Differentiation, or Cell Conversion Using Saccharides and Nucleic Acid Molecules

Provided herein are methods for facilitating protein expression, cell differentiation, and/cell conversion using the compositions/methods described in this disclosure.

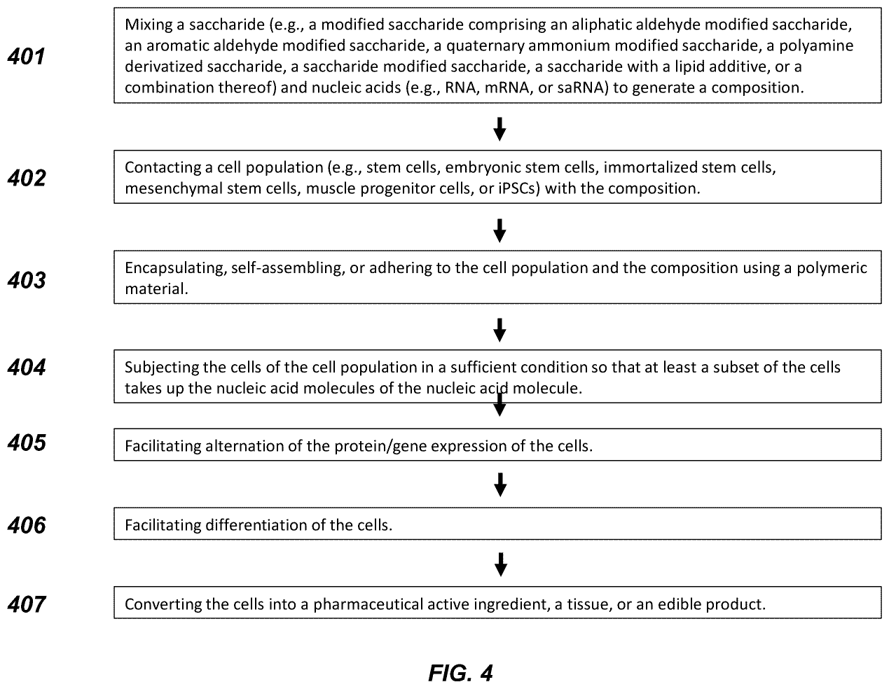

FIG. 4 depicts a workflow of the method described herein. Step 401 comprises mixing a saccharide (e.g., a modified saccharide comprising an aliphatic aldehyde modified saccharide, an aromatic aldehyde modified saccharide, a quaternary ammonium modified saccharide, a polyamine derivatized saccharide, a saccharide modified saccharide, a saccharide with a lipid additive, or a combination thereof) and nucleic acids (e.g., RNA, mRNA, or saRNA) to generate a composition. Step 402 comprises contacting a cell population (e.g., stem cells, embryonic stem cells, immortalized stem cells, mesenchymal stem cells, muscle progenitor cells, or iPSCs) with the composition. Step 403 comprises encapsulating, self-assembling, or adhering to the cell population and the composition using a polymeric material. Step 404 comprises subjecting the cells of the cell population in a sufficient condition so that at least a subset of the cells takes up the nucleic acid molecules of the nucleic acid molecule. Step 405 comprises facilitating the transfected cells to alter their protein expression. Optionally, step 406 comprises facilitating the differentiation of the transfected cells. Optionally, step 407 comprises converting the transfected cells into a pharmaceutical active ingredient, a tissue, or an edible product.

A few milligrams of modified saccharide, 2 mg of thiamine pyrophosphate, and 0.5 mg of protamine sulfate are independently dissolved in 1 mL of sterile distilled water. Each solution is filtered through a 0.22 μm filter. Targeting nucleic acid is dissolved to a final concentration of 1 mg/mL in sterile distilled water. PEG-lipid is dissolved in 1 mL of ethanol with the total PEG-lipid weight amounting to 30 mg, followed by filtration through a 0.22 μm filter. The nucleic acid solution is mixed with the protamine solution to form pre-complexes and then mixed with the water-soluble saccharide solution, the thiamine pyrophosphate solution, and the PEG-lipid solution to prepare compositions containing polyplexes.

Cells (listed in Table 1) are transfected daily with various saccharides and nucleic acid molecules (listed in Table 2 and 3, respectively) between 1-7 days. GFP/RFP/YFP mRNA, or scrambled siRNA are used as a transfection control. Transfection is carried out using either traditional chemical based methods (e.g. Lipofection), or non-chemical methods (e.g. electroporation or nucleofection) as controls for the colloidal nanoparticle method of choice. The diverse nature of nucleotides affects the delivery method chosen as can be seen in the difference of nucleotide lengths, double vs single stranded nucleic acids, and the dose range of nucleotides: Silencing RNA (siRNA): 20-40 bps, double-stranded RNA molecule, messenger RNA (mRNA): range of 500 bp-2-4 kbp, single stranded RNA molecule. Dose range of nucleotides: 0.5 μg/mL-50 μg/mL per nucleotide (DNA, RNA, mRNA, siRNA, saRNA). For example, mRNA and siRNA may be encapsulated together using a nanoparticle transfection option.

TABLE 1
Example cell types

| No | Cell type |
|---|---|
| 1 | fat cell |
| 2 | blood vessel cell |
| 3 | cardiac ell |
| 4 | chondrocyte cell |
| 5 | endothelia cell |
| 6 | epithelial cell |
| 7 | hematopoietic cell |
| 8 | hepatocyte cell |
| 9 | muscle cell |
| 10 | neuron cell |
| 11 | osteogenic cell |

TABLE 2
Examples of modified saccharides

| No | Modified Saccharides | |
|---|---|---|
| 1 | oligomeric saccharide | |
| 2 | polymeric saccharide | |
| 3 | aliphatic aldehyde | hexanal aldehyde modified saccharide |
| 4 | modified saccharide | heptanal aldehyde modified saccharide |
| 5 | | octanal aldehyde modified saccharide |
| 6 | | nonanal aldehyde modified saccharide |
| 7 | | decanal aldehyde modified saccharide |
| 8 | aromatic aldehyde | benzaldehyde modified saccharide |
| 9 | modified saccharide | cinnamaldehyde modified saccharide |
| 10 | polyamine derivatized | spermine modified saccharide |
| 11 | saccharide | spermidine modified saccharide |
| 12 | | putrescine modified saccharide |
| 13 | | diethylethylamine modified saccharide |
| 14 | | dimethylethylamine modified saccharide |
| 15 | | quaternary ammonium modified saccharide |
| 16 | | arginine modified saccharide |
| 14 | saccharide with a | saccharide with lecithin or saccharide |
| | lipid additive | with phosphatidylcholine |
| 15 | saccharide modified | polysaccharide modified |
| | saccharide | saccharide/monosaccharide |
| | | modified saccharide |
| 16 | | maltodextrin modified saccharide |

TABLE 3
Examples of nucleic acids

| No | Nucleic Acids |
|---|---|
| 1 | DNA |
| 2 | messenger ribonucleic acid (mRNA) |
| 3 | micro ribonucleic acid (miRNA) |
| 4 | transfer ribonucleic acid (tRNA) |
| 5 | silencing ribonucleic acid (siRNA) |
| 6 | self-amplifying RNA (saRNA) |

### Example 3. Culturing an Edible Meat Product

Cells are maintained and expanded in the first growth media supplemented with the required growth factors for the cell line in question. Cells are grown either on 2D adherent surface or in 3D as aggregates. To promote protein expression, cells are treated for 24 hours with the polyplex containing the nucleic acid self-assembled with the chosen delivery agent. Transfections are carried out in 2D (with or without biomaterial) or 3D (including but not limited to: spheroid, embryoid bodies, suspension or adherent, with or without biomaterial) culture conditions. Media may be changed after 24 hours. Cells are maintained in the second growth media for 7-50+ (min-max or anywhere in between) days depending on the desired outcome required. Maturation of cultures are carried out in the described 2D or 3D conditions, with or without biomaterial, or with or without electrical stimulation or contractile tension forces e.g. to promote maturation of myogenic fibers. The necessary controls are carried out for experimental consistency. Analysis may be conducted at any stage using qPCR, immunohistochemistry or flow cytometry. Any nucleic acid may be used in permutations to this methodology. Experimental changes may use the same materials and methods, but different compounds may be introduced. Following the required growth of cells (e.g. muscle cells expressing relevant myogenic markers, fat cells expressing relevant adipogenic markers), the cells are harvested and enter into food processing. Briefly, the cells are blended with plant based ingredients to the required quantities dependent on the end product in question. Between 1-99% (min-max) cell mass (or anywhere in between) may be used in the final 100% formulation. The formulation is formed into the desired shape and structure, sliced, cooked and frozen.

### NUMBERED EMBODIMENTS

1. A composition comprising: a saccharide and a nucleic acid molecule, which said nucleic acid molecule is configured to facilitate a change in protein expression within a cell; and a polymeric material that is configured to encapsulate or adhere to said cell.

2. The composition of embodiment 1, wherein said polymeric material comprises a polymer.

3. The composition of embodiment 2, wherein said polymer comprises a polysaccharide-based polymer, a polypeptide-based polymer, a lipid-based polymer, or a combination thereof.

4. The composition of embodiment 3, wherein said polysaccharide-based polymer comprises an alginate-based polymer, a gellan gum-based polymer, a cassava-based polymer, a maize-based polymer, a corn starch-based polymer, a xanthan gum-based polymer, a locust bean-based polymer, a pullulan-based polymer, a dextran-based polymer, a cellulose-based polymer, or a combination thereof.

5. The composition of any one of embodiments 1-4, wherein said polymeric material is configured to encapsulate said cell.

6. The composition of any one of embodiments 1-5, wherein said polymeric material comprises a hydrogel.

7. The composition of embodiment 6, wherein said polymeric material comprises a 2-dimensional polymer.

8. The composition of embodiment 6, wherein said polymer comprises a 3-dimensional polymer.

9. The composition of any one of embodiments 1-8, wherein said polymeric material is configured to be biodegradable.

10. A composition comprising: a nucleic acid molecule comprising a ribonucleic acid (RNA) and a saccharide which are configured to collectively facilitate a change in protein expression within a cell.

11. The composition of embodiment 10, wherein said change in protein expression within said cell facilitates differentiation of said cell into a mesodermal lineage, an endodermal lineage, or an ectodermal lineage.

12. The composition of embodiment 11, wherein said change in protein expression within said cell facilitates adipogenic, angiogenic, cardiogenic, chondrogenic, endothelial, epithelial, hematopoietic, hepatogenic, myogenic, neurogenic, osteogenic, parenchymal, renal, or retinal differentiation of said cell.

13. The composition of embodiment 12, wherein said differentiation of said cell comprises transdifferentiation of said cell or directed differentiation of said cell.

14. The composition of any one of embodiments 10-13, wherein said cell comprises a somatic cell or a naive cell.

15. The composition of any one of embodiments 1-14, wherein said cell comprises a fat cell, a blood vessel cell, a cardiac cell, a chondrocyte, an endothelial cell, an epithelial cell, a hematopoietic cell, a hepatocyte, a muscle cell, a neuron, or an osteogenic cell.

16. The composition of embodiment 15, wherein said cell comprises said muscle cell.

17. The composition of embodiment 16, wherein said muscle cell comprises a myofiber, a myotube, a myocyte, a myoblast, a myogenic spheroid, or a muscle cell progenitor.

18. A composition comprising: a saccharide and a nucleic acid molecule which are configured to collectively facilitate a change in protein expression within a cell, wherein said cell is a stem cell comprising an embryonic stem cell, an immortalized stem cell, a mesenchymal stem cell, a muscle progenitor cell, or an induced pluripotent stem cell (iPSC).

19. The composition of embodiment 18, wherein said cell comprises said iPSC.

20. The composition of any one of embodiments 1-9 and 18-19, wherein said nucleic acid molecule comprises a ribonucleic acid (RNA).

21. The composition of any one of embodiments 10-17 and 20, wherein said RNA comprises a messenger ribonucleic acid (mRNA), a micro ribonucleic acid (miRNA), a transfer ribonucleic acid (tRNA), a silencing ribonucleic acid (siRNA), a self-amplifying RNA (saRNA), an endless/circular RNA (eRNA), a trans-amplifying RNA (ta-RNA), or a combination thereof.

22. The composition of embodiment 21, wherein said RNA comprises said mRNA, said saRNA, said eRNA, said ta-RNA, or a combination thereof.

23. The composition of embodiment 22 or embodiment 23, wherein said RNA encodes MYOD1, MYOG, MYF5, MYF6, PAX3, PAX7, PPARY, adiponectin, FATP1-6, FABP4, GLUT4, Leptin, AdipoR1-2, CD137, a fragment thereof, or a variant thereof.

24. The composition of any one of embodiments 21-23 wherein said RNA is monocistronic.

25. The composition of any one of embodiments 21-23, wherein said RNA is polycistronic.

26. The composition of any one of embodiments 21-25, wherein said nucleic acid molecule comprises said mRNA.

27. The composition of any one of embodiments 21-25, wherein said nucleic acid molecule comprises said saRNA

28. The composition of any one of embodiments 21-27, wherein said nucleic acid molecule comprises said miRNA or said siRNA.

29. The composition of embodiment 28, wherein said miRNA or said siRNA comprises a polynucleotide sequence that facilitates a reduction of pluripotency of said cell.

30. The composition of embodiment 29, wherein said reduction of pluripotency of said cell facilitates said differentiation of said cell.

31. The composition of embodiment 29 or embodiment 30, wherein said polynucleotide sequence comprises a polynucleotide sequence that comprises POUF51 (OCT3/4), KLF4, or SOX2, or a complementary sequence thereof.

32. The composition of any one of embodiments 21-31, wherein said nucleic acid molecule comprises a first nucleic acid molecule and a second nucleic molecule, wherein said first nucleic acid molecule is said mRNA or said saRNA, and wherein said second nucleic acid is said miRNA or said siRNA.

33. A composition comprising a nucleic acid molecule and a modified saccharide, wherein said nucleic acid molecule comprises a messenger ribonucleic acid (mRNA), a self-amplifying RNA (saRNA), an endless/circular RNA (eRNA), a trans-amplifying RNA (ta-RNA), or a combination thereof, wherein said modified saccharide comprises an aliphatic aldehyde modified saccharide, an aromatic aldehyde modified saccharide, a quaternary ammonium modified saccharide, a polyamine derivatized saccharide, a saccharide modified saccharide, a saccharide with a lipid additive, or a combination thereof; and wherein said nucleic acid or said modified saccharide is configured to facilitate a change in protein expression within a cell.

34. The composition of embodiment 33, wherein said modified saccharide comprises a chitosan, a hyaluronic acid, a pullulan, a heparin, an alginate, or a combination or derivative thereof.

35. The composition of embodiment 34, wherein said modified saccharide comprises a modified chitosan.

36. The composition of any one of embodiments 33-35, wherein said modified saccharide comprises an aliphatic aldehyde modified saccharide, an aromatic aldehyde modified saccharide, a quaternary ammonium modified saccharide, a polyamine derivatized saccharide, a saccharide modified saccharide, saccharide a phenol modified saccharide, or a combination thereof.

37. The composition of any one of embodiments 33-36, wherein said aliphatic aldehyde modified saccharide comprises a hexanal aldehyde modified saccharide, a heptanal aldehyde modified saccharide, an octanal aldehyde modified saccharide, a nonanal aldehyde modified saccharide, or a decanal aldehyde modified saccharide.

38. The composition of embodiment 36 or embodiment 37, wherein said aliphatic aldehyde modified saccharide comprises a decanal aldehyde modified saccharide.

39. The composition of any one of embodiments 33-38, wherein said aromatic aldehyde modified saccharide comprises a benzaldehyde modified saccharide or a cinnamaldehyde modified saccharide.

40. The composition of any one of embodiments 33-39, wherein said polyamine derivatized saccharide comprises a spermine modified saccharide, a spermidine modified saccharide, a putrescine modified saccharide, diethylethylamine modified saccharide, dimethylethylamine modified saccharide, quaternary ammonium modified saccharide, or an arginine modified saccharide.

41. The composition of any one of embodiments 33-40, wherein said saccharide with said lipid additive comprises a saccharide with a lecithin or a saccharide with a phosphatidylcholine.

42. The composition of any one of embodiments 33-41, wherein said saccharide modified saccharide comprises a monosaccharide modified saccharide.

43. The composition of embodiment 42, wherein said monosaccharide modified saccharide comprises a lactose modified saccharide, a mannose modified saccharide, a glucose modified saccharide, a galactose modified saccharide, a glucosamine modified saccharide, a sucrose modified saccharide, a xylose modified saccharide, a ribose modified saccharide, a fructose modified saccharide, or a glyceraldehyde modified saccharide.

44. The composition of any one of embodiments 33-41, wherein said saccharide modified saccharide comprises a polysaccharide modified saccharide.

45. The composition of embodiment 44 wherein said polysaccharide modified saccharide comprises an anionic polysaccharide modified saccharide, a maltose modified saccharide, a reducing polysaccharide modified saccharide, or a combination thereof.

46. The composition of embodiment 45, wherein said anionic polysaccharide modified saccharide comprises an alginate modified saccharide, a carboxymethylated cellulose modified saccharide, a hyaluronic acid modified saccharide, a pectin modified saccharide, a pullulan modified saccharide, a starch modified saccharide, or a xanthan gum modified saccharide.

47. The composition of embodiment 45 or embodiment 46, wherein said reducing polysaccharide modified saccharide comprises a maltodextrin modified saccharide or a cellobiose modified saccharide.

48. The composition of any one of embodiments 45-47, wherein said reducing polysaccharide modified saccharide comprises a cationic lipid additive.

49. The composition of any one of embodiments 36-48, wherein said phenol modified saccharide comprises a chlorogenic acid modified saccharide, a ferulic acid modified saccharide, a caffeic acid modified saccharide, a gallic acid modified saccharide, or a combination thereof.

50. The composition of any one of embodiments 33-49, wherein said nucleic acid molecule encodes MYOD1, MYOG, MYF5, MYF6, PAX3, PAX7, PPARY, adiponectin, FATP1-6, FABP4, GLUT4, Leptin, AdipoR1-2, CD137, a fragment thereof, or a variant thereof.

51. The composition of any one of embodiments 33-50, wherein said nucleic acid molecule is monocistronic.

52. The composition of any one of embodiments 33-50, wherein said nucleic acid molecule is polycistronic.

53. The composition of any one of embodiments 33-52, further comprising a micro-ribonucleic acid (miRNA) or a silencing ribonucleic acid (siRNA).

54. The composition of embodiment 53, wherein said miRNA or said siRNA comprises a polynucleotide sequence that facilitates a reduction of pluripotency of said cell.

55. The composition of embodiment 54, wherein said reduction in pluripotency of said cell facilitates said differentiation of said cell.

56. The composition of embodiment 55, wherein said polynucleotide sequence comprises a polynucleotide sequence that comprises POUF51 (OCT3/4), KLF4, or SOX2, or a complementary sequence thereof.

57. The composition of any one of embodiments 53-56, wherein said nucleic acid molecule comprises a first nucleic acid molecule and a second nucleic molecule, wherein said first nucleic acid molecule is said mRNA or said saRNA, and wherein said second nucleic acid is said miRNA or said siRNA.

58. The composition of any one of embodiments 1-57, wherein said change in said protein expression within said cell facilitates differentiation of said cell.

59. The composition of any one of embodiments 1-58, further comprising a lipid, an anionic polymer, or a combination thereof.

60. The composition of any one of embodiments 1-59, wherein said saccharide is cationic in an aqueous solution or in a neutral solution.

61. The composition of any one of embodiments 1-60, wherein a cationic charge of cationic moieties of said saccharide comprises at least about 0.5 mequivalent of basic group per gram of said saccharide (mequiv/g).

62. The composition of any one of embodiments 1-61, wherein said cationic charge of said cationic moieties of said saccharide comprises at most about 20 mequiv/g.

63. The composition of any one of embodiments 1-62, wherein said saccharide comprises an average molecular mass of at least about 20 kilodaltons (kDa).

64. The composition of any one of embodiments 1-63, wherein said saccharide comprises an average molecular mass of at most about 2000 kDa.

65. The composition of any one of embodiments 1-64, wherein said saccharide comprises a polysaccharide.

66. The composition of any one of embodiments 1-65, wherein said saccharide and said nucleic acid molecule are configured to self-assemble with each other.

67. The composition of any one of embodiments 1-66, wherein said saccharide and said nucleic acid molecule are configured to form a polyplex.

68. The composition of embodiment 67, wherein said nucleic acid molecule is at or near a surface of said polyplex.

69. The composition of embodiment 67, wherein said nucleic acid molecule is encapsulated within said polyplex.

70. The composition of any one of embodiments 67-69, wherein said polyplex is regularly shaped, irregularly shaped, or branched.

71. The composition of any one of embodiments 67-69, wherein said polyplex is spherical or linear.

72. The composition of any one of embodiments 67-71, wherein an apparent diameter of said polyplex is at least about 5 nanometer (nm), as measured by dynamic light scattering (DLS) or microscopy.

73. The composition of any one of embodiments 67-72, wherein an apparent diameter of said polyplex is at most about 5000 nm, as measured by said DLS or said microscopy.

74. The composition of any one of embodiments 67-73, wherein said polyplex further comprises a nanoparticle.

75. The composition of any one of embodiments 1-74, wherein said nucleic acid molecule comprises an RNA-regulatory element.

76. The composition of embodiment 75, wherein said RNA-regulatory element comprises a transcriptional regulatory element, a co-transcriptional regulatory element, a post-transcriptional regulatory element, a translational regulatory element, or any combination thereof.

77. The composition of embodiment 75, wherein said RNA-regulatory element comprises a 5′-cap, 5′-untranslated region (UTR), a 3′-UTR, a poly-A tail modification, or any combination thereof.

78. The composition of any one of embodiments 1-77, wherein said saccharide stabilizes said nucleic acid molecule.

79. The composition of any one of embodiments 1-78, wherein said saccharide inhibits or reduces degradation of said nucleic acid molecule.

80. The composition of any one of embodiments 1-79, wherein said saccharide inhibits or reduces nuclease degradation of said nucleic acid molecule.

81. The composition of any one of embodiments 1-80, wherein said nucleic acid molecule comprises a chemical modification.

82. The composition of any one of embodiments 1-81, wherein said nucleic acid molecule comprises an unlocked nucleic acid.

83. The composition of any one of embodiments 1-82, wherein said nucleic acid molecule comprises at least two types of nucleic acids.

84. The composition of any one of embodiments 1-83, wherein said cell comprises a mammalian cell, a bird cell, a fish cell, a mollusks cell, or an amphibian cell.

85. The composition of embodiment 84, wherein said mammalian cell comprises a porcine cell, a bovine cell, a bubaline cell, an ovine cell, a caprine cell, a cervine cell, a bisontine cell, a cameline cell, an elaphine cell, or a lapine cell.

86. The composition of embodiment 85, wherein said mammalian cell comprises said porcine cell.

87. The composition of any one of embodiments 1-86, wherein a molar ratio of polycation amino groups of said polyplex to nucleic acid phosphate groups of said polyplex is at least about 1.

88. The composition of any one of embodiments 1-87, wherein a molar ratio of polycation amino groups of said polyplex to nucleic acid phosphate groups of said polyplex is at most about 60.

89. The composition of any one of embodiments 59-88, wherein the composition comprises said nucleic acid, said polymeric material, and said lipid, wherein a first mass ratio of said polymeric material to said nucleic acid is no less than 1:1, and wherein a second mass ratio of said lipid to said polymeric material is no less than 1:1.

90. The composition of embodiment 89, wherein said first mass ratio is no less than 2:1; and wherein said second mass ratio is no less than 2:1.

91. The composition of embodiment 90, wherein said first mass ratio is no less than 100:1.

92. A method for facilitating a change in protein expression within a cell, said method comprising:
(a) contacting a cell with a composition comprising:
   i. a saccharide, and
   ii. a nucleic acid molecule;
(b) encapsulating or adhering said cell and said composition using a polymeric material,
   thereby facilitating a change in protein expression within said cell.

93. The method of embodiment 92, wherein said polymeric material comprises a polymer.

94. The method of embodiment 93, wherein said polymer comprises a polysaccharide-based polymer, a polypeptide-based polymer, a lipid-based polymer, or a combination thereof.

95. The method of embodiment 94, wherein said polysaccharide-based polymer comprises an alginate-based polymer, a gellan gum-based polymer, a cassava-based polymer, a maize-based polymer, a corn starch-based polymer, a xanthan gum-based polymer, a locust bean-based polymer, a pullulan-based polymer, a dextran-based polymer, a cellulose-based polymer, or a combination thereof.

96. The method of any one of embodiments 92-95, wherein said polymeric material is configured to encapsulate said cell.

97. The method of any one of embodiments 92-96, wherein said polymeric material comprises a hydrogel.

98. The method of embodiment 97, wherein said polymeric material comprises a 2-dimensional polymer.

99. The method of embodiment 98, wherein said polymer comprises a 3-dimensional polymer.

100. The method of any one of embodiments 92-99, wherein said polymeric material is configured to be biodegradable.

101. A method for facilitating a change in protein expression within a cell, said method comprising:
contacting said cell with a composition comprising:
   i. a saccharide, and
   ii. a nucleic acid molecule comprising a ribonucleic acid (RNA);
      under conditions sufficient for said cell to uptake said composition, thereby facilitating said change in protein expression within said cell.

102. The method of embodiment 101, wherein said change in protein expression within said cell facilitates differentiation of said cell into a mesodermal lineage, an endodermal lineage, or an ectodermal lineage.

103. The method of embodiment 102, wherein said change in protein expression within said cell facilitates adipogenic, angiogenic, cardiogenic, chondrogenic, endothelial, epithelial, hematopoietic, hepatogenic, myogenic, neurogenic, osteogenic, parenchymal, renal, or retinal differentiation of said cell.

104. The method of any one of embodiments 101-103, wherein said differentiation of said cell comprises transdifferentiation of said cell or directed differentiation of said cell.

105. The method of any one of embodiments 101-104, wherein said cell comprises a somatic cell or a naive cell.

106. The method of any one of embodiments 101-105, wherein said cell comprises a fat cell, a blood vessel cell, a cardiac cell, a chondrocyte, an endothelial cell, an epithelial cell, a hematopoietic cell, a hepatocyte, a muscle cell, a neuron, or an osteogenic cell.

107. The method of embodiment 106, wherein said cell comprises said muscle cell.

108. The method of embodiment 107, wherein said muscle cell comprises a myofiber, a myotube, a myocyte, a myoblast, a myogenic spheroid, or a muscle cell progenitor.

109. A method for facilitating a change in protein expression within a cell, said method comprising:
contacting said cell with a composition comprising:
   i. a saccharide, and
   ii. a nucleic acid molecule;
      under conditions sufficient for said stem to uptake said composition, wherein said cell comprises a stem cell comprising an embryonic stem cell, an immortalized stem cell, a mesenchymal stem cell, a muscle progenitor cell, or an induced pluripotent stem cell (iPSC), thereby facilitating said change in said protein expression in said stem cell.

110. The method of embodiment 109, wherein said cell comprises said iPSC.

111. The method of any one of embodiments 92-100 and 109-110, wherein said nucleic acid molecule comprises an ribonucleic acid (RNA).

112. The method of any one of embodiments 101-108 and 111, wherein said RNA comprises a messenger ribonucleic acid (mRNA), a micro ribonucleic acid (miRNA), a transfer ribonucleic acid (tRNA), a silencing ribonucleic acid (siRNA), a self-amplifying RNA (saRNA), an endless/circular RNA (eRNA), a trans-amplifying RNA (ta-RNA), or a combination thereof.

113. The method of embodiment 112, wherein said RNA comprises said mRNA, said saRNA, said eRNA, said ta-RNA, or a combination thereof.

114. The method of embodiment 112 or embodiment 113, wherein said RNA encodes MYOD1, MYOG, MYF5, MYF6, PAX3, PAX7, PPARY, adiponectin, FATP1-6, FABP4, GLUT4, Leptin, AdipoR1-2, CD137, a fragment thereof, or a variant thereof.

115. The method of embodiment 112, wherein said RNA is monocistronic.

116. The method of embodiment 112, wherein said RNA is polycistronic.

117. The method of any one of embodiments 112-116, wherein said nucleic acid molecule comprises said mRNA.

118. The method of any one of embodiments 112-116, wherein said nucleic acid molecule comprises said saRNA

119. The method of any one of embodiments 112-118, wherein said nucleic acid molecule comprises said miRNA or said siRNA.

120. The method of embodiment 119, wherein said miRNA or said siRNA comprises a polynucleotide sequence that facilitates a reduction of pluripotency of said cell.

121. The method of embodiment 120, wherein said reduction in pluripotency of said cell facilitates said differentiation of said cell.

122. The method of embodiment 120 or embodiment 121, wherein said polynucleotide sequence comprises a polynucleotide sequence that comprises POUF51 (OCT3/4), KLF4, or SOX2, or a complementary sequence thereof.

123. The method of any one of embodiments 112-122, wherein said nucleic acid molecule comprises a first nucleic acid molecule and a second nucleic molecule, wherein said first nucleic acid molecule is said mRNA or said saRNA, and wherein said second nucleic acid is said miRNA or said siRNA.

124. A method for facilitating a change in protein expression within a cell, said method comprising:
contacting said cell with a composition comprising:
   i. a modified saccharide comprising an aliphatic aldehyde modified saccharide, an aromatic aldehyde modified saccharide, a quaternary ammonium modified saccharide, a polyamine derivatized saccharide, a saccharide modified saccharide, a saccharide with a lipid additive, or a combination thereof, and
   ii. a nucleic acid molecule comprising a messenger ribonucleic acid (mRNA), a self-amplifying RNA (saRNA), an endless/circular RNA (eRNA), a trans-amplifying RNA (ta-RNA), and a combination thereof;
under conditions sufficient for said cell to uptake said composition, thereby facilitating said change in protein expression within said cell.

125. The method of embodiment 124, wherein said modified saccharide comprises a chitosan, a hyaluronic acid, a pullulan, a heparin, an alginate, or a combination or derivative thereof.

126. The method of embodiment 125, wherein said modified saccharide comprises a modified chitosan.

127. The method of any one of embodiments 124-126, wherein said modified saccharide comprises an aliphatic aldehyde modified saccharide, an aromatic aldehyde modified saccharide, a quaternary ammonium modified saccharide, a polyamine derivatized saccharide, a saccharide modified saccharide, saccharide a phenol modified saccharide, or a combination thereof.

128. The method of any one of embodiments 124-127, wherein said aliphatic aldehyde modified saccharide comprises a hexanal aldehyde modified saccharide, a heptanal aldehyde modified saccharide, an octanal aldehyde modified saccharide, a nonanal aldehyde modified saccharide, or a decanal aldehyde modified saccharide.

129. The method of embodiment 127 or embodiment 128, wherein said aliphatic aldehyde modified saccharide comprises a decanal aldehyde modified saccharide.

130. The method of any one of embodiments 124-129, wherein said aromatic aldehyde modified saccharide comprises a benzaldehyde modified saccharide or a cinnamaldehyde modified saccharide.

131. The method of any one of embodiments 124-130, wherein said polyamine derivatized saccharide comprises a spermine modified saccharide, a spermidine modified saccharide, a putrescine modified saccharide, diethylethylamine modified saccharide, dimethylethylamine modified saccharide, quaternary ammonium modified saccharide, or an arginine modified saccharide.

132. The method of any one of embodiments 124-131, wherein said saccharide with said lipid additive comprises a saccharide with a lecithin or a saccharide with a phosphatidylcholine.

133. The method of any one of embodiments 124-132, wherein said saccharide modified saccharide comprises a monosaccharide modified saccharide.

134. The method of embodiment 133, wherein said monosaccharide modified saccharide comprises a lactose modified saccharide, a mannose modified saccharide, a glucose modified saccharide, a galactose modified saccharide, a glucosamine modified saccharide, a sucrose modified saccharide, a xylose modified saccharide, a ribose modified saccharide, a fructose modified saccharide, or a glyceraldehyde modified saccharide.

135. The method of any one of embodiments 124-134, wherein said saccharide modified saccharide comprises a polysaccharide modified saccharide.

136. The method of embodiment 135 wherein said polysaccharide modified saccharide comprises an anionic polysaccharide modified saccharide, a maltose modified saccharide, a reducing polysaccharide modified saccharide, or a combination thereof.

137. The method of embodiment 136, wherein said anionic polysaccharide modified saccharide comprises an alginate modified saccharide, a carboxymethylated cellulose modified saccharide, a hyaluronic acid modified saccharide, a pectin modified saccharide, a pullulan modified saccharide, a starch modified saccharide, or a xanthan gum modified saccharide.

138. The method of embodiment 136 or embodiment 137, wherein said reducing polysaccharide modified saccharide comprises a maltodextrin modified saccharide or a cellobiose modified saccharide.

139. The method of any one of embodiments 136-138, wherein said reducing polysaccharide modified saccharide comprises a cationic lipid additive.

140. The method of any one of embodiments 127-139, wherein said phenol modified saccharide comprises a chlorogenic acid modified saccharide, a ferulic acid modified saccharide, a caffeic acid modified saccharide, a gallic acid modified saccharide, or a combination thereof.

141. The method of any one of embodiments 124-140, wherein said nucleic acid molecule encodes MYOD1, MYOG, MYF5, MYF6, PAX3, PAX7, PPARY, adiponectin, FATP1-6, FABP4, GLUT4, Leptin, AdipoR1-2, CD137, a fragment thereof, or a variant thereof.

142. The method of any one of embodiments 124-141, wherein said nucleic acid molecule is monocistronic.

143. The method of any one of embodiments 124-141, wherein said nucleic acid molecule is polycistronic. 144. The method of any one of embodiments 124-143, the composition further comprises a micro-ribonucleic acid (miRNA) or a silencing ribonucleic acid (siRNA).

145. The method of embodiment 144, wherein said miRNA or said siRNA comprises a polynucleotide sequence that facilitates a reduction of pluripotency of said cell.

146. The method of embodiment 145, wherein said reduction in pluripotency of said cell facilitates said differentiation of said cell.

147. The method of embodiment 146, wherein said polynucleotide sequence comprises a polynucleotide sequence that comprises POUF51 (OCT3/4), KLF4, or SOX2, or a complementary sequence thereof.

148. The method of any one of embodiments 144-147, wherein said nucleic acid molecule comprises a first nucleic acid molecule and a second nucleic molecule, wherein said first nucleic acid molecule is said mRNA or said saRNA, and wherein said second nucleic acid is said miRNA or said siRNA.

149. The method of any one of embodiments 92-148, wherein said change in said protein expression within said cell facilitates differentiation of said cell.

150. The method of any one of embodiments 92-149, wherein said composition further comprises a lipid, an anionic polymer, or a combination thereof.

151. The method of any one of embodiments 92-150, wherein said saccharide is configured to be cationic in an aqueous solution or in a neutral solution.

152. The method of any one of embodiments 92-151, wherein a cationic charge of cationic moieties of said saccharide comprises at least about 0.5 mequivalent of basic group per gram of said saccharide (mequiv/g).

153. The method of any one of embodiments 92-152, wherein said cationic charge of said cationic moieties of said saccharide comprises at most about 20 mequiv/g.

154. The method of any one of embodiments 92-153, wherein said saccharide comprises an average molecular mass of at least about 20 kilodaltons (kDa).

155. The method of any one of embodiments 92-154, wherein said saccharide comprises an average molecular mass of at most about 2000 kDa.

156. The method of any one of embodiments 92-155, wherein said saccharide comprises a polysaccharide.

157. The method of any one of embodiments 92-156, wherein said saccharide and said nucleic acid molecule are configured to self-assemble with each other.

158. The method of any one of embodiments 92-157, wherein said saccharide and said nucleic acid molecule are configured to form a polyplex.

159. The method of embodiment 158, wherein said nucleic acid molecule is at or near a surface of said polyplex.

160. The method of embodiment 158, wherein said nucleic acid molecule is encapsulated within said polyplex.

161. The method of any one of embodiments 158-160, wherein said polyplex is regularly shaped, irregularly shaped, or branched.

162. The method of any one of embodiments 158-160, wherein said polyplex is spherical or linear.

163. The method of any one of embodiments 158-162, wherein an apparent diameter of said polyplex is at least about 5 nanometer (nm), as measured by dynamic light scattering (DLS) or microscopy.

164. The method of any one of embodiments 158-163, wherein an apparent diameter of said polyplex is at most about 5000 nm, as measured by said DLS or said microscopy.

165. The method of any one of embodiments 158-164, wherein said polyplex further comprises a nanoparticle.

166. The method of any one of embodiments 92-165, wherein said nucleic acid molecule comprises an RNA-regulatory element.

167. The method of embodiment 166, wherein said RNA-regulatory element comprises a transcriptional regulatory element, a co-transcriptional regulatory element, a post-transcriptional regulatory element, a translational regulatory element, or any combination thereof.

168. The method of embodiment 166, wherein said RNA-regulatory element comprises a 5′-cap, 5′-untranslated region (UTR), a 3′-UTR, a poly-A tail modification, or any combination thereof.

169. The method of any one of embodiments 92-168, wherein said saccharide is configured to stabilize said nucleic acid molecule.

170. The method of any one of embodiments 92-169, wherein said saccharide is configured to inhibit or reduce degradation of said nucleic acid molecule.

171. The method of any one of embodiments 92-170, wherein said saccharide is configured to inhibit a or reduce nuclease degradation of said nucleic acid molecule.

172. The method of any one of embodiments 92-171, wherein said nucleic acid molecule comprises a chemical modification.

173. The method of any one of embodiments 92-172, wherein said nucleic acid molecule comprises an unlocked nucleic acid.

174. The method of any one of embodiments 92-173, wherein said nucleic acid molecule comprises at least two types of nucleic acids.

175. The method of any one of embodiments 92-174, wherein said cell comprises a mammalian cell, a bird cell, a fish cell, a mollusks cell, or an amphibian cell.

176. The method of embodiment 175, wherein said mammalian cell comprises a porcine cell, a bovine cell, a bubaline cell, an ovine cell, a caprine cell, a cervine cell, a bisontine cell, a cameline cell, an elaphine cell, or a lapine cell.

177. The method of embodiment 176, wherein said mammalian cell comprises said porcine cell.

178. The method of any one of embodiments 92-177, wherein a molar ratio of polycation amino groups of said polyplex to nucleic acid phosphate groups of said polyplex is at least about 1.

179. The method of any one of embodiments 92-178, wherein a molar ratio of polycation amino groups of said polyplex to nucleic acid phosphate groups of said polyplex is at most about 60.

180. The method of any one of embodiments 92-179, wherein the composition comprises said nucleic acid, said polymeric material, and said lipid, wherein a first mass ratio of said polymeric material to said nucleic acid is no less than 1:1, and wherein a second mass ratio of said lipid to said polymeric material is no less than 1:1.

181. The method of embodiment 180, wherein said first mass ratio is no less than 2:1; and wherein said second mass ratio is no less than 2:1.

182. The method of embodiment 181, wherein said first mass ratio is no less than 100:1.

183. The method of any one of embodiments 92-182, wherein a ratio of said nucleic acid molecule and said cell in said contacting is at least about 0.001 ng per 10000 cells.

184. The method of embodiment 183, wherein a ratio of said nucleic acid molecule and said cell in said contacting is at least about 0.01 ng per 10000 cells.

185. The method of embodiment 184, wherein a ratio of said nucleic acid molecule and said cell in said contacting is at least about 0.1 ng per 10000 cells.

186. The method of any one of embodiments 92-185, wherein said change of protein expression within said cell comprises an increased expression of a transcript encoded by said nucleic acid molecule within said cell.

187. The method of embodiment 186, wherein said transcript encoded by said nucleic acid molecule within said cell is at least about 1%, 5%, 10%, 50%, 100%, 150%, 2-fold, 5-fold, 10-fold, 100-fold, or 1000-fold higher than (1) a level of said transcript within a cell that has not contacted with said saccharide; (2) a level of said transcript within a cell that has not contacted with said nucleic acid molecule; or (3) a combination thereof, as measured by quantitative real-time polymerase chain reaction (QRT-PCR).

188. The method of any one of embodiments 92-185, wherein said change of protein expression within said cell comprises an increased expression of a transcript not encoded by said nucleic acid molecule within said cell.

189. The method of embodiment 188, wherein said transcript not encoded by said nucleic acid molecule within said cell is at least about 1%, 5%, 10%, 50%, 100%, 150%, 2-fold, 5-fold, 10-fold, 100-fold, or 1000-fold higher than (1) a level of said transcript within a cell that has not contacted with said saccharide; (2) a level of said transcript within a cell that has not contacted with said nucleic acid molecule; or (3) a combination thereof, as measured by quantitative real-time polymerase chain reaction (QRT-PCR).

190. The method of any one of embodiments 92-185, wherein said change of protein expression within said cell comprises a decreased expression of a transcript not encoded by said nucleic acid molecule within said cell.

191. The method of embodiment 188, wherein said transcript not encoded by said nucleic acid molecule within said cell is at least about 1%, 5%, 10%, 20%, 30%, 40% 50%, 60%, 70%, 80%, 90%, or 99% lower than (1) a level of said transcript within a cell that has not contacted with said saccharide; (2) a level of said transcript within a cell that has not contacted with said nucleic acid molecule; or (3) a combination thereof, as measured by quantitative real-time polymerase chain reaction (QRT-PCR).

192. An edible product prepared by the method of any one of embodiments 92-191.

193. A pharmaceutical active ingredient prepared by the method of any one of embodiments 92-191.

194. A tissue prepared by the method of any one of embodiments 92-191.

While preferred embodiments of the present disclosure have been shown and described herein, it may be obvious to those skilled in the art that such embodiments are provided by way of example only. It is not intended that the disclosure be limited by the specific examples provided within the specification. While the disclosure has been described with reference to the aforementioned specification, the descriptions and illustrations of the embodiments herein are not meant to be construed in a limiting sense. Numerous variations, changes, and substitutions may now occur to those skilled in the art without departing from the disclosure. Furthermore, it shall be understood that all aspects of the disclosure are not limited to the specific depictions, configurations or relative proportions set forth herein which depend upon a variety of conditions and variables. It may be understood that various alternatives to the embodiments of the disclosure described herein may be employed in practicing the disclosure. It is therefore contemplated that the disclosure shall also cover any such alternatives, modifications, variations or equivalents. It is intended that the following claims define the scope of the disclosure and that methods and structures within the scope of these claims and their equivalents be covered thereby.

1.-194. (canceled)

195. A composition comprising a nucleic acid molecule and a modified saccharide,
wherein said nucleic acid molecule comprises a messenger ribonucleic acid (mRNA), a self-amplifying RNA (saRNA), an endless/circular RNA (eRNA), a trans-amplifying RNA (ta-RNA), a silencing ribonucleic acid (siRNA), a transfer ribonucleic acid (tRNA), a micro ribonucleic acid (miRNA), a deoxyribonucleic acid (DNA), or a combination thereof;
wherein said modified saccharide comprises an aliphatic aldehyde modified saccharide, an aromatic aldehyde modified saccharide, a quaternary ammonium modified saccharide, a polyamine derivatized saccharide, a saccharide modified saccharide, a saccharide with a lipid additive, a phenol modified saccharide, a derivative thereof, or a combination thereof; and
wherein said nucleic acid and said modified saccharide are configured to facilitate a change in protein expression within a cell.

196. The composition of claim 195, wherein said modified saccharide comprises a chitosan, a hyaluronic acid, a pullulan, a heparin, an alginate, a starch, a cellulose, a hemicellulose, a gellan gum, a carrageenan, a dextran, a xanthan gum, an amylopectin, a derivative thereof, or a combination thereof.

197. The composition of claim 195, wherein said modified saccharide comprises a modified chitosan.

198. The composition of claim 195, wherein:
a. said aliphatic aldehyde modified saccharide comprises a hexanal aldehyde modified saccharide, a heptanal aldehyde modified saccharide, an octanal aldehyde modified saccharide, a nonanal aldehyde modified saccharide, a decanal aldehyde modified saccharide, a derivative thereof, or a combination thereof;
b. said aromatic aldehyde modified saccharide comprises a benzaldehyde modified saccharide, a cinnamaldehyde modified saccharide, a derivative thereof, or a combination thereof;
c. said polyamine derivatized saccharide comprises a spermine modified saccharide, a spermidine modified saccharide, a putrescine modified saccharide, diethylethylamine modified saccharide, a dimethylethylamine modified saccharide, a quaternary ammonium modified saccharide, an arginine modified saccharide, a derivative thereof, or a combination thereof;
d. said saccharide with said lipid additive comprises a saccharide with a lecithin, a saccharide with a phosphatidylcholine, a derivative thereof, or a combination thereof;
e. said saccharide modified saccharide comprises a monosaccharide modified saccharide or a derivative thereof;
f. said saccharide modified saccharide comprises a polysaccharide modified saccharide or a derivative thereof; or
g. said phenol modified saccharide comprises a chlorogenic acid modified saccharide, a ferulic acid modified saccharide, a caffeic acid modified saccharide, a gallic acid modified saccharide, a derivative thereof, or a combination thereof.

199. The composition of claim 198, wherein (1) said aliphatic aldehyde modified saccharide comprises said decanal aldehyde modified saccharide; (2) said monosaccharide modified saccharide comprises a lactose modified saccharide, a mannose modified saccharide, a glucose modified saccharide, a galactose modified saccharide, a glucosamine modified saccharide, a sucrose modified saccharide, a xylose modified saccharide, a ribose modified saccharide, a fructose modified saccharide, a glyceraldehyde modified saccharide, a derivative thereof, or a combination thereof; or (3) said polysaccharide modified saccharide comprises an anionic polysaccharide modified saccharide, a maltose modified saccharide, a reducing polysaccharide modified saccharide, a derivative thereof, or a combination thereof.

200. The composition of claim 199, wherein (1) said anionic polysaccharide modified saccharide comprises an alginate modified saccharide, a carboxymethylated cellulose modified saccharide, a hyaluronic acid modified saccharide, a pectin modified saccharide, a pullulan modified saccharide, a starch modified saccharide, a xanthan gum modified saccharide, a derivative thereof, or a combination thereof; or (2) said reducing polysaccharide modified saccharide comprises a maltodextrin modified saccharide, a cellobiose modified saccharide, a cationic lipid additive, a derivative thereof, or a combination thereof.

201. The composition of claim 195, wherein said nucleic acid molecule encodes MYOD1, MYOG, MYF5, MYF6, PAX3, PAX7, PPARY, adiponectin, FATP1-6, FABP4, GLUT4, Leptin, AdipoR1-2, CD137, a fragment thereof, or a variant thereof.

202. The composition of claim 195, wherein said nucleic acid molecule is monocistronic.

203. The composition of claim 195, wherein said nucleic acid molecule is polycistronic.

204. The composition of claim 195, wherein nucleic acid molecule comprises said miRNA or said siRNA comprising a polynucleotide sequence that is configured to facilitate (1) a reduction of pluripotency of said cell or (2) a differentiation of said cell.

205. The composition of claim 204, wherein said polynucleotide sequence comprises a polynucleotide sequence that comprises POUF51 (OCT3/4), KLF4, SOX2, or a complementary sequence thereof.

206. The composition of claim 205, wherein composition comprises a first nucleic acid molecule and a second nucleic molecule, wherein said first nucleic acid molecule is said mRNA or said saRNA, and wherein said second nucleic acid is said miRNA or said siRNA.

207. The composition of claim 195, wherein:
a. said change in said protein expression within said cell facilitates differentiation of said cell;
b. (New) The composition further comprises a lipid, an anionic polymer, or a combination thereof;
c. said modified saccharide is cationic in an aqueous solution or in a neutral solution;
d. said modified saccharide comprises cationic moieties having a cationic charge of at least about 0.5 mequivalent of basic group per gram;
e. said modified saccharide comprises cationic moieties having a cationic charge of at most about 20 mequiv/g;
f. said modified saccharide comprises an average molecular mass of at least about 20 kilodaltons (kDa);
g. said modified saccharide comprises an average molecular mass of at most about 2000 kDa;
h. said modified saccharide comprises a polysaccharide;
i. said modified saccharide and said nucleic acid molecule are configured to self-assemble with each other;
j. said modified saccharide and said nucleic acid molecule are configured to form a polyplex;
k. said nucleic acid molecule comprises an RNA-regulatory element;
l. said modified saccharide stabilizes said nucleic acid molecule;
m. said modified saccharide inhibits or reduces degradation of said nucleic acid molecule;
n. said nucleic acid molecule comprises a chemical modification;
o. said nucleic acid molecule comprises an unlocked nucleic acid;
p. said nucleic acid molecule comprises at least two types of nucleic acids;
q. said cell comprises a mammalian cell, a bird cell, a fish cell, a mollusks cell, or an amphibian cell; and/or
r. said modified saccharide comprises a mixture with a cationic lipid.

208. The composition of claim 207, wherein:
a. said nucleic acid molecule is at or near a surface of said polyplex;
b. said nucleic acid molecule is encapsulated within said polyplex;
c. said polyplex is regularly shaped, irregularly shaped, or branched;
d. an apparent diameter of said polyplex is at least about 5 nanometer (nm), as measured by dynamic light scattering (DLS) or microscopy;
e. an apparent diameter of said polyplex is at most about 5000 nm, as measured by said DLS or said microscopy;
f. said polyplex further comprises a nanoparticle;
g. said RNA-regulatory element comprises a transcriptional regulatory element, a co-transcriptional regulatory element, a post-transcriptional regulatory element, a translational regulatory element, or any combination thereof;
h. said RNA-regulatory element comprises a 5′-cap, 5′-untranslated region (UTR), a 3′-UTR, a poly-A tail modification, or any combination thereof;
i. said modified saccharide inhibits or reduces nuclease degradation of said nucleic acid molecule;
j. said mammalian cell comprises a porcine cell, a bovine cell, a bubaline cell, an ovine cell, a caprine cell, a cervine cell, a bisontine cell, a cameline cell, an elaphine cell, or a lapine cell;
k. a molar ratio of polycation amino groups of said polyplex to nucleic acid phosphate groups of said polyplex is at least about 1; and/or
l. a molar ratio of polycation amino groups of said polyplex to nucleic acid phosphate groups of said polyplex is at most about 60.

209. A method for facilitating a change in protein expression within said cell, comprising: contacting said cell with a composition of claim 1 under conditions sufficient for said cell to uptake said composition, thereby facilitating said change in protein expression within said cell.

210. The method of claim 209, wherein said cell comprises a mammalian cell, a bird cell, a fish cell, a mollusks cell, or an amphibian cell.

211. The method of claim 209, wherein a ratio of said nucleic acid molecule and said cell in said contacting is at least about 0.001 ng per 10000 cells, at least about 0.01 ng per 10000 cells, or at least about 0.1 ng per 10000 cells.

212. The method of claim 209, wherein:
a. said change of protein expression within said cell comprises an increased expression of a transcript encoded by said nucleic acid molecule within said cell;
b. said change of protein expression within said cell comprises an increased expression of a transcript not encoded by said nucleic acid molecule within said cell; or
c. said change of protein expression within said cell comprises a decreased expression of a transcript not encoded by said nucleic acid molecule within said cell.

213. The method of claim 212, wherein:
a. said transcript encoded by said nucleic acid molecule within said cell is at least about 1%, 5%, 10%, 50%, 100%, 150%, 2-fold, 5-fold, 10-fold, 100-fold, or 1000-fold higher than (1) a level of said transcript within a cell that has not contacted with said saccharide; (2) a level of said transcript within a cell that has not contacted with said nucleic acid molecule; or (3) a combination thereof, as measured by quantitative real-time polymerase chain reaction (QRT-PCR);
b. said transcript not encoded by said nucleic acid molecule within said cell is at least about 1%, 5%, 10%, 50%, 100%, 150%, 2-fold, 5-fold, 10-fold, 100-fold, or 1000-fold higher than (1) a level of said transcript within a cell that has not contacted with said saccharide; (2) a level of said transcript within a cell that has not contacted with said nucleic acid molecule; or (3) a combination thereof, as measured by QRT-PCR; or
c. said transcript not encoded by said nucleic acid molecule within said cell is at least about 1%, 5%, 10%, 20%, 30%, 40% 50%, 60%, 70%, 80%, 90%, or 99% lower than (1) a level of said transcript within a cell that has not contacted with said saccharide; (2) a level of said transcript within a cell that has not contacted with said nucleic acid molecule; or (3) a combination thereof, as measured by QRT-PCR.

214. The method of claim 209, further comprising, subsequent to said contacting, generating a tissue, an edible product, or a pharmaceutical active ingredient using said cell.
